(19) United States
(12) Patent Application Publication
(10) Pub. No.: US 2022/0409732 A1
(43) Pub. Date: Dec. 29, 2022

(54) PEPTIDE-MHC II PROTEIN CONSTRUCTS AND USES THEREOF

(71) Applicant: Regeneron Pharmaceuticals, Inc., Tarrytown, NY (US)

(72) Inventors: Douglas MacDonald, New York, NY (US); David Buckler, Sleepy Hollow, NY (US)

(73) Assignee: Regeneron Pharmaceuticals, Inc., Tarrytown, NY (US)

(21) Appl. No.: 17/780,158

(22) PCT Filed: Dec. 2, 2020
(86) PCT No.: PCT/US2020/062801

§ 371 Date: May 26, 2022

Related U.S. Application Data
(60) Provisional application No. 62/942,344, filed on Dec. 2, 2019.

(51) Int. Cl. A61K 47/64; A61K 47/65; A61P 37/04

(52) U.S. Cl. CPC A61K 47/646; A61K 47/65; A61P 37/04

(57) ABSTRACT

Compositions comprising an MHC ligand peptide covalently attached to an MHC class II molecule are provided herein. In some compositions, the MHC ligand peptide is covalently attached to the MHC class II molecule by a peptide linker, wherein the MHC ligand peptide or the peptide linker comprises a first cysteine, wherein the MHC class II a chain or a portion thereof or the MHC class II β chain or a portion thereof comprises a second cysteine, and wherein the first cysteine and the second cysteine form a disulfide bond such that the MHC ligand peptide is bound in a peptide-binding groove formed by the MHC class II a chain or the portion thereof and the MHC class II β chain or the portion thereof. Also provided are nucleic acids encoding such compositions and methods for using such compositions to elicit an immune response in a subject.

### CROSS REFERENCE TO RELATED APPLICATIONS

This application claims the benefit of U.S. Application No. 62/942,344, filed Dec. 2, 2019, which is herein incorporated by reference in its entirety for all purposes.

### REFERENCE TO A SEQUENCE LISTING SUBMITTED AS A TEXT FILE VIA EFS WEB

The Sequence Listing written in file 696193SEQLIST.txt is 50.9 kilobytes, was created on Nov. 11, 2020, and is hereby incorporated by reference.

### BACKGROUND

Soluble peptide-MHC I protein constructs have been previously described. These constructs can be used for various applications, including immunizing rodents (e.g., VELOCIMMUNE® rodents) to generate anti-peptide-in-groove antibodies. However, there is a need for better soluble peptide-MHC II protein constructs.

### SUMMARY

Compositions comprising an MHC ligand peptide covalently attached to an MHC class II molecule, nucleic acids encoding such compositions, and methods of using such compositions to elicit an immune response in a subject are provided.

In one aspect, provided are compositions comprising an MHC ligand peptide covalently attached to an MHC class II molecule comprising an MHC class II α chain or a portion thereof and an MHC class II β chain or a portion thereof. In some such compositions, the MHC ligand peptide is covalently attached to the MHC class II molecule by a peptide linker. In some such compositions, the MHC ligand peptide or the peptide linker comprises a first cysteine and the MHC class II molecule comprises a second cysteine. In some such compositions, the first cysteine and the second cysteine form a disulfide bond such that the MHC ligand peptide is bound in a peptide-binding groove formed by the MHC class II α chain or the portion thereof and the MHC class II β chain or the portion thereof.

In some such compositions, the MHC class II α chain or the portion thereof comprises an α1 domain, and the MHC class II β chain or the portion thereof comprises a 31 domain. Optionally, the MHC class II α chain or the portion thereof comprises an MHC class II α chain extracellular domain, and the MHC class II β chain or the portion thereof comprises an MHC class II β chain extracellular domain. Optionally, the MHC class II α chain or the portion thereof comprises the α1 domain, an α2 domain, a transmembrane domain, and a cytoplasmic domain. Optionally, the MHC class II β chain or the portion thereof comprises the β1 domain, a β2 domain, a transmembrane domain, and a cytoplasmic domain.

In some such compositions, the composition is membrane-anchored. In some such compositions, the composition is soluble. Optionally, the MHC class II α chain or the portion thereof comprises the α1 domain and an α2 domain but does not comprise a transmembrane domain or a cytoplasmic domain. Optionally, the MHC class II β chain or the portion thereof comprises the β1 domain and a β2 domain but does not comprise a transmembrane domain or a cytoplasmic domain. Optionally, the MHC class II α chain or the portion thereof and the MHC class II β chain or the portion thereof are linked by a Jun-Fos zipper, electrostatic engineering, knobs-into-holes, an immunoglobulin scaffold, an immunoglobulin Fc region, or a linker. Optionally, the MHC class II α chain or the portion thereof and the MHC class II β chain or the portion thereof are linked by a Jun-Fos zipper comprising a Jun leucine zipper dimerization motif and a Fos leucine zipper dimerization motif, and the MHC class II α chain or the portion thereof is linked to the Jun leucine zipper dimerization motif and the MHC class II β chain or the portion thereof is linked to the Fos leucine zipper dimerization motif, or the MHC class II α chain or the portion thereof is linked to the Fos leucine zipper dimerization motif and the MHC class II β chain or the portion thereof is linked to the Jun leucine zipper dimerization motif Optionally, the C-terminal end of the MHC class II α chain or the portion thereof is linked to the Jun leucine zipper dimerization motif and the C-terminal end of the MHC class II β chain or the portion thereof is linked to the Fos leucine zipper dimerization motif Optionally, the C-terminal end of the MHC class II α chain or the portion thereof is linked to the Fos leucine zipper dimerization motif and the C-terminal end of the MHC class II β chain or the portion thereof is linked to the Jun leucine zipper dimerization motif. Optionally, the MHC class II α chain or the portion thereof is linked to the Jun leucine zipper dimerization motif by an MHC-Jun linker, and the MHC class II β chain or the portion thereof is linked to the Fos leucine zipper dimerization motif by an MHC-Fos linker. Optionally, the MHC class II α chain or the portion thereof is linked to the Fos leucine zipper dimerization motif by the MHC-Fos linker, and the MHC class II β chain or the portion thereof is linked to the Jun leucine zipper dimerization motif by the MHC-Jun linker. Optionally, the MHC-Jun linker and the MHC-Fos linker each comprise the sequence set forth in SEQ ID NO: 1.

In some such compositions, the MHC ligand peptide is about 10 to about 18 amino acids in length, is about 10 to about 15 amino acids in length, or is about 10 to about 12 amino acids in length. In some such compositions, the MHC ligand peptide is 10 to 18 amino acids in length, is 10 to 15 amino acids in length, or is 10 to 12 amino acids in length. In some such compositions, the MHC ligand peptide comprises residues P-1 to P9 or residues P-3 to P9. In some such compositions, the MHC ligand peptide is an antigenic MHC ligand peptide. In some such compositions, the MHC ligand peptide is associated with a T-cell-mediated disease.

In some such compositions, the peptide linker linking the MHC ligand peptide to the MHC class II molecule is a flexible linker. In some such compositions, the peptide linker linking the MHC ligand peptide to the MHC class II molecule comprises one or more flexible amino acids and one or more polar amino acids. In some such compositions, the peptide linker linking the MHC ligand peptide to the MHC class II molecule does not comprise any charged amino acids. In some such compositions, the peptide linker linking the MHC ligand peptide to the MHC class II molecule comprises a cleavage site. Optionally, the cleavage site is a tobacco etch virus (TEV) protease cleavage site.

In some such compositions, the peptide linker linking the MHC ligand peptide to the MHC class II molecule is non-immunogenic. In some such compositions, the peptide linker linking the MHC ligand peptide to the MHC class II molecule is connected to the N-terminal end of the MHC class II β chain or the portion thereof. In some such compositions, the peptide linker linking the MHC ligand peptide to the MHC class II molecule is connected to the N-terminal end of the MHC class II α chain or the portion thereof. In some such compositions, the peptide linker linking the MHC ligand peptide to the MHC class II molecule is at least about 9 amino acids in length. In some such compositions, the peptide linker linking the MHC ligand peptide to the MHC class II molecule is at least 9 amino acids in length. In some such compositions, the peptide linker linking the MHC ligand peptide to the MHC class II molecule is between about 9 and about 50 amino acids in length. In some such compositions, the peptide linker linking the MHC ligand peptide to the MHC class II molecule is between 9 and 50 amino acids in length. In some such compositions, the peptide linker linking the MHC ligand peptide to the MHC class II molecule comprises 2-4 repeats of the sequence set forth in SEQ ID NO: 4.

In some such compositions, the peptide linker linking the MHC ligand peptide to the MHC class II molecule comprises the first cysteine. Optionally, the first cysteine is the only cysteine in the peptide linker linking the MHC ligand peptide to the MHC class II molecule. Optionally, the first cysteine is in the first four amino acids of the peptide linker linking the MHC ligand peptide to the MHC class II molecule. In some such compositions, the peptide linker linking the MHC ligand peptide to the MHC class II molecule comprises 2-4 repeats of the sequence set forth in SEQ ID NO: 4, wherein one amino acid in one of the repeats is mutated to cysteine. Optionally, the peptide linker linking the MHC ligand peptide to the MHC class II molecule comprises the sequence set forth in SEQ ID NO: 21.

In some such compositions, the MHC ligand peptide comprises the first cysteine. Optionally, the first cysteine faces away from an epitope formed by the composition.

In some such compositions, the second cysteine is in the MHC class II α chain or the portion thereof. Optionally, the peptide linker linking the MHC ligand peptide to the MHC class II molecule is connected to the N-terminal end of the MHC class II β chain or the portion thereof.

In some such compositions, the second cysteine is not present in a wild type MHC class II molecule corresponding to the MHC class II molecule in the composition. Optionally, the second cysteine is in place of a non-cysteine amino acid in the corresponding wild type MHC class II molecule. Optionally, the second cysteine is in the MHC class II α chain or the portion thereof. Optionally, the second cysteine is at a position corresponding to position 101 in the sequence set forth in SEQ ID NO: 49 when the MHC class II α chain or the portion thereof is optimally aligned with SEQ ID NO: 49. For example, the second cysteine can be a position corresponding to the position labeled DQA1 R101 in the alignment of HLA-DPA1, HLA-DQA1, and HLA-DRA1 full-length sequences in FIG. 3. For example, the second cysteine in the corresponding wild type MHC class II α chain can be at a position corresponding to position 78 in the sequence set forth in SEQ ID NO: 59 when the MHC class II α chain or the portion thereof is optimally aligned with SEQ ID NO: 59.

In some such compositions, the MHC class II molecule lacks a cysteine present in a corresponding wild type MHC class II molecule. Optionally, the cysteine present in the corresponding wild type MHC class II molecule has been replaced with any other amino acid. Optionally, the cysteine present in the corresponding wild type MHC class II molecule has been replaced with alanine, glutamine, tryptophan, or arginine. Optionally, the cysteine present in the corresponding wild type MHC class II molecule has been replaced with an alanine or a glutamine in the MHC class II molecule in the composition.

In some such compositions, the MHC class II α chain or the portion thereof lacks a cysteine present in a corresponding wild type MHC class II α chain. Optionally, the cysteine present in the corresponding wild type MHC class II α chain has been replaced with an alanine or a glutamine in the MHC class II α chain or the portion thereof in the composition. Optionally, the cysteine in the corresponding wild type MHC class II α chain is at a position corresponding to position 70 in the sequence set forth in SEQ ID NO: 49 when the MHC class II α chain or the portion thereof is optimally aligned with SEQ ID NO: 49. For example, the cysteine in the corresponding wild type MHC class II α chain can be at a position corresponding to the position labeled DQA1 C70 in the alignment of HLA-DPA1, HLA-DQA1, and HLA-DRA1 full-length sequences in FIG. 3. For example, the cysteine in the corresponding wild type MHC class II α chain can be at a position corresponding to position 47 in the sequence set forth in SEQ ID NO: 59 when the MHC class II α chain or the portion thereof is optimally aligned with SEQ ID NO: 59.

In some such compositions, the composition further comprises one or more immunostimulatory molecules. Optionally, the one or more immunostimulatory molecules is a T cell epitope that induces a T cell mediated immune response to the composition. Optionally, the one or more immunostimulatory molecules comprise a pan-DR-binding epitope (PADRE) and/or a peptide from lymphocytic choriomeningitis virus (LCMV). Optionally, the one or more immunostimulatory molecules are directly or indirectly covalently linked to the MHC class II molecule. Optionally, the one or more immunostimulatory molecules are directly or indirectly covalently linked to the MHC class II α chain or the portion thereof and/or the MHC class II β chain or the portion thereof.

In some such compositions, the MHC class II molecule is a human MHC class II molecule. Optionally, the human MHC class II molecule is selected from the group consisting of HLA-DQ, HLA-DR, and HLA-DP. Optionally, the human MHC class II molecule is an HLA-DQ2 molecule. Optionally, the human MHC class II molecule is an HLA-DR2 molecule.

In some such compositions, the MHC class II α chain or the portion thereof comprises an MHC class II α chain extracellular domain, and the MHC class II β chain or the portion thereof comprises an MHC class II β chain extracellular domain, the peptide linker linking the MHC ligand peptide to the MHC class II molecule is a flexible linker between about 9 and about 50 amino acids in length that comprises the first cysteine and is connected to the N-terminal end of the MHC class II β chain or the portion thereof, the second cysteine is in the MHC class II α chain or the portion thereof and is not present in a wild type MHC class II molecule corresponding to the MHC class II molecule in the composition, and the MHC class II molecule lacks a cysteine present in a corresponding wild type MHC class II molecule. In some such compositions, the MHC class II α chain or the portion thereof comprises an MHC class II α chain extracellular domain, and the MHC class II β chain or the portion thereof comprises an MHC class II β chain extracellular domain, the peptide linker linking the MHC ligand peptide to the MHC class II molecule is a flexible linker between 9 and 50 amino acids in length that comprises the first cysteine and is connected to the N-terminal end of the MHC class II β chain or the portion thereof, the second cysteine is in the MHC class II α chain or the portion thereof and is not present in a wild type MHC class II molecule corresponding to the MHC class II molecule in the composition, and the MHC class II molecule lacks a cysteine present in a corresponding wild type MHC class II molecule. Optionally, the composition is soluble, the MHC class II α chain or the portion thereof comprises the α1 domain and an α2 domain but does not comprise a transmembrane domain or a cytoplasmic domain, the MHC class II β chain or the portion thereof comprises the β1 domain and a β2 domain but does not comprise a transmembrane domain or a cytoplasmic domain, and the MHC class II α chain or the portion thereof and the MHC class II β chain or the portion thereof are linked by a Jun-Fos zipper comprising a Jun leucine zipper dimerization motif and a Fos leucine zipper dimerization motif. Optionally, the second cysteine is at a position corresponding to position 101 in the sequence set forth in SEQ ID NO: 49 when the MHC class II α chain or the portion thereof is optimally aligned with SEQ ID NO: 49, and the cysteine in the corresponding wild type MHC class II molecule is at a position corresponding to position 70 in the sequence set forth in SEQ ID NO: 49 when the MHC class II α chain or the portion thereof is optimally aligned with SEQ ID NO: 49. For example, the second cysteine can be a position corresponding to the position labeled DQA1 R101 in the alignment of HLA-DPA1, HLA-DQA1, and HLA-DRA1 full-length sequences in FIG. 3, and the cysteine in the corresponding wild type MHC class II α chain can be at a position corresponding to the position labeled DQA1 C70 in the alignment of HLA-DPA1, HLA-DQA1, and HLA-DRA1 full-length sequences in FIG. 3. For example, the second cysteine in the corresponding wild type MHC class II α chain can be at a position corresponding to position 78 in the sequence set forth in SEQ ID NO: 59 when the MHC class II α chain or the portion thereof is optimally aligned with SEQ ID NO: 59, and the cysteine in the corresponding wild type MHC class II α chain can be at a position corresponding to position 47 in the sequence set forth in SEQ ID NO: 59 when the MHC class II α chain or the portion thereof is optimally aligned with SEQ ID NO: 59. Optionally, the MHC class II molecule is a human MHC class II molecule is selected from the group consisting of HLA-DQ, HLA-DP, and HLA-DR. Optionally, the human MHC class II molecule is HLA-DQ.

Optionally, the MHC class II α chain extracellular domain comprises SEQ ID NO: 64. Optionally, the MHC class II α chain extracellular domain consists essentially of SEQ ID NO: 64. Optionally, the MHC class II α chain extracellular domain consists of SEQ ID NO: 64. Optionally, the MHC class II α chain or the portion thereof (e.g., MHC class II α chain extracellular domain) is linked (e.g., on the C-terminal end) to a Fos leucine zipper dimerization motif Optionally, the Fos leucine zipper dimerization motif comprises SEQ ID NO: 23. Optionally, the Fos leucine zipper dimerization motif consists essentially of SEQ ID NO: 23. Optionally, the Fos leucine zipper dimerization motif consists of SEQ ID NO: 23. Optionally, the MHC class II β chain extracellular domain comprises SEQ ID NO: 60. Optionally, the MHC class II β chain extracellular domain consists essentially of SEQ ID NO: 60. Optionally, the MHC class II β chain extracellular domain consists of SEQ ID NO: 60. Optionally, the MHC class II β chain or the portion thereof (e.g., MHC class II β chain extracellular domain) is linked (e.g., on the C-terminal end) to a Jun leucine zipper dimerization motif. Optionally, the Jun leucine zipper dimerization motif comprises SEQ ID NO: 24. Optionally, the Jun leucine zipper dimerization motif consists essentially of SEQ ID NO: 24. Optionally, the Jun leucine zipper dimerization motif consists of SEQ ID NO: 24. Optionally, the MHC class II β chain extracellular domain is linked to the Jun leucine zipper dimerization motif by a linker. Optionally, the linker comprises SEQ ID NO: 1. Optionally, the linker consists essentially of SEQ ID NO: 1. Optionally, the linker consists of SEQ ID NO: 1. Optionally, the N-terminal end of the MHC class II β chain or the portion thereof (e.g., MHC class II β chain extracellular domain) is linked to the MHC ligand peptide (e.g., the C-terminal end of the MHC ligand peptide) by a linker. Optionally, the linker comprises SEQ ID NO: 21. Optionally, the linker consists essentially of SEQ ID NO: 21. Optionally, the linker consists of SEQ ID NO: 21. Optionally, the MHC ligand peptide is about 10 to about 18 amino acids in length, is about 10 to about 15 amino acids in length, or is about 10 to about 12 amino acids in length, and/or optionally the MHC ligand peptide comprises residues P-1 to P9 or residues P-3 to P9. Optionally, the MHC ligand peptide is 10 to 18 amino acids in length, is 10 to 15 amino acids in length, or is 10 to 12 amino acids in length, and/or optionally the MHC ligand peptide comprises residues P-1 to P9 or residues P-3 to P9.

In another aspect, provided are nucleic acids encoding any of the above compositions.

In another aspect, provided are methods of eliciting an immune response in a subject. Some such methods comprise administering to the subject an effective amount of any of the above compositions or a nucleic acid encoding the composition.

In another aspect, provided are methods of generating an antigen-binding protein. Some such methods comprise: (a) immunizing a non-human animal with any of the above compositions or a nucleic acid encoding the composition; and (b) maintaining the non-human animal in conditions sufficient for the non-human animal to mount an immune response to the composition. Optionally, the antigen-binding protein specifically binds an antigenic composition comprising an MHC ligand peptide covalently attached to an MHC class II molecule. In some embodiments, the antigen-binding protein is an immunoglobulin molecule or a fragment thereof. In some embodiments, the antigen-binding protein is a T cell receptor molecule or a fragment thereof. In another aspect, provided are methods of generating an antigen-binding protein that specifically binds an antigenic composition comprising an MHC ligand peptide covalently attached to an MHC class II molecule. Some such methods comprise: (a) immunizing a non-human animal with any of the above compositions or a nucleic acid encoding the composition; and (b) maintaining the non-human animal in conditions sufficient for the non-human animal to mount an immune response to the composition. In some embodiments, the antigen-binding protein is an immunoglobulin molecule or a fragment thereof. In some embodiments, the antigen-binding protein is a T cell receptor molecule or a fragment thereof.

### BRIEF DESCRIPTION OF THE FIGURES

FIG. 1 (not to scale) shows some embodiments of a variety of soluble peptide-MHC II constructs. The sequence of the linker used in some of the constructs (SGGGGG) to link the Fos and Jun leucine zipper dimerization motifs to the alpha and beta chains is set forth in SEQ ID NO: 1. Labels indicate cysteines engineered into the linker (Linker Cys) and the alpha chain (R101C) in Construct B for disulfide stapling of the peptide. Labels also indicate mutation of cysteine at position 70 in the alpha chain to glutamine (C70Q) or alanine (C70A). The asterisk indicates a Davis-body modification (a CH3 modification that allows for differential binding of Fc to Protein A).

FIG. 2 shows an alignment of the full-length DQ2 alpha chain segments comprising no mutations, a C70Q mutation, or R101C and C70A mutations.

FIG. 3 shows an alignment of full-length alpha chain segments from different HLA class II alleles.

FIG. 4 shows results from a Biacore assay showing that, in some embodiments, soluble construct C binds to anti-class II monoclonal antibodies captured on an anti-mFc sensor surface.

FIG. 5 shows results from a Biacore assay showing that, in some embodiments, soluble construct C captured on an anti-hFc sensor surface binds to anti-class II monoclonal antibodies.

FIGS. 6A and 6B show, in some embodiments, soluble constructs in which peptides are tethered to the HLA-DQB chain and the HLA alpha and beta chains are dimerized either in a Jun/Fos or Fc knob-into-hole arrangement.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” (amino-terminus) and a “C-terminus” (carboxy-terminus or carboxyl-terminus). The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

A “promoter” is a regulatory region of DNA usually comprising a TATA box capable of directing RNA polymerase II to initiate RNA synthesis at the appropriate transcription initiation site for a particular polynucleotide sequence.

In some embodiments of the present invention, a promoter may additionally comprise other regions which influence the transcription initiation rate. The promoter sequences disclosed in some embodiments herein modulate transcription of an operably linked polynucleotide. A promoter can be active in one or more of the cell types disclosed in some embodiments herein (e.g., but not limited to, a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., but not limited to, a developmentally regulated promoter), or a spatially restricted promoter (e.g., but not limited to, a cell-specific or tissue-specific promoter).

“Operable linkage” or being “operably linked” includes juxtaposition of two or more components (e.g., but not limited to, a promoter and another sequence element) such that both components function normally and allow the possibility that at least one of the components can mediate a function that is exerted upon at least one of the other components. As a non-limiting example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., but not limited to, a regulatory sequence can act at a distance to control transcription of the coding sequence).

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell.

In some embodiments of the present invention, the term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. As a non-limiting example, a nucleic acid encoding a protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. As a non-limiting example, an “HLA locus” may refer to the specific location of an HLA gene, HLA DNA sequence, HLA-encoding sequence, or HLA position on a chromosome of the genome of an organism that has been identified as to where such a sequence resides. An “HLA locus” may comprise a regulatory element of an HLA gene, including, as a non-limiting example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

The methods and compositions provided herein employ a variety of different components. Some components throughout the description can have active variants and fragments. Such components include, for example, MHC class II molecules. Biological activity for each of these components is described elsewhere herein. The term “functional” refers to the innate ability of a protein or nucleic acid (or a fragment or variant thereof) to exhibit a biological activity or function. Such biological activities or functions can include, for example, the ability of an MHC class II molecule to bind to an MHC ligand peptide and/or to bind to a T-cell receptor (TCR) and effect a T cell response. The biological functions of functional fragments or variants may be the same or may in fact be changed (e.g., but not limited to, specificity or selectivity or efficacy) in comparison to the original molecule, but with retention of the molecule's basic biological function.

The term “wild type” includes entities having a structure (e.g., but not limited to, nucleotide sequence or amino acid sequence sequence) as found in a normal (as contrasted with mutant, diseased, altered, or so forth) state or context. Wild type genes and polypeptides often exist in multiple different forms (e.g., alleles).

The term “variant” refers to a nucleotide sequence differing from the sequence most prevalent in a population (e.g., but not limited to, by one nucleotide) or a protein sequence different from the sequence most prevalent in a population (e.g., but not limited to, by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. Non-limiting examples of a protein fragment can include an N-terminal fragment (i.e., removal of a portion of the C-terminal end of the protein), a C-terminal fragment (i.e., removal of a portion of the N-terminal end of the protein), or an internal fragment (i.e., removal of a portion of an internal portion of the protein).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., but not limited to, charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, as a non-limiting example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “in vitro” includes artificial environments and processes or reactions that occur within an artificial environment (e.g., but not limited to, a test tube or an isolated cell or cell line). The term “in vivo” includes natural environments (e.g., but not limited to, an organism or body or a cell or tissue within an organism or body) and to processes or reactions that occur within a natural environment. The term “ex vivo” includes cells that have been removed from the body of an individual and processes or reactions that occur within such cells.

The terms “major histocompatibility complex” and “MHC” encompass the terms “human leukocyte antigen” or “HLA” (the latter two of which are generally reserved for human MHC molecules), naturally occurring MHC molecules, individual chains of MHC molecules (e.g., but not limited to, MHC class I a (heavy) chain, 32 microglobulin, MHC class II α chain, and MHC class II β chain), individual subunits of such chains of MHC molecules (e.g., but not limited to, α1, α2, and/or α3 subunits of MHC class I α chain, α1-α2 subunits of MHC class II α chain, 31-02 subunits of MHC class II β chain) as well as portions (e.g., but not limited to, the peptide-binding portions such as the peptide-binding grooves), mutants, and various derivatives thereof (including fusion proteins), wherein such portion, mutants, and derivatives retain the ability to display an antigenic peptide for recognition by a T-cell receptor (TCR) (e.g., but not limited to, an antigen-specific TCR). An MHC class I molecule comprises a peptide binding groove formed by the α1 and α2 domains of the heavy α chain that can stow a peptide of around 8-10 amino acids. Despite the fact that both classes of MHC bind a core of about 9 amino acids (e.g., but not limited to, 5 to 17 amino acids) within peptides, the open-ended nature of MHC class II peptide binding groove (the α1 domain of a class II MHC α polypeptide in association with the Rβ1 domain of a class II MHC β polypeptide) allows for a wider range of peptide lengths. Peptides binding MHC class II usually vary between 13 and 17 amino acids in length, though shorter or longer lengths are not uncommon. As a result, peptides may shift within the MHC class II peptide binding groove, changing which 9-mer sequence sits directly within the groove at any given time. Conventional identifications of particular MHC variants are used herein.

The term “antigen” refers to any agent (e.g., but not limited to, protein, peptide, polysaccharide, glycoprotein, glycolipid, nucleotide, portions thereof, or combinations thereof) that, when introduced into an immunocompetent host, is recognized by the immune system of the host and elicits an immune response by the host. A TCR recognizes a peptide presented in the context of an MHC as part of an immunological synapse. The peptide-MHC (pMHC) complex is recognized by the TCR, with the peptide (antigenic determinant) and the TCR idiotype providing the specificity of the interaction. Accordingly, the term “antigen” encompasses peptides presented in the context of MHCs (e.g., but not limited to, peptide-MHC complexes or pMHC complexes). The peptide displayed on MHC may also be referred to as an “epitope” or an “antigenic determinant.” The terms “peptide,” “antigenic determinant,” “epitopes,” and so forth encompass not only those presented naturally by antigen-presenting cells (APCs), but also any desired peptide so long as it is recognized by an immune cell (e.g., but not limited to, when presented appropriately to the cells of an immune system).

“Peptide-MHC class II complex,” “pMHC class II complex,” “peptide-in-groove,” and the like include (i) an MHC class II molecule (e.g., but not limited to, a human MHC class II molecule) or portion thereof (e.g., but not limited to, the peptide-binding groove thereof or the extracellular portion thereof), and (ii) an antigenic peptide, where the MHC class II molecule and the antigenic peptide are complexed in such a manner that the pMHC class II complex can specifically bind a T-cell receptor. A pMHC class II complex encompasses cell surface expressed pMHC class II complexes and soluble pMHC class II complexes. Upon administration to an animal of an antigenic pMHC class II complex (e.g., but not limited to, a complex comprising an MHC class II molecule complexed with a peptide, such as a peptide that is foreign to the animal into which the pMHC class II complex is administered), the animal is capable of generating an antibody response to the antigenic pMHC class II complex and/or generating a T cell response to the antigenic pMHC class II complex (i.e., generating T cell receptors specific for the pMHC class II complex). Such specific antigen-binding proteins may then be isolated and used as therapeutics to specifically modulate the specific T-cell receptor interaction with the antigenic pMHC class II complex. Although in some cases a soluble pMHC class II complex comprising a peptide (e.g., but not limited to, which is foreign to the host animal into which the pMHC class II complex is administered) complexed with an MHC class II molecule may not elicit a T-cell immune response due to the soluble nature of the administered pMHC class II complex, such a soluble pMHC class II complex may still be considered antigenic in that it may elicit a B-cell-mediated immune response that generates antigen-binding proteins that specifically bind the soluble pMHC class II complex.

As used herein, the term “effective amount” includes an amount effective, at dosages and for periods of time necessary, to achieve the desired result (e.g., but not limited to, sufficient to elicit or modulate an immune response). An effective amount of a peptide-MHC class II complex may vary according to factors such as disease state, age, and weight of the subject, and the ability of the peptide-MHC class II complex to elicit a desired response in the subject. Dosage regimens may be adjusted to provide the optimum response. An effective amount is also one in which any toxic or detrimental effects (e.g., but not limited to, side effects) of the peptide-MHC class II complex are outweighed by any therapeutically beneficial effects.

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values+5 of a stated value.

The term “and/or” refers to and encompasses any and all possible combinations of one or more of the associated listed items, as well as the lack of combinations when interpreted in the alternative (“or”).

The term “or” refers to any one member of a particular list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

Compositions comprising an MHC ligand peptide covalently attached to an MHC class II molecule are provided herein. In some embodiments of the present invention, the MHC class II molecule can comprise an MHC class II α chain or a portion or fragment or variant thereof and an MHC class II β chain or a portion or fragment or variant thereof. In some embodiments of the compositions, the MHC ligand peptide is covalently attached to the MHC class II molecule by a peptide linker. The MHC ligand peptide or the peptide linker can comprise a first cysteine, and the MHC class II α chain or the portion or fragment or variant thereof or the MHC class II β chain or the portion or fragment or variant thereof can comprise a second cysteine. In some embodiments, the first cysteine and the second cysteine can then form a disulfide bond such that the MHC ligand peptide is bound in a peptide-binding groove formed by the MHC class II α chain or the portion or fragment or variant thereof and the MHC class II β chain or the portion or fragment or variant thereof. Also provided are nucleic acids encoding such compositions and methods for using such compositions to elicit an immune response in a subject.

Soluble peptide-MHC I protein constructs have been previously described. These constructs can be used for various applications, including immunizing rodents (e.g., but not limited to, VELOCIMMUNE© rodents) to generate anti-peptide-in-groove antibodies, or generating T cell receptors specific for the peptide-MHC I protein. We have now designed a peptide-MHC II protein construct in which a and β chains of the MHC II molecule are anchored together and to a peptide in its groove. These can be used for various applications, for example, but not limited to, generation of soluble MHC II constructs to act as an immunogen as well as membrane-anchored MHC II proteins for other applications that include recruitment of T cells expressing MHC class II-peptide specific TCRs.

### II. Compositions Comprising Peptide-MHC Class II Complexes

In some embodiments of the present invention, various peptide-MHC class II complexes (pMHC complexes) are provided. Antigenic peptide-MHC class II complexes can be used, for example, to generate pMHC-specific antigen-binding proteins. Some such complexes comprise an MHC ligand peptide covalently attached to an MHC class II molecule comprising an MHC class II α chain or a portion or fragment or variant thereof and an MHC class II β chain or a portion or fragment or variant thereof. In some embodiments of the complexes, the MHC ligand peptide is covalently attached to the MHC class II molecule by a peptide linker. The MHC ligand peptide or the peptide linker can comprise a first cysteine, and the MHC class II molecule (e.g., but not limited to, MHC class II α chain or the portion or fragment or variant thereof or the MHC class II β chain or the portion or fragment or variant thereof) can comprise a second cysteine, and wherein the first cysteine and the second cysteine form a disulfide bond such that the MHC ligand peptide is bound in a peptide-binding groove formed by the MHC class II α chain or the portion or fragment or variant thereof and the MHC class II β chain or the portion or fragment or variant thereof.

In some embodiments, the MHC class II molecules useful as part of an antigenic peptide-MHC class II complex can include at least a portion or fragment or variant of an MHC class II α chain and at least a portion or fragment or variant of an MHC class II β chain (e.g., but not limited to, at least a portion or fragment or variant of the extracellular domain of the MHC class II α chain and at least a portion or fragment or variant of the extracellular domain of the MHC class II β chain), such that the portion or fragment or variant of the MHC class II α chain and the portion or fragment or variant of the MHC class II β chain form a peptide binding groove that can bind the MHC ligand peptide. As a non-limiting example, the MHC class II molecules useful as part of an antigenic peptide-MHC class II complex can include naturally occurring full-length MHCs as well as individual chains of MHCs (e.g., but not limited to, MHC class II α chain and MHC class II β chain), individual subunits of such chains of MHCs (e.g., but not limited to, α1-α2 subunits of MHC class II α chain and β1-β2 subunits of MHC class II β chain, or α1 subunit of MHC class II α chain and β1 subunit of MHC class II β chain) as well as fragments, mutants, and derivatives thereof (including fusion proteins), wherein such fragments, mutants, and derivatives retain the ability to display an antigenic determinant for recognition by an antigen-specific T-cell receptor (TCR). MHC class II molecules and the MHC class II molecules useful as part of an antigenic peptide-MHC class II complex are described in more detail elsewhere herein.

In some embodiments of the complexes, at least one chain of the MHC class II molecule (e.g., but not limited to, the MHC class II α chain or a portion or fragment or variant thereof or the MHC class II β chain or a portion or fragment or variant thereof) and the MHC ligand peptide are associated as a fusion protein. As a non-limiting example, the MHC class II molecule (e.g., but not limited to, the MHC class II β chain or the portion or fragment or variant thereof or the MHC class II α chain or the portion or fragment or variant thereof) and the MHC ligand peptide can be connected via a linker. Linkage of MHC class II molecules to MHC ligand peptides and suitable linkers for doing so are described in more detail below.

In some embodiments of the complexes, the MHC ligand peptide or a linker connecting the MHC ligand peptide to at least one chain of the MHC class II molecule (or a portion or fragment or variant thereof) are attached via a disulfide bridge. A disulfide bridge is a disulfide bond that extends between a pair of oxidized cysteines. Linkage of the MHC ligand peptide or a linker connecting the MHC ligand peptide to at least one chain of the MHC class II molecule (or a portion or fragment or variant thereof) via disulfide bridges is described in more detail below.

The peptide-MHC class II complexes disclosed in some embodiments herein can be membrane-bound or soluble. MHC class II molecules are naturally membrane-anchored heterodimers. The hydrophobic transmembrane regions of the α and β chains facilitate assembly of the heterodimer. Some of the peptide-MHC class II complexes herein are membrane-bound. As a non-limiting example, such membrane-bound peptide-MHC class II complexes can comprise MHC class II molecules that comprise transmembrane domains or comprise transmembrane and cytoplasmic domains. As a non-limiting example, the MHC class II molecules in the complex can comprise an α chain comprising a transmembrane domain or comprising a transmembrane domain and a cytoplasmic domain, and/or the MHC class II molecules can comprise a β chain comprising a transmembrane domain or comprising a transmembrane domain and a cytoplasmic domain.

In some embodiments, the peptide-MHC class II complexes can be soluble (i.e., not membrane-bound). As a non-limiting example, such soluble peptide-MHC class II complexes can comprise MHC class II molecules that do not comprise transmembrane domains or do not comprise transmembrane and cytoplasmic domains. As a non-limiting example, the MHC class II molecules in the complex can comprise an α chain that does not comprise a transmembrane domain or does not comprise a transmembrane domain and a cytoplasmic domain, and/or the MHC class II molecules can comprise a β chain that does not comprise a transmembrane domain or does not comprise a transmembrane domain and a cytoplasmic domain.

In some embodiments, soluble peptide-MHC class II complexes can further comprise other components to stabilize chain pairing between the MHC class II α chain or the portion or fragment or variant thereof and the MHC class II β chain or the portion or fragment or variant thereof. In some embodiments, non-limiting examples of mechanisms for stabilizing chain pairing include linkage to Jun-Fos zippers, linkage to immunoglobulin scaffolds, linkage to immunoglobulin Fc regions (e.g., but not limited to, immunoglobulin Fc hinge regions), immunoglobulin Fc knobs-into-holes mutations, electrostatic engineering such as immunoglobulin Fc charge mutations (including, but not limited to, charge reversal mutations), direct linkers (e.g., but not limited to, covalent linkage such as peptide linkers), or any combination thereof. Detailed descriptions and non-limiting examples of each of these mechanisms are provided elsewhere herein. However, any other suitable means for chain pairing can be used.

A. MHC Class II Molecules

In some embodiments of the present invention, any suitable MHC class II molecules can be used in the peptide-MHC class II complexes described herein. MHC molecules are generally classified into two categories: class I and class II MHC molecules. An MHC class II molecule or MHC class II protein is a heterodimeric integral membrane protein comprising one a chain and one p chain in noncovalent association. The α chain has two extracellular domains (α1 and α2) and two intracellular domains (a TM domain and a CYT domain). The β chain has two extracellular domains (β1 and β2) and two intracellular domains (a TM domain and CYT domain).

The domain organization of class II MHC molecules forms the antigenic determinant binding site (e.g., the peptide-binding portion or peptide-binding groove) of the MHC molecule. The peptide-binding groove refers to the portion of an MHC protein that forms a cavity in which a peptide (e.g., antigenic determinant) can bind. The conformation of a peptide-binding groove is capable of being altered upon binding of a peptide to enable proper alignment of amino acid residues important for TCR binding to the peptide-MHC (pMHC) complex.

In some embodiments of peptide-MHC class II complexes, the MHC class II molecules include portions or fragments or variants of class II MHC chains that are sufficient to form a peptide-binding groove. A peptide-binding groove of a class II protein can comprise portions or fragments or variants of the α1 and β1 domains capable of forming two β-pleated sheets and two α helices. A first portion of the α1 domain forms a first β-pleated sheet and a second portion of the α1 domain forms a first α helix. A first portion of the β1 domain forms a second β-pleated sheet and a second portion of the β1 domain forms a second α helix. X-ray crystallographic structure of class II protein with a peptide engaged in the binding groove of the protein shows that one or both ends of the engaged peptide can project beyond the MHC protein. See, e.g., Brown et al. (1993) Nature 364(6432):33-39, herein incorporated by reference in its entirety for all purposes. Thus, the ends of the α1 and β1 α helices of class II form an open cavity such that the ends of the peptide bound to the binding groove are not buried in the cavity.

Many human and mammalian MHCs are known. As non-limiting examples of some embodiments, a human MHC II α or β polypeptide may be derived from the α or β polypeptides of a functional human HLA molecule encoded by any of HLA-DP, HLA-DQ, HLA-DR, HLA-DM, or HLA-DO loci, or a combination thereof. A list of commonly used HLA antigens and alleles and a brief explanation of HLA nomenclature is described in Shankarkumar et al., “The Human Leukocyte Antigen (HLA) System,” Int. J. Hum. Genet. 4(2):91-103, (2004), herein incorporated by reference in its entirety for all purposes. Additional information regarding HLA nomenclature and various HLA alleles can be found in Holdsworth et al. (2009) Tissue Antigens 73(2):95-170 and Marsh (2019) Int. J. Immunogenet. 46(5):346-418, each of which is herein incorporated by reference in its entirety for all purposes.

In one exemplary embodiment, the MHC is human MHC class II molecule such as a cell-surface expressed human HLA molecule selected from the group consisting of HLA-DP, HLA-DR, HLA-DQ, and any combination thereof. As a non-limiting example, the peptide-MHC class II complex can comprise one or more MHC class II α chains or domains or portions or fragments or variants thereof (e.g., but not limited to, one or more human MHC class II α chains or domains or portions or fragments or variants thereof). As a non-limiting exemplary embodiment, the class II α chain can be HLA-DPA, HLA-DQA, or HLA-DRA. Likewise, in some embodiments, the peptide-MHC class II complex can comprise one or more MHC class II R chains or domains or portions or fragments or variants thereof (e.g., but not limited to, one or more human MHC class II β chains or domains or portions or fragments or variants thereof). As a non-limiting exemplary embodiment, the class II β chain can be HLA-DPB, HLA-DQB, or HLA-DRB.

In some embodiments, of particular interest are polymorphic human HLA alleles known to be associated with a number of human diseases (e.g., but not limited to, human autoimmune diseases). Specific polymorphisms in HLA loci have been identified that correlate with development of rheumatoid arthritis, type I diabetes, Hashimoto's thyroiditis, multiple sclerosis, myasthenia gravis, Graves' disease, systemic lupus erythematosus, celiac disease, Crohn's disease, ulcerative colitis, and other autoimmune disorders. See, e.g., de Bakker (2006) Nat. Genet. 38(10):1166-1172; Wong and Wen (2004) Diabetologia 47(9):1476-1487; Taneja and David (1998) J. Clin. Invest. 101(5):921-926; and International MHC and Autoimmunity Genetics Network (2009) Proc. Natl. Acad. Sci. U.S.A. 106(44):18680-18685, each of which is herein incorporated by reference in its entirety for all purposes. In some embodiments, a human MHC II polypeptide may be derived from a human HLA molecule known to be associated with a particular disease (e.g., but not limited to, an autoimmune disease).

In one exemplary embodiment, the human MHC class II molecule (e.g., but not limited to, the human MHC II α and β polypeptides or portions or fragments or variants thereof) is derived from human HLA-DR (e.g., but not limited to, HLA-DR2). Typically, HLA-DR α chains are monomorphic (e.g., the α chain of HLA-DR protein is encoded by HLA-DRA gene, such as the HLA-DRα*01 gene). On the other hand, the HLA-DR β chain is polymorphic. Thus, HLA-DR2 comprises an α chain encoded by the HLA-DRA gene and a β chain encoded by the HLADR1β*1501 gene. Any suitable HLA-DR sequences are encompassed herein, such as polymorphic variants exhibited in human population, sequences with one or more conservative or nonconservative amino acid modifications, and so forth.

In another exemplary embodiment, the human MHC class II molecule (e.g., but not limited to, the human MHC II α and β polypeptides or portions or fragments or variants thereof) is derived from human HLA-DQ (e.g., but not limited to, HLA-DQ2). HLA-DQ2 is a serotype group determined by the antibody recognition of the 32 subset of DQ β-chains. The β-chain of DQ is encoded by the HLA-DQB1 locus and DQ2 are encoded by the HLA-DQB1*02 allele group. This group contains two common alleles, DQB1*0201 and DQB1*0202. DQ2 β-chains combine with α-chains, encoded by genetically linked HLA-DQA1 alleles, to form the cis-haplotype isoforms. These isoforms, nicknamed DQ2.2 and DQ2.5, are also encoded by the DQA1*0201 and DQA1*0501 genes, respectively. DQ2.5 is one of the most predisposing factors for autoimmune disease. DQ2.5 is encoded, often, by a haplotype associated with a large number of diseases, e.g., autoimmune diseases. This haplotype, HLA A1-B8-DR3-DQ2, is associated with diseases in which HLA-DQ2 has suspect involvement. For example, DQ2 is directly involved in celiac disease.

In another embodiment, the human MHC class II molecule (e.g., but not limited to, the human MHC II α and β polypeptides or portions or fragments or variants thereof) may be encoded by nucleotide sequences, or portions or fragments thereof, of HLA alleles known to be associated with common human diseases. Such HLA alleles include, but are not limited to, HLA-DRB1*0401, HLA-DRB1*0301, HLA-DQA1*0501, HLA-DQB1*0201, HLA-DRB1*1501, HLA-DRB1*1502, HLA-DQB1*0602, HLA-DQA1*0102, HLA-DQA1*0201, HLA-DQB1*0202, HLA-DQA1*0501, and combinations thereof. A summary of HLA allele/disease associations is provided in de Bakker (2006) Nat. Genet. 38(10):1166-1172, herein incorporated by reference in its entirety for all purposes. Further non-limiting examples of HLA alleles associated with common diseases include B*0801/DRB1*0301/DQA1*0501/DQB1*0201 (Graves' disease or myasthenia gravis), DRB1*1501/DQB1*0602 (multiple sclerosis), DQA1*0102 (multiple sclerosis), C*0602 (psoriasis), DQA1*0201/DQB1*0202 (DQ2.2) (celiac disease), DQA1*0501/DQB1*0201 (DQ2.5) (celiac disease), DRB1*1501 (systemic lupus erythematosus (SLE)), DRB1*0301 (type 1 diabetes or SLE), and B*5701 (abacavir hypersensitivity).

In some embodiments, the MHC class II molecules useful as part of an antigenic peptide-MHC class II complex include an MHC class II α chain or a portion or fragment or variant thereof and an MHC class II β chain or a portion or fragment or variant thereof (e.g., but not limited to, the extracellular domain of the MHC class II α chain or a portion or fragment or variant thereof and the extracellular domain of the MHC class II β chain or a portion or fragment or variant thereof), such that said MHC class II α chain and β chains (or portions or fragments or variants thereof) form a peptide-binding groove that can bind the MHC ligand peptide. As a non-limiting example, the MHC class II molecules useful as part of an antigenic peptide-MHC class II complex include naturally occurring full-length MHCs as well as individual chains of MHCs (e.g., but not limited to, MHC class II α chain and MHC class II β chain), individual subunits of such chains of MHCs (e.g., but not limited to, α1-α2 subunits of MHC class II α chain and β1-β2 subunits of MHC class II β chain, or α1 subunit of MHC class II α chain and β1 subunit of MHC class II β chain) as well as portions, fragments, mutants, and various derivatives thereof (including fusion proteins), wherein such portions, fragments, mutants, and derivatives retain the ability to display an antigenic determinant for recognition by an antigen-specific TCR. In one exemplary embodiment, the MHC class II α chain or portion or fragment or variant thereof and the MHC class II β chain or portion or fragment or variant thereof included in the peptide-MHC class II complex comprise the regions needed or the minimal regions needed to form a peptide-binding groove for the MHC ligand peptide. In one exemplary embodiment, the MHC class II α chain or portion or fragment or variant thereof and the MHC class II β chain or portion or fragment or variant thereof included in the peptide-MHC class II complex consist essentially of the regions needed or the minimal regions needed to form a peptide-binding groove for the MHC ligand peptide. In one exemplary embodiment, the MHC class II α chain or portion or fragment or variant thereof and the MHC class II β chain or portion or fragment or variant thereof included in the peptide-MHC class II complex consist of the regions needed or the minimal regions needed to form a peptide-binding groove for the MHC ligand peptide.

The MHC class II molecules in the peptide-MHC complexes disclosed in some embodiments herein can be membrane-bound or soluble. MHC class II molecules are naturally membrane-anchored heterodimers. The hydrophobic transmembrane regions of the α and β chains facilitate assembly of the heterodimer. Some of the MHC class II molecules in the peptide-MHC class II complexes disclosed in some embodiments herein are membrane-bound. As a non-limiting example, such membrane-bound peptide-MHC class II complexes can comprise MHC class II molecules that comprise transmembrane domains or a portion or fragment or variant thereof or comprise transmembrane and cytoplasmic domains or portions or fragment or variants thereof. As a non-limiting example, the MHC class II molecules in the complex can comprise an α chain comprising a transmembrane domain or a portion or fragment or variant thereof or comprising a transmembrane domain and a cytoplasmic domain or portions or fragments or variants thereof, and/or the MHC class II molecules can comprise a β chain comprising a transmembrane domain or a portion or fragment or variant thereof or comprising a transmembrane domain and a cytoplasmic domain or portions or fragments or variants thereof.

In some embodiments, the MHC class II molecules in the peptide-MHC class II complexes disclosed in some embodiments herein can be soluble (i.e., not membrane-bound). In an exemplary embodiment, such soluble peptide-MHC class II complexes can comprise MHC class II molecules that do not comprise transmembrane domains or do not comprise transmembrane and cytoplasmic domains. In another exemplary embodiment, the MHC class II molecules in the complex can comprise an α chain or a portion or fragment or variant thereof that does not comprise a transmembrane domain or does not a transmembrane domain and a cytoplasmic domain, and/or the MHC class II molecules can comprise a β chain or a portion or fragment or variant thereof that does not comprise a transmembrane domain or does not comprise a transmembrane domain and a cytoplasmic domain.

In one embodiment, the α chain or a portion or fragment or variant thereof comprises a fragment of a full-length α chain that does not include the transmembrane domain or regions C-terminal to the transmembrane domain on the C-terminal end. In another embodiment, the α chain or a portion or fragment or variant thereof comprises a fragment of a full-length α chain that does not include the signal peptide on the N-terminal end and does not include the transmembrane domain or regions C-terminal to the transmembrane domain on the C-terminal end. In a non-limiting example, the α chain or a portion or fragment or variant thereof can be operably linked to a different signal peptide. In an exemplary embodiment, the α chain or a portion or fragment or variant thereof comprises amino acid residues 24-216 of SEQ ID NO: 49, SEQ ID NO: 53, SEQ ID NO: 54, SEQ ID NO: 55, SEQ ID NO: 56, or SEQ ID NO: 57 or a fragment of a full-length α chain corresponding to amino acids residues 24-216 of SEQ ID NO: 49, SEQ ID NO: 53, SEQ ID NO: 54, SEQ ID NO: 55, SEQ ID NO: 56, or SEQ ID NO: 57 (e.g., when the full-length α chain from which the α chain or a portion or fragment or variant thereof was derived is optimally aligned with SEQ ID NO: 49, SEQ ID NO: 53, or SEQ ID NO: 54, SEQ ID NO: 55, SEQ ID NO: 56, or SEQ ID NO: 57). In another embodiment, the α chain or a portion or fragment or variant thereof comprises amino acid residues 29-222 of SEQ ID NO: 51 or a fragment of a full-length α chain corresponding to amino acids residues 29-222 of SEQ ID NO: 51 (e.g., when the full-length α chain from which the α chain or a portion or fragment or variant thereof was derived is optimally aligned with SEQ ID NO: 51). In another embodiment, the α chain or a portion or fragment or variant thereof comprises amino acid residues 26-216 of SEQ ID NO: 52 or 58 or a fragment of a full-length α chain corresponding to amino acids residues 26-216 of SEQ ID NO: 52 or 58 (e.g., when the full-length α chain from which the α chain or a portion or fragment or variant thereof was derived is optimally aligned with SEQ ID NO: 52 or 58). In another embodiment, the α chain or a portion or fragment or variant thereof comprises the sequence set forth in any one of SEQ ID NOS: 59 and 61-68.

In one embodiment, the β chain or a portion or fragment or variant thereof comprises a fragment of a full-length β chain that does not include the transmembrane domain or regions C-terminal to the transmembrane domain on the C-terminal end. In another embodiment, the R chain or a portion or fragment or variant thereof comprises a fragment of a full-length β chain that does not include the signal peptide on the N-terminal end and does not include the transmembrane domain or regions C-terminal to the transmembrane domain on the C-terminal end. In a non-limiting example, the β chain or a portion or fragment or variant thereof can be operably linked to a different signal peptide. In an exemplary embodiment, the β chain or a portion or fragment or variant thereof comprises amino acid residues 33-230 of SEQ ID NO: 50 or a fragment of a full-length β chain corresponding to amino acids residues 33-230 of SEQ ID NO: 50 (e.g., when the full-length β chain from which the β chain or a portion or fragment or variant thereof was derived is optimally aligned with SEQ ID NO: 50). In another exemplary embodiment, the β chain or a portion or fragment or variant thereof comprises the sequence set forth in SEQ ID NO: 60.

In some embodiments, MHC II molecules in soluble peptide-MHC class II complexes can further comprise other components to stabilize chain pairing between the MHC class II α chain or portion or fragment or variant thereof and the MHC class II β chain or portion or fragment or variant thereof. In some embodiments, non-limiting examples of mechanisms for stabilizing chain pairing include linkage to Jun-Fos zippers, linkage to immunoglobulin scaffolds, linkage to immunoglobulin Fc regions (e.g., but not limited to, immunoglobulin Fc hinge regions), immunoglobulin Fc knobs-into-holes, electrostatic engineering such as immunoglobulin Fc charge mutations (including, but not limited to, charge reversal mutations), direct linkers (e.g., but not limited to, covalent linkage such as peptide linkers), or any combination thereof. However, any other suitable means for chain pairing can be used.

In one embodiment, the MHC class II α chain or portion or fragment or variant thereof and the MHC class II β chain or portion or fragment or variant thereof are linked by a Jun-Fos zipper. Synthetic peptides of the Fos and Jun leucine zipper dimerization motif are known to assemble as stable, soluble heterodimers. See, e.g., Kalandadze et al. (1996) J. Biol. Chem. 271:20156-20162 and Gauthier et al. (1998) Proc. Natl. Acad. Sci. U.S.A. 95:11828-11833, each of which is herein incorporated by reference in its entirety for all purposes. The leucine zippers are characterized by five leucines that are spaced periodically at every seventh residue (heptad repeat). Each heptad repeat contributes two turns of the alpha-helix. The leucine residues have a special function in leucine zipper dimerization and form the interface between the two alpha-helices of the coiled coil. The Jun/Fos heterodimer is soluble due to charged residues on the outer surface of the coiled coil. In one exemplary embodiment, the MHC class II α chain or portion or fragment or variant thereof and the MHC class II β chain or portion or fragment or variant thereof (e.g., but not limited to, the C-terminal end of the MHC class II α chain or portion or fragment or variant thereof and the C-terminal end of the MHC class II β chain or portion or fragment or variant thereof) can be linked to leucine zipper dimerization motifs from the transcription factors Fos and Jun, which assemble as a soluble, tightly packed coiled coil structure. In another exemplary embodiment, the hydrophobic transmembrane regions of the MHC class II α chain and MHC class II β chain are replaced by leucine zipper dimerization motifs from the transcription factors Fos and Jun. In yet another exemplary embodiment, the extracellular domain of the MHC class II α chain or a portion or fragment or variant thereof (e.g., but not limited to, comprising the MHC class II α1 domain or a portion or fragment or variant thereof or the MHC class II α1 and α2 domains or a portion or fragment or variant thereof) can be linked (e.g., but not limited to, fused in frame or via a linker) to the Fos leucine zipper dimerization motif, and the extracellular domain of the MHC class II β chain or a portion or fragment or variant thereof (e.g., but not limited to, comprising the MHC class II (β1 domain or a portion or fragment or variant thereof or the MHC class II 31 and 32 domains or a portion or fragment or variant thereof) can be linked (e.g., but not limited to, fused in frame or via a linker) to the Jun leucine zipper dimerization motif. In another exemplary embodiment, the extracellular domain of the MHC class II α chain or a portion or fragment or variant thereof (e.g., but not limited to, comprising the MHC class II α1 domain or a portion or fragment or variant thereof or the MHC class II α1 and α2 domains or a portion or fragment or variant thereof) can be linked (e.g., but not limited to, fused in frame or via a linker) to the Jun leucine zipper dimerization motif, and the extracellular domain of the MHC class II β chain or a portion or fragment or variant thereof (e.g., but not limited to, comprising the MHC class II β1 domain or a portion or fragment or variant thereof or the MHC class II β1 and β2 domains or a portion or fragment or variant thereof) can be linked (e.g., but not limited to, fused in frame or via a linker) to the Fos leucine zipper dimerization motif. The linkage (e.g., but not limited to, fusion in frame or via a linker) can be, for example, at the C-terminal end of the extracellular domain of the MHC class II α chain or a portion or fragment or variant thereof and the C-terminal end of the extracellular domain of the MHC class II β chain or a portion or fragment or variant thereof. Suitable linkers for linking the Jun leucine zipper dimerization motif and/or the Fos leucine zipper dimerization motif to the MHC class II molecule are disclosed in more detail elsewhere herein. Optionally, in a non-limiting example, the linker comprises SGGGGG (SEQ ID NO: 1). Optionally, in a non-limiting example, the linker consists essentially of SGGGGG (SEQ ID NO: 1). Optionally, in a non-limiting example, the linker consists of SGGGGG (SEQ ID NO: 1).

In some embodiments, exemplary sequences for the Fos leucine zipper dimerization motif and the Jun leucine zipper dimerization motif are set forth in SEQ ID NOS: 23 and 24, respectively.

As a non-limiting example, a Fos leucine zipper dimerization motif used in the compositions disclosed in some embodiments herein can comprise a sequence at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 23. As a non-limiting example, a Fos leucine zipper dimerization motif used in the compositions disclosed in some embodiments herein can consist essentially of a sequence at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 23. As a non-limiting example, a Fos leucine zipper dimerization motif used in the compositions disclosed in some embodiments herein can consist of a sequence at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 23. As a non-limiting example, a Fos leucine zipper dimerization motif used in the compositions disclosed in some embodiments herein can comprise a sequence at least 90%, at least 91%, at least 92%, at least 93%, at least 94%, at least 95%, at least 96%, at least 97%, at least 98%, at least 99%, or 100% identical to the sequence set forth in SEQ ID NO: 23. As a non-limiting example, a Fos leucine zipper dimerization motif used in the compositions disclosed in some embodiments herein can consist essentially of a sequence at least 90%, at least 91%, at least 92%, at least 93%, at least 94%, at least 95%, at least 96%, at least 97%, at least 98%, at least 99%, or 100% identical to the sequence set forth in SEQ ID NO: 23. As a non-limiting example, a Fos leucine zipper dimerization motif used in the compositions disclosed in some embodiments herein can consist of a sequence at least 90%, at least 91%, at least 92%, at least 93%, at least 94%, at least 95%, at least 96%, at least 97%, at least 98%, at least 99%, or 100% identical to the sequence set forth in SEQ ID NO: 23.

Likewise, in some embodiments, a Jun leucine zipper dimerization motif used in the compositions disclosed in some embodiments herein can comprise a sequence at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 24. Likewise, in some embodiments, a Jun leucine zipper dimerization motif used in the compositions disclosed in some embodiments herein can consist essentially of a sequence at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 24. Likewise, in some embodiments, a Jun leucine zipper dimerization motif used in the compositions disclosed in some embodiments herein can consist of a sequence at least about 90%, at least about 91%, at least about 92%, at least about 93%, at least about 94%, at least about 95%, at least about 96%, at least about 97%, at least about 98%, at least about 99%, or about 100% identical to the sequence set forth in SEQ ID NO: 24. Likewise, in some embodiments, a Jun leucine zipper dimerization motif used in the compositions disclosed in some embodiments herein can comprise a sequence at least 90%, at least 91%, at least 92%, at least 93%, at least 94%, at least 95%, at least 96%, at least 97%, at least 98%, at least 99%, or 100% identical to the sequence set forth in SEQ ID NO: 24. Likewise, in some embodiments, a Jun leucine zipper dimerization motif used in the compositions disclosed in some embodiments herein can consist essentially of a sequence at least 90%, at least 91%, at least 92%, at least 93%, at least 94%, at least 95%, at least 96%, at least 97%, at least 98%, at least 99%, or 100% identical to the sequence set forth in SEQ ID NO: 24. Likewise, in some embodiments, a Jun leucine zipper dimerization motif used in the compositions disclosed in some embodiments herein can consist of a sequence at least 90%, at least 91%, at least 92%, at least 93%, at least 94%, at least 95%, at least 96%, at least 97%, at least 98%, at least 99%, or 100% identical to the sequence set forth in SEQ ID NO: 24.

In some embodiments, a Fos leucine zipper dimerization motif used in the compositions disclosed herein is between 90% to 100% identical to the sequence set forth in SEQ ID NO: 23. In some embodiments, a Fos leucine zipper dimerization motif used in the compositions disclosed herein is between 92% to 100% identical to the sequence set forth in SEQ ID NO: 23. In some embodiments, a Fos leucine zipper dimerization motif used in the compositions disclosed herein is between 94% to 100% identical to the sequence set forth in SEQ ID NO: 23. In some embodiments, a Fos leucine zipper dimerization motif used in the compositions disclosed herein is between 96% to 100% identical to the sequence set forth in SEQ ID NO: 23. In some embodiments, a Fos leucine zipper dimerization motif used in the compositions disclosed herein is between 98% to 100% identical to the sequence set forth in SEQ ID NO: 23. In some embodiments, a Fos leucine zipper dimerization motif used in the compositions disclosed herein is between 90% to 98% identical to the sequence set forth in SEQ ID NO: 23. In some embodiments, a Fos leucine zipper dimerization motif used in the compositions disclosed herein is between 90% to 96% identical to the sequence set forth in SEQ ID NO: 23. In some embodiments, a Fos leucine zipper dimerization motif used in the compositions disclosed herein is between 90% to 94% identical to the sequence set forth in SEQ ID NO: 23. In some embodiments, a Fos leucine zipper dimerization motif used in the compositions disclosed herein is between 90% to 92% identical to the sequence set forth in SEQ ID NO: 23. In some embodiments, a Fos leucine zipper dimerization motif used in the compositions disclosed herein is between 92% to 98% identical to the sequence set forth in SEQ ID NO: 23. In some embodiments, a Fos leucine zipper dimerization motif used in the compositions disclosed herein is between 94% to 96% identical to the sequence set forth in SEQ ID NO: 23.

In some embodiments, a Jun leucine zipper dimerization motif used in the compositions disclosed herein is between 90% to 100% identical to the sequence set forth in SEQ ID NO: 24. In some embodiments, a Jun leucine zipper dimerization motif used in the compositions disclosed herein is between 92% to 100% identical to the sequence set forth in SEQ ID NO: 24. In some embodiments, a Jun leucine zipper dimerization motif used in the compositions disclosed herein is between 94% to 100% identical to the sequence set forth in SEQ ID NO: 24. In some embodiments, a Jun leucine zipper dimerization motif used in the compositions disclosed herein is between 96% to 100% identical to the sequence set forth in SEQ ID NO: 24. In some embodiments, a Jun leucine zipper dimerization motif used in the compositions disclosed herein is between 98% to 100% identical to the sequence set forth in SEQ ID NO: 24. In some embodiments, a Jun leucine zipper dimerization motif used in the compositions disclosed herein is between 90% to 98% identical to the sequence set forth in SEQ ID NO: 24. In some embodiments, a Jun leucine zipper dimerization motif used in the compositions disclosed herein is between 90% to 96% identical to the sequence set forth in SEQ ID NO: 24. In some embodiments, a Jun leucine zipper dimerization motif used in the compositions disclosed herein is between 90% to 94% identical to the sequence set forth in SEQ ID NO: 24. In some embodiments, a Jun leucine zipper dimerization motif used in the compositions disclosed herein is between 90% to 92% identical to the sequence set forth in SEQ ID NO: 24. In some embodiments, a Jun leucine zipper dimerization motif used in the compositions disclosed herein is between 92% to 98% identical to the sequence set forth in SEQ ID NO: 24. In some embodiments, a Jun leucine zipper dimerization motif used in the compositions disclosed herein is between 94% to 96% identical to the sequence set forth in SEQ ID NO: 24.

In another exemplary embodiment, the MHC class II α chain or a portion or fragment or variant thereof and the MHC class II β chain or portion or fragment or variant thereof are linked using an immunoglobulin scaffold (e.g., but not limited to, IgG scaffold). In one exemplary embodiment, the MHC class II α chain or portion or fragment or variant thereof and the MHC class II β chain or portion or fragment or variant thereof are linked to an immunoglobulin light chain variable region and an immunoglobulin heavy chain variable region, respectively, or vice versa. See, e.g., Hamad et al. (1998) J. Exp. Med. 188(9):1633-1640, herein incorporated by reference in its entirety for all purposes. In another exemplary embodiment, the hydrophobic transmembrane region of the MHC class II α chain and the hydrophobic transmembrane region of the MHC class II β chain are replaced by an immunoglobulin light chain variable region and an immunoglobulin heavy chain variable region, respectively, or vice versa. In another exemplary embodiment, the extracellular domain of the MHC class II α chain or a portion or fragment or variant thereof (e.g., but not limited to, comprising the MHC class II α1 domain or a portion or fragment or variant thereof or the MHC class II α1 and α2 domains or a portion or fragment or variant thereof) can be linked (e.g., but not limited to, fused in frame or via a linker) to an immunoglobulin light chain variable region, and the extracellular domain of the MHC class II β chain or a portion or fragment or variant thereof (e.g., but not limited to, comprising the MHC class II β1 domain or a portion or fragment or variant thereof or the MHC class II β1 and β2 domains or a portion or fragment or variant thereof) can be linked (e.g., but not limited to, fused in frame or via a linker) to an immunoglobulin heavy chain variable region. In another exemplary embodiment, the extracellular domain of the MHC class II α chain or a portion or fragment or variant thereof (e.g., but not limited to, comprising the MHC class II α1 domain or a portion or fragment or variant thereof or the MHC class II α1 and α2 domains or a portion or fragment or variant thereof) can be linked (e.g., but not limited to, fused in frame or via a linker) to an immunoglobulin heavy chain variable region, and the extracellular domain of the MHC class II β chain or a portion or fragment or variant thereof (e.g., but not limited to, comprising the MHC class II β1 domain or a portion or fragment or variant thereof or the MHC class II β1 and β2 domains or a portion or fragment or variant thereof) can be linked (e.g., but not limited to, fused in frame or via a linker) to an immunoglobulin light chain variable region. The linkage (e.g., but not limited to, fusion in frame or via a linker) can be, for example, at the C-terminal end of the extracellular domain of the MHC class II α chain or a portion or fragment or variant thereof and the C-terminal end of the extracellular domain of the MHC class II β chain or a portion or fragment or variant thereof. Suitable linkers are disclosed in more detail elsewhere herein.

In some embodiments, the MHC class II α chain or portion or fragment or variant thereof and/or the MHC class II β chain or portion or fragment or variant thereof can be linked to an immunoglobulin fragment crystallizable (Fc) region or fragment (e.g., but not limited to, an IgG2a Fc domain, such as a murine IgG2a Fc domain). See, e.g., Arnold et al. (2002) J. Immunol. Methods 271(1-2):137-151 and Appel et al. (2000) J. Biol. Chem. 275(1):312-321, each of which is herein incorporated by reference in its entirety for all purposes. In some embodiments, the Fc region or fragment can comprise a Davis-body modification (e.g., a CH3 modification that allows for differential binding of Fc to Protein A) for ease of purification. See, e.g., U.S. Pat. No. 8,586,713, incorporated herein by reference in its entirety for all purposes. As a non-limiting example, the Fc segment used can include the hinge, C_{H}2, and C_{H}3 domains (e.g., but not limited to, with the MHC class II α chain or portion or fragment or variant thereof or the MHC class II β chain or portion or fragment or variant thereof replacing the F(ab) arms of the antibody). The hinge region, for example, can increase mobility of the MHC class II α chain or portion or fragment or variant thereof and/or the MHC class II β chain or portion or fragment or variant thereof relative to the Fc segment comprising the C_{H}2 and C_{H}3 domains. In an exemplary embodiment, the hydrophobic transmembrane regions of the MHC class II α chain and/or the MHC class II β chain are replaced by an immunoglobulin Fc region or fragment. In one exemplary embodiment, the extracellular domain of the MHC class II α chain or a portion or fragment or variant thereof (e.g., but not limited to, comprising the MHC class II α1 domain or a portion or fragment or variant thereof or the MHC class II α1 and α2 domains or a portion or fragment or variant thereof) can be linked (e.g., but not limited to, fused in frame or via a linker) to an immunoglobulin Fc region or fragment, and/or the extracellular domain of the MHC class II β chain or a portion or fragment or variant thereof (e.g., but not limited to, comprising the MHC class II β1 domain or a portion or fragment or variant thereof or the MHC class II β1 and β2 domains or a portion or fragment or variant thereof) can be linked (e.g., but not limited to, fused in frame or via a linker) to an immunoglobulin Fc region or fragment. The linkage (e.g., but not limited to, fusion in frame or via a linker) can be, for example, at the C-terminal end of the extracellular domain of the MHC class II α chain or a portion or fragment or variant thereof and the C-terminal end of the extracellular domain of the MHC class II β chain or a portion or fragment or variant thereof. Suitable linkers are disclosed in more detail elsewhere herein.

Optionally, in some embodiments, the MHC class II α chain or portion or fragment or variant thereof and/or the MHC class II β chain or portion or fragment or variant thereof can further be linked to an immunoglobulin fragment crystallizable (Fc) region or portion or fragment or variant thereof (e.g., but not limited to, allowing for production of bivalent molecules). See, e.g., Arnold et al. (2002) J. Immunol. Methods 271(1-2):137-151 and Appel et al. (2000) J. Biol. Chem. 275(1):312-321, each of which is herein incorporated by reference in its entirety for all purposes. As a non-limiting example, the Fc segment used can include the hinge, C_{H}2, and C_{H}3 domains. In one exemplary embodiment, the immunoglobulin Fc region or fragment can be linked (e.g., but not limited to, fused or via a linker) to the C-terminal end of the Fos leucine zipper dimerization motif and/or can be linked to the C-terminal end of the Jun leucine zipper dimerization motif. Suitable linkers are disclosed elsewhere herein.

In another exemplary embodiment, the MHC class II α chain or portion or fragment or variant thereof and the MHC class II β chain or portion or fragment or variant thereof are linked using a knobs-into-holes strategy. Knobs-into-holes is a strategy for heterodimerization in which knob and hole variants are designed to heterodimerize by virtue of the knob inserting into an appropriately designed hole on the partner chain or domain. See, e.g., Ridgway et al. (1996) Protein Engineering 9(7):617-621, herein incorporated by reference in its entirety for all purposes. As a non-limiting example, knobs and holes can be designed in immunoglobulin Fc regions or fragments (e.g., C_{H}3 domains). As another non-limiting example, a knob can be designed on the MHC class II α chain or portion or fragment or variant thereof to be inserted into a corresponding hole on the MHC class II β chain or portion or fragment or variant thereof, or vice versa. Knobs were constructed by replacing amino acids with small side chains with amino acids with large side chains. Holes of identical or similar size to the knobs can be created by replacing amino acids with large side chains with amino acids with smaller ones, in this case. As a non-limiting example, a knob can be constructed by replacing an amino acid with a small side chain with a tyrosine or tryptophan, and a corresponding hole can be constructed by replacing an amino acid with a large side chain with an alanine or a threonine.

In another exemplary embodiment, the MHC class II α chain or portion or fragment or variant thereof and the MHC class II β chain or portion or fragment or variant thereof are linked based on charge mutations. Contact residues between the MHC class II α chain or portion or fragment or variant thereof and the MHC class II β chain or portion or fragment or variant thereof can be amino acids that are charged or amino acids that are neutral. A charged amino acid is an amino acid residue with an electrically charged side chain. These can either be positively charged side chains, such as present in arginine (Arg, R), histidine (His, H), and lysine (Lys, K) or can be negatively charged side chains, such as present in aspartic acid (Asp, D) and glutamic acid (Glu, E). A neutral amino acid is any other amino acid that does not carry an electrically charged side chain. These neutral residues include serine (Ser, S), threonine (Thr, T), asparagine (Asn, N), glutamine (Glu, Q), cysteine (Cys, C), glycine (Gly, G), proline (Pro, P), alanine (Ala, A), valine (Val, V), isoleucine (Ile, I), leucine (Leu, L), methionine (Met, M), phenylalanine (Phe, F), tyrosine (Tyr, Y), and tryptophan (Trp, T). As a non-limiting example, one or more positively charged amino acids can be engineered into the MHC class II α chain or portion or fragment or variant thereof to interact with one or more negatively charged amino acids in the MHC class II β chain or portion or fragment or variant thereof, or vice versa. As another non-limiting example, one or more negatively charged amino acids can be engineered into the MHC class II α chain or portion or fragment or variant thereof to interact with one or more positively charged amino acids in the MHC class II β chain or portion or fragment or variant thereof, or vice versa. As another non-limiting example, one or more negatively charged amino acids can be engineered into the MHC class II α chain or portion or fragment or variant thereof to interact with one or more positively charged amino acids engineered into the MHC class II β chain or portion or fragment or variant thereof, or vice versa. In some embodiments, a charged amino acid can be engineered into the MHC class II α chain or portion or fragment or variant thereof or the MHC class II β chain or portion or fragment or variant thereof by substituting a neutral amino acid residue with the charged amino acid residue. In some embodiments, a charged amino acid can be engineered into the MHC class II α chain or portion or fragment or variant thereof or the MHC class II β chain or portion or fragment or variant thereof by substituting an oppositely charged amino acid residue with the charged amino acid residue (e.g., substituting a negatively charged amino acid residue with a positively charged amino acid residue or substituting a positively charged amino acid residue with a negatively charged amino acid residue). In one embodiment, the MHC can be linked to immunoglobulin Fc regions that comprise differently charged mutations. See, e.g., U.S. Pat. No. 9,358,286, herein incorporated by reference in its entirety for all purposes.

In another embodiment, the MHC class II α chain or portion or fragment or variant thereof and the MHC class II β chain or portion or fragment or variant thereof are covalently linked (e.g., but not limited to, by a linker such as a peptide linker). See, e.g., Burrows et al. (1999) Protein Eng. 12(9):771-778, herein incorporated by reference in its entirety for all purposes. Such MHC class II molecules can be single-chain MHC fusions. In one exemplary embodiment, the single-chain MHC fusions can be minimal TCR binding units that comprise only the α1 and β1 domains or portions or fragments or variants thereof (or do not comprise the α2 and β2 domains and do not comprise transmembrane or cytoplasmic domains of the α and β chains). In an exemplary embodiment, the hydrophobic transmembrane regions of the MHC class II α chain and MHC class II β chain are replaced by a linker, such as a peptide linker. In one exemplary embodiment, the extracellular domain of the MHC class II α chain or a portion or fragment or variant thereof (e.g., but not limited to, comprising the MHC class II α1 domain or a portion or fragment or variant thereof or the MHC class II α1 and α2 domains or a portion or fragment or variant thereof) can be linked (e.g., but not limited to, fused in frame or via a linker) to the extracellular domain of the MHC class II β chain or a portion or fragment or variant thereof (e.g., but not limited to, comprising the MHC class II 1 domain or a portion or fragment or variant thereof or the MHC class II β1 and β2 domains or a portion or fragment or variant thereof). Suitable linkers are described elsewhere herein. In one exemplary embodiment, the N-terminal end of the α chain or a portion or fragment or variant thereof is linked to the C-terminal end of the β chain or a portion or fragment or variant thereof. In some embodiments, the C-terminal end of the α chain or a portion or fragment or variant thereof can be linked to the N-terminal end of the β chain or a portion or fragment or variant thereof. Suitable linkers are disclosed in more detail elsewhere herein.

B. MHC Ligand Peptides and Linkage to MHC Class II Molecules

The MHC ligand peptides in the peptide-MHC class II complexes may comprise, in some embodiments of the present invention, any peptide that is capable of binding to an MHC protein in a manner such that the MHC-peptide complex can bind to a T-cell receptor (TCR) and effect a T cell response (i.e., an antigenic peptide). The characteristics of antigenic peptides, such as the length, amino acid composition, and the like can depend on several factors, including, but not limited to, the ability of the peptide to fit within the peptide-binding groove, experimental conditions, and the antigen of interest. These factors can be determined through the use of commercially available computer programs, such as Protean II™ (Proteus) and SPOT™. The binding of a peptide to an MHC peptide-binding groove can control the spatial arrangement of MHC and/or peptide amino acid residues recognized by a TCR, or pMHC-binding protein produced by an animal. Such spatial control is due in part to hydrogen bonds formed between a peptide and an MHC protein. Based on the knowledge on how peptides bind to various MHCs, the major MHC anchor amino acids and the surface exposed amino acids that are varied among different peptides can be determined. Peptide binding to MHC class II molecules is stabilized by hydrophobic anchoring and hydrogen bond formation. The peptide adopts a type II polyproline helix as it interacts with the binding groove. Without wishing to be bound by theory, it is believed that this conformation causes the peptide to twist in a specific fashion, with the sequestration of peptide side chains in polymorphic pockets in the MHC II protein. See, e.g., Ferrante and Gorski (2007) J. Immunol. 178:7181-7189, herein incorporated by reference in its entirety for all purposes. Without wishing to be bound by theory, it is believed that generally these pockets accommodate the side chains of peptide residues at the P1, P4, P6, and P9 positions and have been identified as major anchors. In addition to these largely solvent inaccessible interactions, positions with smaller pockets or shelves in the binding site accommodating the P2, P3, P7, and P10 residues are recognized as minor or auxiliary anchors.

In some embodiments, non-limiting examples of MHC ligand peptides suitable for use in the disclosed peptide-MHC class II complexes include peptides comprising the P-3 to P12 residues. In some embodiments, non-limiting examples of MHC ligand peptides suitable for use in the disclosed peptide-MHC class II complexes include peptides consisting essentially of the P-3 to P12 residues. In some embodiments, non-limiting examples of MHC ligand peptides suitable for use in the disclosed peptide-MHC class II complexes include peptides consisting of the P-3 to P12 residues. In some embodiments, non-limiting examples of suitable MHC ligand peptides include peptides comprising the P-2 to P11 residues. In some embodiments, non-limiting examples of suitable MHC ligand peptides include peptides consisting essentially of the P-2 to P11 residues. In some embodiments, non-limiting examples of suitable MHC ligand peptides include peptides consisting of the P-2 to P11 residues. In some embodiments, non-limiting examples of suitable MHC ligand peptides include peptides comprising the P-1 to P10 residues. In some embodiments, non-limiting examples of suitable MHC ligand peptides include peptides consisting essentially of the P-1 to P10 residues. In some embodiments, non-limiting examples of suitable MHC ligand peptides include peptides consisting of the P-1 to P10 residues. In some embodiments, non-limiting examples of suitable MHC ligand peptides include peptides comprising the P1 to P9 residues. In some embodiments, non-limiting examples of suitable MHC ligand peptides include peptides consisting essentially of the P1 to P9 residues. In some embodiments, non-limiting examples of suitable MHC ligand peptides include peptides consisting of the P1 to P9 residues.

In some embodiments, non-limiting examples of suitable MHC ligand peptides include peptides comprising the P-3 to P12 residues. In some embodiments, non-limiting examples of suitable MHC ligand peptides include peptides comprising the P-2 to P12 residues. In some embodiments, non-limiting examples of suitable MHC ligand peptides include peptides comprising the P-1 to P12 residues. In some embodiments, non-limiting examples of suitable MHC ligand peptides include peptides comprising the P1 to P12 residues. In some embodiments, non-limiting examples of suitable MHC ligand peptides include peptides comprising the P-3 to P11 residues. In some embodiments, non-limiting examples of suitable MHC ligand peptides include peptides comprising the P-2 to P11 residues. In some embodiments, non-limiting examples of suitable MHC ligand peptides include peptides comprising the P-1 to P11 residues. In some embodiments, non-limiting examples of suitable MHC ligand peptides include peptides comprising the P1 to P11 residues. In some embodiments, non-limiting examples of suitable MHC ligand peptides include peptides comprising the P-3 to P10 residues. In some embodiments, non-limiting examples of suitable MHC ligand peptides include peptides comprising the P-2 to P10 residues. In some embodiments, non-limiting examples of suitable MHC ligand peptides include peptides comprising the P-1 to P10 residues. In some embodiments, non-limiting examples of suitable MHC ligand peptides include peptides comprising the P1 to P10 residues. In some embodiments, non-limiting examples of suitable MHC ligand peptides include peptides comprising the P-3 to P9 residues. In some embodiments, non-limiting examples of suitable MHC ligand peptides include peptides comprising the P-2 to P9 residues. In some embodiments, non-limiting examples of suitable MHC ligand peptides include peptides comprising the P-1 to P9 residues. In some embodiments, non-limiting examples of suitable MHC ligand peptides include peptides comprising the P1 to P9 residues.

In some embodiments, non-limiting examples of suitable MHC ligand peptides include peptides consisting essentially of the P-3 to P12 residues. In some embodiments, non-limiting examples of suitable MHC ligand peptides include peptides consisting essentially of the P-2 to P12 residues. In some embodiments, non-limiting examples of suitable MHC ligand peptides include peptides consisting essentially of the P-1 to P12 residues. In some embodiments, non-limiting examples of suitable MHC ligand peptides include peptides consisting essentially of the P1 to P12 residues. In some embodiments, non-limiting examples of suitable MHC ligand peptides include peptides consisting essentially of the P-3 to P11 residues. In some embodiments, non-limiting examples of suitable MHC ligand peptides include peptides consisting essentially of the P-2 to P11 residues. In some embodiments, non-limiting examples of suitable MHC ligand peptides include peptides consisting essentially of the P-1 to P11 residues. In some embodiments, non-limiting examples of suitable MHC ligand peptides include peptides consisting essentially of the P1 to P11 residues. In some embodiments, non-limiting examples of suitable MHC ligand peptides include peptides consisting essentially of the P-3 to P10 residues. In some embodiments, non-limiting examples of suitable MHC ligand peptides include peptides consisting essentially of the P-2 to P10 residues. In some embodiments, non-limiting examples of suitable MHC ligand peptides include peptides consisting essentially of the P-1 to P10 residues. In some embodiments, non-limiting examples of suitable MHC ligand peptides include peptides consisting essentially of the P1 to P10 residues. In some embodiments, non-limiting examples of suitable MHC ligand peptides include peptides consisting essentially of the P-3 to P9 residues. In some embodiments, non-limiting examples of suitable MHC ligand peptides include peptides consisting essentially of the P-2 to P9 residues. In some embodiments, non-limiting examples of suitable MHC ligand peptides include peptides consisting essentially of the P-1 to P9 residues. In some embodiments, non-limiting examples of suitable MHC ligand peptides include peptides consisting essentially of the P1 to P9 residues.

In some embodiments, non-limiting examples of suitable MHC ligand peptides include peptides consisting of the P-3 to P12 residues. In some embodiments, non-limiting examples of suitable MHC ligand peptides include peptides consisting of the P-2 to P12 residues. In some embodiments, non-limiting examples of suitable MHC ligand peptides include peptides consisting of the P-1 to P12 residues. In some embodiments, non-limiting examples of suitable MHC ligand peptides include peptides consisting of the P1 to P12 residues. In some embodiments, non-limiting examples of suitable MHC ligand peptides include peptides consisting of the P-3 to P11 residues. In some embodiments, non-limiting examples of suitable MHC ligand peptides include peptides consisting of the P-2 to P11 residues. In some embodiments, non-limiting examples of suitable MHC ligand peptides include peptides consisting of the P-1 to P11 residues. In some embodiments, non-limiting examples of suitable MHC ligand peptides include peptides consisting of the P1 to P11 residues. In some embodiments, non-limiting examples of suitable MHC ligand peptides include peptides consisting of the P-3 to P10 residues. In some embodiments, non-limiting examples of suitable MHC ligand peptides include peptides consisting of the P-2 to P10 residues. In some embodiments, non-limiting examples of suitable MHC ligand peptides include peptides consisting of the P-1 to P10 residues. In some embodiments, non-limiting examples of suitable MHC ligand peptides include peptides consisting of the P1 to P10 residues. In some embodiments, non-limiting examples of suitable MHC ligand peptides include peptides consisting of the P-3 to P9 residues. In some embodiments, non-limiting examples of suitable MHC ligand peptides include peptides consisting of the P-2 to P9 residues. In some embodiments, non-limiting examples of suitable MHC ligand peptides include peptides consisting of the P-1 to P9 residues. In some embodiments, non-limiting examples of suitable MHC ligand peptides include peptides consisting of the P1 to P9 residues.

In some embodiments, non-limiting examples of suitable MHC ligand peptides include peptides comprising the P-3 to P12 residues. In some embodiments, non-limiting examples of suitable MHC ligand peptides include peptides comprising the P-3 to P11 residues. In some embodiments, non-limiting examples of suitable MHC ligand peptides include peptides comprising the P-3 to P10 residues. In some embodiments, non-limiting examples of suitable MHC ligand peptides include peptides comprising the P-3 to P9 residues. In some embodiments, non-limiting examples of suitable MHC ligand peptides include peptides comprising the P-2 to P12 residues. In some embodiments, non-limiting examples of suitable MHC ligand peptides include peptides comprising the P-2 to P11 residues. In some embodiments, non-limiting examples of suitable MHC ligand peptides include peptides comprising the P-2 to P10 residues. In some embodiments, non-limiting examples of suitable MHC ligand peptides include peptides comprising the P-2 to P9 residues. In some embodiments, non-limiting examples of suitable MHC ligand peptides include peptides comprising the P-1 to P12 residues. In some embodiments, non-limiting examples of suitable MHC ligand peptides include peptides comprising the P-1 to P11 residues. In some embodiments, non-limiting examples of suitable MHC ligand peptides include peptides comprising the P-1 to P10 residues. In some embodiments, non-limiting examples of suitable MHC ligand peptides include peptides comprising the P-1 to P9 residues. In some embodiments, non-limiting examples of suitable MHC ligand peptides include peptides comprising the P1 to P12 residues. In some embodiments, non-limiting examples of suitable MHC ligand peptides include peptides comprising the P1 to P11 residues. In some embodiments, non-limiting examples of suitable MHC ligand peptides include peptides comprising the P1 to P10 residues. In some embodiments, non-limiting examples of suitable MHC ligand peptides include peptides comprising the P1 to P9 residues.

In some embodiments, non-limiting examples of suitable MHC ligand peptides include peptides consisting essentially of the P-3 to P12 residues. In some embodiments, non-limiting examples of suitable MHC ligand peptides include peptides consisting essentially of the P-3 to P11 residues. In some embodiments, non-limiting examples of suitable MHC ligand peptides include peptides consisting essentially of the P-3 to P10 residues. In some embodiments, non-limiting examples of suitable MHC ligand peptides include peptides consisting essentially of the P-3 to P9 residues. In some embodiments, non-limiting examples of suitable MHC ligand peptides include peptides consisting essentially of the P-2 to P12 residues. In some embodiments, non-limiting examples of suitable MHC ligand peptides include peptides consisting essentially of the P-2 to P11 residues. In some embodiments, non-limiting examples of suitable MHC ligand peptides include peptides consisting essentially of the P-2 to P10 residues. In some embodiments, non-limiting examples of suitable MHC ligand peptides include peptides consisting essentially of the P-2 to P9 residues. In some embodiments, non-limiting examples of suitable MHC ligand peptides include peptides consisting essentially of the P-1 to P12 residues. In some embodiments, non-limiting examples of suitable MHC ligand peptides include peptides consisting essentially of the P-1 to P11 residues. In some embodiments, non-limiting examples of suitable MHC ligand peptides include peptides consisting essentially of the P-1 to P10 residues. In some embodiments, non-limiting examples of suitable MHC ligand peptides include peptides consisting essentially of the P-1 to P9 residues. In some embodiments, non-limiting examples of suitable MHC ligand peptides include peptides consisting essentially of the P1 to P12 residues. In some embodiments, non-limiting examples of suitable MHC ligand peptides include peptides consisting essentially of the P1 to P11 residues. In some embodiments, non-limiting examples of suitable MHC ligand peptides include peptides consisting essentially of the P1 to P10 residues. In some embodiments, non-limiting examples of suitable MHC ligand peptides include peptides consisting essentially of the P1 to P9 residues.

In some embodiments, non-limiting examples of suitable MHC ligand peptides include peptides consisting of the P-3 to P12 residues. In some embodiments, non-limiting examples of suitable MHC ligand peptides include peptides consisting of the P-3 to P11 residues. In some embodiments, non-limiting examples of suitable MHC ligand peptides include peptides consisting of the P-3 to P10 residues. In some embodiments, non-limiting examples of suitable MHC ligand peptides include peptides consisting of the P-3 to P9 residues. In some embodiments, non-limiting examples of suitable MHC ligand peptides include peptides consisting of the P-2 to P12 residues. In some embodiments, non-limiting examples of suitable MHC ligand peptides include peptides consisting of the P-2 to P11 residues. In some embodiments, non-limiting examples of suitable MHC ligand peptides include peptides consisting of the P-2 to P10 residues. In some embodiments, non-limiting examples of suitable MHC ligand peptides include peptides consisting of the P-2 to P9 residues. In some embodiments, non-limiting examples of suitable MHC ligand peptides include peptides consisting of the P-1 to P12 residues. In some embodiments, non-limiting examples of suitable MHC ligand peptides include peptides consisting of the P-1 to P11 residues. In some embodiments, non-limiting examples of suitable MHC ligand peptides include peptides consisting of the P-1 to P10 residues. In some embodiments, non-limiting examples of suitable MHC ligand peptides include peptides consisting of the P-1 to P9 residues. In some embodiments, non-limiting examples of suitable MHC ligand peptides include peptides consisting of the P1 to P12 residues. In some embodiments, non-limiting examples of suitable MHC ligand peptides include peptides consisting of the P1 to P11 residues. In some embodiments, non-limiting examples of suitable MHC ligand peptides include peptides consisting of the P1 to P10 residues. In some embodiments, non-limiting examples of suitable MHC ligand peptides include peptides consisting of the P1 to P9 residues.

In one embodiment, an MHC ligand peptide comprises the P1 to P12 residues. In one embodiment, an MHC ligand peptide consists essentially of the P1 to P12 residues. In one embodiment, an MHC ligand peptide consists of the P1 to P12 residues. In one embodiment, an MHC ligand peptide comprises the P-1 to P11 residues. In one embodiment, an MHC ligand peptide consists essentially of the P-1 to P11 residues. In one embodiment, an MHC ligand peptide consists of the P-1 to P11 residues. In one embodiment, an MHC ligand peptide comprises the P-1 to P9 residues. In one embodiment, an MHC ligand peptide consists essentially of the P-1 to P9 residues. In one embodiment, an MHC ligand peptide consists of the P-1 to P9 residues. In one embodiment, an MHC ligand peptide comprises the P-3 to P9 residues. In one embodiment, an MHC ligand peptide consists essentially of the P-3 to P9 residues. In one embodiment, an MHC ligand peptide consists of the P-3 to P9 residues.

In some embodiments, non-limiting examples of antigenic peptides suitable for use in the disclosed peptide-MHC class II complexes include peptides comprising an antigen or a portion or fragment or variant thereof selected from a group consisting of autoantigens, tumor-associated antigens, infectious agents, toxins, allergens, or combinations thereof. In one exemplary embodiment, the MHC ligand peptide comprises at least a portion or fragment or variant (e.g., but not limited to, an antigenic determinant) of a human self-protein associated with an autoimmune disorder. In another embodiment, the MHC ligand peptide comprises at least a portion or fragment or variant (e.g., but not limited to, an antigenic determinant) of a protein of an infectious agent (e.g., but not limited to, bacterial, viral, or parasitic organisms). In another embodiment, the MHC ligand peptide comprises at least a portion or fragment or variant (e.g., but not limited to, an antigenic determinant) of an allergen. In another embodiment, the MHC ligand peptide comprises at least a portion or fragment or variant (e.g., but not limited to, an antigenic determinant) of a tumor-associated protein. In another embodiment, the MHC ligand peptide is associated with a T-cell-mediated disease (e.g., but not limited to, T-cell-mediated autoimmune disease such as type 1 diabetes mellitus, rheumatoid arthritis, multiple sclerosis, celiac disease, Addison's disease, and hypothyroidism). In one embodiment, the MHC ligand peptide can be a gliadin peptide or a gliadin-derived peptide. In another embodiment, the MHC ligand peptide (e.g., the gliadin peptide or gliadin-derived peptide) can comprise QLQPFPQPELPY (SEQ ID NO: 44), PQPELPYPQPQL (SEQ ID NO: 46), or FPQPEQPFPWQP (SEQ ID NO: 45). In another embodiment, the MHC ligand peptide (e.g., the gliadin peptide or gliadin-derived peptide) can consist essentially of QLQPFPQPELPY (SEQ ID NO: 44), PQPELPYPQPQL (SEQ ID NO: 46), or FPQPEQPFPWQP (SEQ ID NO: 45). In another embodiment, the MHC ligand peptide (e.g., the gliadin peptide or gliadin-derived peptide) can consist of QLQPFPQPELPY (SEQ ID NO: 44), PQPELPYPQPQL (SEQ ID NO: 46), or FPQPEQPFPWQP (SEQ ID NO: 45). In another embodiment, the MHC ligand peptide (e.g., the gliadin peptide or gliadin-derived peptide) can comprise QLQPFPQPELPY (SEQ ID NO: 44), PQPELPYPQPQL (SEQ ID NO: 46), FPQPEQPFPWQP (SEQ ID NO: 45), QPFPQPELPYPQ (SEQ ID NO: 69), QPFPQPEQPFPW (SEQ ID NO: 70), QPFPQPELPY (SEQ ID NO: 71), FPQPELPYPQ (SEQ ID NO: 72), or FPQPEQPFPW (SEQ ID NO: 73). In another embodiment, the MHC ligand peptide (e.g., the gliadin peptide or gliadin-derived peptide) can consist essentially of QLQPFPQPELPY (SEQ ID NO: 44), PQPELPYPQPQL (SEQ ID NO: 46), FPQPEQPFPWQP (SEQ ID NO: 45), QPFPQPELPYPQ (SEQ ID NO: 69), QPFPQPEQPFPW (SEQ ID NO: 70), QPFPQPELPY (SEQ ID NO: 71), FPQPELPYPQ (SEQ ID NO: 72), or FPQPEQPFPW (SEQ ID NO: 73). In another embodiment, the MHC ligand peptide (e.g., the gliadin peptide or gliadin-derived peptide) can consist of QLQPFPQPELPY (SEQ ID NO: 44), PQPELPYPQPQL (SEQ ID NO: 46), FPQPEQPFPWQP (SEQ ID NO: 45), QPFPQPELPYPQ (SEQ ID NO: 69), QPFPQPEQPFPW (SEQ ID NO: 70), QPFPQPELPY (SEQ ID NO: 71), FPQPELPYPQ (SEQ ID NO: 72), or FPQPEQPFPW (SEQ ID NO: 73).

In some embodiments, MHC ligand peptides can be any suitable length for binding to an MHC protein in a manner such that the MHC-peptide complex can bind to a TCR and effect a T cell response. MHC ligand peptide length can vary, for example, from about 5 to about 40 amino acids (e.g., but not limited to, from about 6 to about 30 amino acids, from about 8 to about 20 amino acids, from about 10 to about 18 amino acids, from about 12 to about 18 amino acids, from about 13 to about 18 amino acids, from about 9 to about 11 amino acids, or any size peptide between 5 and 40 amino acids in length, in whole integer increments (i.e., 5, 6, 7, 8, 9 . . . 40)). While naturally MHC-class-II-bound peptides vary from about 9 to about 40 amino acids, in nearly all cases the peptide can be truncated to a 9-11 amino acid core without loss of MHC-binding activity or T-cell recognition. In some embodiments, the MHC ligand peptide length can be from about 5 to about 40 amino acids. In some embodiments, the MHC ligand peptide length can be from about 10 to about 40 amino acids. In some embodiments, the MHC ligand peptide length can be from about 15 to about 40 amino acids. In some embodiments, the MHC ligand peptide length can be from about 20 to about 40 amino acids. In some embodiments, the MHC ligand peptide length can be from about 25 to about 40 amino acids. In some embodiments, the MHC ligand peptide length can be from about 30 to about 40 amino acids. In some embodiments, the MHC ligand peptide length can be from about 35 to about 40 amino acids. In some embodiments, the MHC ligand peptide length can be from about 5 to about 35 amino acids. In some embodiments, the MHC ligand peptide length can be from about 5 to about 30 amino acids. In some embodiments, the MHC ligand peptide length can be from about 5 to about 25 amino acids. In some embodiments, the MHC ligand peptide length can be from about 5 to about 20 amino acids. In some embodiments, the MHC ligand peptide length can be from about 5 to about 15 amino acids. In some embodiments, the MHC ligand peptide length can be from about 5 to about 10 amino acids. In some embodiments, the MHC ligand peptide length can be from about 10 to about 35 amino acids. In some embodiments, the MHC ligand peptide length can be from about 15 to about 30 amino acids. In some embodiments, the MHC ligand peptide length can be from about 20 to about 25 amino acids. In some embodiments, the MHC ligand peptide length can be from about 9 to about 11 amino acids. In some embodiments, the MHC ligand peptide length can be from about 10 to about 18 amino acids. In some embodiments, the MHC ligand peptide can be from about 9 to about 15 amino acids. In some embodiments, the MHC ligand peptide can be from about 9 to about 14 amino acids. In some embodiments, the MHC ligand peptide can be from about 9 to about 13 amino acids. In some embodiments, the MHC ligand peptide can be from about 9 to about 12 amino acids. In some embodiments, the MHC ligand peptide can be from about 10 to about 15 amino acids. In some embodiments, the MHC ligand peptide can be from about 10 to about 14 amino acids. In some embodiments, the MHC ligand peptide can be from about 10 to about 13 amino acids. In some embodiments, the MHC ligand peptide can be from about 10 to about 12 amino acids.

In some embodiments, MHC ligand peptides can be any suitable length for binding to an MHC protein in a manner such that the MHC-peptide complex can bind to a TCR and effect a T cell response. MHC ligand peptide length can vary, for example, from 5 to 40 amino acids (e.g., but not limited to, from 6 to 30 amino acids, from 8 to 20 amino acids, from 10 to 18 amino acids, from 12 to 18 amino acids, from 13 to 18 amino acids, from 9 to 11 amino acids, or any size peptide between 5 and 40 amino acids in length, in whole integer increments (i.e., 5, 6, 7, 8, 9 . . . 40)). While naturally MHC-class-II-bound peptides vary from 9 to 40 amino acids, in nearly all cases the peptide can be truncated to a 9-11 amino acid core without loss of MHC-binding activity or T-cell recognition. In some embodiments, the MHC ligand peptide length can be from 5 to 40 amino acids. In some embodiments, the MHC ligand peptide length can be from 10 to 40 amino acids. In some embodiments, the MHC ligand peptide length can be from 15 to 40 amino acids. In some embodiments, the MHC ligand peptide length can be from 20 to 40 amino acids. In some embodiments, the MHC ligand peptide length can be from 25 to 40 amino acids. In some embodiments, the MHC ligand peptide length can be from 30 to 40 amino acids. In some embodiments, the MHC ligand peptide length can be from 35 to 40 amino acids. In some embodiments, the MHC ligand peptide length can be from 5 to 35 amino acids. In some embodiments, the MHC ligand peptide length can be from 5 to 30 amino acids. In some embodiments, the MHC ligand peptide length can be from 5 to 25 amino acids. In some embodiments, the MHC ligand peptide length can be from 5 to 20 amino acids. In some embodiments, the MHC ligand peptide length can be from 5 to 15 amino acids. In some embodiments, the MHC ligand peptide length can be from 5 to 10 amino acids. In some embodiments, the MHC ligand peptide length can be from 10 to 35 amino acids. In some embodiments, the MHC ligand peptide length can be from 15 to 30 amino acids. In some embodiments, the MHC ligand peptide length can be from 20 to 25 amino acids. In some embodiments, the MHC ligand peptide length can be from 9 to 11 amino acids. In some embodiments, the MHC ligand peptide length can be from 10 to 18 amino acids. In some embodiments, the MHC ligand peptide can be from 9 to 15 amino acids. In some embodiments, the MHC ligand peptide can be from 9 to 14 amino acids. In some embodiments, the MHC ligand peptide can be from 9 to 13 amino acids. In some embodiments, the MHC ligand peptide can be from 9 to 12 amino acids. In some embodiments, the MHC ligand peptide can be from 10 to 15 amino acids. In some embodiments, the MHC ligand peptide can be from 10 to 14 amino acids. In some embodiments, the MHC ligand peptide can be from 10 to 13 amino acids. In some embodiments, the MHC ligand peptide can be from 10 to 12 amino acids.

(1) Linkers

In some embodiments of complexes, at least one chain or portion or fragment or variant thereof of the MHC class II molecule and the MHC ligand peptide are associated as a fusion protein. In an exemplary embodiment, the MHC class II molecule (e.g., but not limited to, an MHC class II 3 chain or portion or fragment or variant thereof or an MHC class II α chain or portion or fragment or variant thereof) and the MHC ligand peptide can be connected via a linker (e.g., but not limited to, covalently linked, such as by a peptide linker). As a non-limiting example, the MHC ligand peptide can be connected directly or indirectly to the N-terminal end of the MHC class II 3 chain or portion or fragment or variant thereof, to the C-terminal end of the MHC class II 3 chain or portion or fragment or variant thereof, to the N-terminal end of the MHC class II α chain or portion or fragment or variant thereof, or to the C-terminal end of the MHC class II α chain or portion or fragment or variant thereof. In an exemplary embodiment, the MHC ligand peptide can be connected directly or indirectly to the N-terminal end of the MHC class II 3 chain or portion or fragment or variant thereof. As a non-limiting example, the peptide-MHC class II complex can comprise from amino to carboxy terminal the MHC ligand peptide, a linker, and an MHC class II 3 chain or portion or fragment or variant thereof. As a non-limiting example, the linker can extend from the C-terminal end of the MHC ligand peptide to the N-terminal end of the MHC class II 3 chain or portion or fragment or variant thereof. The linker can be structured to allow the linked MHC ligand peptide to fold into the binding groove of the MHC class II molecule, resulting in a functional peptide-MHC class II complex. Attaching the peptide to the MHC class II molecule via a flexible linker has the advantage of assuring that the peptide will occupy and stay associated with the MHC during biosynthesis, transport, and display.

The length of a linker connecting the MHC ligand peptide to the MHC class II molecule can be any suitable length. In an exemplary embodiment, the linker can be sufficiently long that the MHC ligand peptide can reach and bind to the peptide-binding groove of the MHC class II molecule and sufficiently short such that the linker does not substantially inhibit binding between the MHC ligand peptide and the peptide-binding groove of the MHC class II molecule. Linker length can be designed to span in excess of the distance between the N-terminus and C-terminus being joined based on known structural information (e.g., but not limited to, known tertiary structure information). Suitable size and sequences of linkers also can be determined by conventional computer modeling techniques based on the predicted tertiary structure of the peptide-MHC class II complex. As a non-limiting example, from the HLA-DQ structure deposited as PDB code 1S9V, the length between the C-alpha atom of the MHC ligand peptide C-terminus and C-alpha atom of the N-terminus of either the MHC a subunit or the MHC R subunit is about 30 Å. See, e.g., Kim et al. (2004) Proc. Natl. Acad. Sci. U.S.A. 101(12):4175-4179, herein incorporated by reference in its entirety for all purposes. Thus, if the C-terminal end of the MHC ligand peptide is connected via a linker to the N-terminal end of the MHC class II β chain or portion or domain or fragment or variant thereof or the MHC class II α chain or portion or domain or fragment or variant thereof, the length of the linker can be designed to surpass the distance between the C-terminal end of the MHC ligand peptide and the N-terminal end of the MHC class II α or β chain or portion or domain or fragment or variant thereof when the MHC ligand peptide is positioned within the peptide-binding groove of the MHC class II molecule. From the above measurement, for example, a linker in excess of 30 Å (3.5 Å per amino acid residue=9 amino acids) may be required to span the measured distance. Additional amino acids could also be included so that the linker can avoid the protein molecular surface between the two connection points.

As a non-limiting example, a linker can be at least about 9 amino acids, at least about 10 amino acids, at least about 11 amino acids, at least about 12 amino acids, at least about 13 amino acids, at least about 14 amino acids, or at least about 15 amino acids in length. Likewise, in some embodiments, a linker can be no more than about 50 amino acids, no more than about 45 amino acids, no more than about 40 amino acids, no more than about 35 amino acids, no more than about 30 amino acids, no more than about 25 amino acids, no more than about 20 amino acids, or no more than about 15 amino acids in length. In some embodiments, a linker can be between about 9 amino acids and about 50 amino acids in length. In some embodiments, a linker can be between about 9 amino acids and about 45 amino acids in length. In some embodiments, a linker can be between about 9 amino acids and about 40 amino acids in length. In some embodiments, a linker can be between about 9 amino acids and about 35 amino acids in length. In some embodiments, a linker can be between about 9 amino acids and about 30 amino acids in length. In some embodiments, a linker can be between about 9 amino acids and about 25 amino acids in length. In some embodiments, a linker can be between about 9 amino acids and about 20 amino acids in length. In some embodiments, a linker can be between about 10 amino acids and about 45 amino acids in length. In some embodiments, a linker can be between about 10 amino acids and about 40 amino acids in length. In some embodiments, a linker can be between about 10 amino acids and about 35 amino acids in length. In some embodiments, a linker can be between about 10 amino acids and about 30 amino acids in length. In some embodiments, a linker can be between about 10 amino acids and about 25 amino acids in length. In some embodiments, a linker can be between about 10 amino acids and about 20 amino acids in length. In some embodiments, a linker can be between about 15 amino acids and about 45 amino acids in length. In some embodiments, a linker can be between about 15 amino acids and about 40 amino acids in length. In some embodiments, a linker can be between about 15 amino acids and about 35 amino acids in length. In some embodiments, a linker can be between about 15 amino acids and about 30 amino acids in length. In some embodiments, a linker can be between about 15 amino acids and about 25 amino acids in length. In some embodiments, a linker can be between about 15 amino acids and about 20 amino acids in length. In some embodiments, a linker can be between about 10 amino acids and about 50 amino acids in length. In some embodiments, a linker can be between about 15 amino acids and about 50 amino acids in length. In some embodiments, a linker can be between about 20 amino acids and about 50 amino acids in length. In some embodiments, a linker can be between about 25 amino acids and about 50 amino acids in length. In some embodiments, a linker can be between about 30 amino acids and about 50 amino acids in length. In some embodiments, a linker can be between about 35 amino acids and about 50 amino acids in length. In some embodiments, a linker can be between about 40 amino acids and about 50 amino acids in length. In some embodiments, a linker can be between about 45 amino acids and about 50 amino acids in length. In some embodiments, a linker can be between about 10 amino acids and about 20 amino acids in length. In some embodiments, a linker can be between about 11 amino acids and about 19 amino acids in length. In some embodiments, a linker can be between about 12 amino acids and about 18 amino acids in length. In some embodiments, a linker can be between about 13 amino acids and about 17 amino acids in length. In some embodiments, a linker can be between about 14 amino acids and about 16 amino acids in length. In some embodiments, a linker can be any size peptide between 9 and 50 amino acids in length, in whole integer increments (i.e., 9, 10, 11, 12, 13 . . . 50). In an exemplary embodiment, the linker can be about 15 amino acids in length.

As a non-limiting example, a linker can be at least 9 amino acids, at least 10 amino acids, at least 11 amino acids, at least 12 amino acids, at least 13 amino acids, at least 14 amino acids, or at least 15 amino acids in length. Likewise, in some embodiments, a linker can be no more than 50 amino acids, no more than 45 amino acids, no more than 40 amino acids, no more than 35 amino acids, no more than 30 amino acids, no more than 25 amino acids, no more than 20 amino acids, or no more than 15 amino acids in length. In some embodiments, a linker can be between 9 amino acids and 50 amino acids in length. In some embodiments, a linker can be between 9 amino acids and 45 amino acids in length. In some embodiments, a linker can be between 9 amino acids and 40 amino acids in length. In some embodiments, a linker can be between 9 amino acids and 35 amino acids in length. In some embodiments, a linker can be between 9 amino acids and 30 amino acids in length. In some embodiments, a linker can be between 9 amino acids and 25 amino acids in length. In some embodiments, a linker can be between 9 amino acids and 20 amino acids in length. In some embodiments, a linker can be between 10 amino acids and 45 amino acids in length. In some embodiments, a linker can be between 10 amino acids and 40 amino acids in length. In some embodiments, a linker can be between 10 amino acids and 35 amino acids in length. In some embodiments, a linker can be between 10 amino acids and 30 amino acids in length. In some embodiments, a linker can be between 10 amino acids and 25 amino acids in length. In some embodiments, a linker can be between 10 amino acids and 20 amino acids in length. In some embodiments, a linker can be between 15 amino acids and 45 amino acids in length. In some embodiments, a linker can be between 15 amino acids and 40 amino acids in length. In some embodiments, a linker can be between 15 amino acids and 35 amino acids in length. In some embodiments, a linker can be between 15 amino acids and 30 amino acids in length. In some embodiments, a linker can be between 15 amino acids and 25 amino acids in length. In some embodiments, a linker can be between 15 amino acids and 20 amino acids in length. In some embodiments, a linker can be between 10 amino acids and 50 amino acids in length. In some embodiments, a linker can be between 15 amino acids and 50 amino acids in length. In some embodiments, a linker can be between 20 amino acids and 50 amino acids in length. In some embodiments, a linker can be between 25 amino acids and 50 amino acids in length. In some embodiments, a linker can be between 30 amino acids and 50 amino acids in length. In some embodiments, a linker can be between 35 amino acids and 50 amino acids in length. In some embodiments, a linker can be between 40 amino acids and 50 amino acids in length. In some embodiments, a linker can be between 45 amino acids and 50 amino acids in length. In some embodiments, a linker can be between 10 amino acids and 20 amino acids in length. In some embodiments, a linker can be between 11 amino acids and 19 amino acids in length. In some embodiments, a linker can be between 12 amino acids and 18 amino acids in length. In some embodiments, a linker can be between 13 amino acids and 17 amino acids in length. In some embodiments, a linker can be between 14 amino acids and 16 amino acids in length. In some embodiments, a linker can be any size peptide between 9 and 50 amino acids in length, in whole integer increments (i.e., 9, 10, 11, 12, 13 . . . 50). In an exemplary embodiment, the linker can be 15 amino acids in length.

Any suitable amino acids can be used in a linker. In some embodiments, non-limiting examples of suitable linkers including flexible linkers, rigid linkers, and cleavable linkers are reviewed, for example, in Chen et al. (2013) Adv. Drug Deliv. Rev. 65(10):1357-1369, herein incorporated by reference in its entirety for all purposes. The PDB database can also be searched for amino acid sequences able to span defined distances between protein segments or domains, as demonstrated at the server from the Center for Integrative Bioinformatics, University of Amsterdam at the following site ibi.vu.nl/programs/linkerdbwww.

Linkers used in the peptide-MHC class II complexes disclosed in some embodiments herein can have one or more or all of the following features: the linkers are flexible, the linkers are non-immunogenic, the linkers do not include charged amino acids, the linkers include polar amino acids, and any combination thereof. Flexibility can allow the MHC ligand peptide to freely bind and assemble into its natural peptide-binding groove in the MHC class II molecule. Flexibility can be achieved, for example, by using linkers rich in small or hydrophilic amino acids (e.g., but not limited to, glycine and serine). In some embodiments, at least about 40%, at least about 45%, at least about 50%, at least about 55%, at least about 60%, at least about 65%, at least about 70%, at least about 75%, or at least about 80% of the amino acids in the linker can be glycines. In some embodiments, at least 40%, at least 45%, at least 50%, at least 55%, at least 60%, at least 65%, at least 70%, at least 75%, or at least 80% of the amino acids in the linker can be glycines. In some embodiments, between about 40% and about 80% of the amino acids in the linker can be glycines. In some embodiments, between 40% and 80% of the amino acids in the linker can be glycines. In some embodiments, between about 50% and about 80% of the amino acids in the linker can be glycines. In some embodiments, between 50% and 80% of the amino acids in the linker can be glycines. In some embodiments, between about 60% and about 80% of the amino acids in the linker can be glycines. In some embodiments, between 60% and 80% of the amino acids in the linker can be glycines. In some embodiments, between about 70% and about 80% of the amino acids in the linker can be glycines. In some embodiments, between 70% and 80% of the amino acids in the linker can be glycines. In some embodiments, between about 40% and about 70% of the amino acids in the linker can be glycines. In some embodiments, between 40% and 70% of the amino acids in the linker can be glycines. In some embodiments, between about 40% and about 60% of the amino acids in the linker can be glycines. In some embodiments, between 40% and 60% of the amino acids in the linker can be glycines. In some embodiments, between about 40% and about 50% of the amino acids in the linker can be glycines. In some embodiments, between 40% and 50% of the amino acids in the linker can be glycines. In some embodiments, between about 50% and about 70% of the amino acids in the linker can be glycines. In some embodiments, between 50% and 70% of the amino acids in the linker can be glycines. In some embodiments, between about 55% and about 65% of the amino acids in the linker can be glycines. In some embodiments, between 55% and 65% of the amino acids in the linker can be glycines. Including polar amino acids can help improve solubility. In some embodiments, non-limiting examples of polar amino acids include Arg, Asn, Asp, Glu, Gln, His, Lys, Ser, Thr, and Tyr. In an exemplary embodiment, one or more serines are included in the linker. Omitting charged amino acids (e.g., but not limited to, Lys, Arg, Glu, and Asp) can help avoid electrostatic interactions with other amino acid side chains. In some embodiments of the peptide-MHC class II complexes disclosed herein, the linkers are flexible and include one or more polar amino acids. In some embodiments of the peptide-MHC class II complexes disclosed herein, the linkers are flexible (e.g., but not limited to, include flexible amino acids such as Gly) and do not include any charged amino acids. In some embodiments of the peptide-MHC class II complexes disclosed herein, the linkers are flexible and non-immunogenic. In some embodiments of the peptide-MHC class II complexes disclosed herein, the linkers include one or more polar amino acids and do not include any charged amino acids. In some embodiments of the peptide-MHC class II complexes disclosed herein, the linkers are non-immunogenic and include one or more polar amino acids. In some embodiments of the peptide-MHC class II complexes disclosed herein, the linkers are non-immunogenic and do not include any charged amino acids. In some embodiments of the peptide-MHC class II complexes disclosed herein, the linkers are flexible, include one or more polar amino acids, and do not include any charged amino acids. In some embodiments of the peptide-MHC class II complexes disclosed herein, the linkers are non-immunogenic, include one or more polar amino acids, and do not include any charged amino acids. In some embodiments of the peptide-MHC class II complexes disclosed herein, the linkers are flexible, non-immunogenic, and include one or more polar amino acids. In some embodiments of the peptide-MHC class II complexes disclosed herein, the linkers are flexible, non-immunogenic, and do not include any charged amino acids. In some embodiments of the peptide-MHC class II complexes disclosed herein, the linkers are flexible, non-immunogenic, include one or more polar amino acids, and do not include any charged amino acids.

In some embodiments, linkers suitable for use in the disclosed peptide-MHC class II complexes are cleavable linkers comprising a cleavage site. As a non-limiting example, a linker can comprise a tobacco etch virus (TEV) protease cleavage site ENLYFQ (SEQ ID NO: 22). However, other cleavage sites can also be used (e.g., but not limited to, a thrombin-sensitive cleavage site, a furin-sensitive cleavage site, a rhinovirus 3C protease cleavage site, or an enteropeptidase cleavage site). See, e.g., Waugh (2011) Protein Expr. Purif 80(2):283-293, herein incorporated by reference in its entirety for all purposes.

Some linkers suitable for use in the disclosed peptide-MHC class II complexes predominantly comprise amino acids with small side chains, such as glycine, alanine, and serine (e.g., but not limited to, glycine and serine). In some embodiments, exemplary linkers include glycine polymers (G)ₙ, glycine-serine polymers (including, for example, (GS)ₙ, (GSGGS)ₙ(SEQ ID NO: 2), (GGGS)ₙ (SEQ ID NO: 3), and (GGGGS)ₙ (SEQ ID NO: 4), where n is an integer of at least one), glycine-alanine polymers, alanine-serine polymers, and other flexible linkers that are well-known. The (GGGGS)ₙ (SEQ ID NO: 4) linker is particularly suitable, as it includes both a flexible amino acid (Gly) and a polar amino acid able to form hydrogen bonds (Ser), which improves solubility. Suitable linkers can comprise any of (GSGGS)ₙ(SEQ ID NO: 2), (GGGS)ₙ(SEQ ID NO: 3), and (GGGGS)ₙ(SEQ ID NO: 4). Suitable linkers can consist essentially of any of (GSGGS)ₙ (SEQ ID NO: 2), (GGGS)ₙ(SEQ ID NO: 3), and (GGGGS)ₙ (SEQ ID NO: 4). Suitable linkers can consist of any of (GSGGS)ₙ (SEQ ID NO: 2), (GGGS)ₙ (SEQ ID NO: 3), and (GGGGS)ₙ (SEQ ID NO: 4). In some embodiments, a peptide linker (e.g., a peptide linker linking the MHC ligand peptide to the MHC class II molecule) can comprise about 2 to about 4 repeats of the sequence set forth in SEQ ID NO: 4. In some embodiments, a peptide linker (e.g., a peptide linker linking the MHC ligand peptide to the MHC class II molecule) can comprise 2 to 4 repeats of the sequence set forth in SEQ ID NO: 4. In some embodiments, a peptide linker (e.g., a peptide linker linking the MHC ligand peptide to the MHC class II molecule) can comprise about 2 to about 4 repeats of the sequence set forth in SEQ ID NO: 4, wherein one amino acid in one of the repeats is mutated to cysteine. In some embodiments, a peptide linker (e.g., a peptide linker linking the MHC ligand peptide to the MHC class II molecule) can comprise 2 to 4 repeats of the sequence set forth in SEQ ID NO: 4, wherein one amino acid in one of the repeats is mutated to cysteine. As a non-limiting example, the cysteine can be the first, second, third, or fourth amino acid of the linker (e.g., but not limited to, the second amino acid of the linker). Glycine and glycine-serine polymers can be used. Both glycine and serine are relatively unstructured and therefore can serve as a neutral tether between components. Glycine accesses significantly more phi-psi space than even alanine and is much less restricted than residues with longer side chains. In some embodiments, exemplary linkers can comprise amino acid sequences including, but not limited to, GGSG (SEQ ID NO: 5), GGSGG (SEQ ID NO: 6), GSGSG (SEQ ID NO: 7), GSGGG (SEQ ID NO: 8), GGGSG (SEQ ID NO: 9), GSSSG (SEQ ID NO: 10), SGGGGG (SEQ ID NO: 11), GCGASGGGGSGGGGS (SEQ ID NO: 12), GCGASGGGGSGGGGS (SEQ ID NO: 13), GGGGSGGGGS (SEQ ID NO: 14), GGGASGGGGSGGGGS (SEQ ID NO: 15), GGGGSGGGGSGGGGS (SEQ ID NO: 16), or GGGASGGGGS (SEQ ID NO: 17), GGGGSGGGGSGGGGS (SEQ ID NO: 18), GGGGSGGGGSGGGGSGGGGS (SEQ ID NO: 19), GCGGS (SEQ ID NO: 20), GCGGSGGGGSGGGGS (SEQ ID NO: 21), GGGGSENLYFQGGGGS (SEQ ID NO: 47), and the like. In some embodiments, exemplary linkers can consist essentially of amino acid sequences including, but not limited to, GGSG (SEQ ID NO: 5), GGSGG (SEQ ID NO: 6), GSGSG (SEQ ID NO: 7), GSGGG (SEQ ID NO: 8), GGGSG (SEQ ID NO: 9), GSSSG (SEQ ID NO: 10), SGGGGG (SEQ ID NO: 11), GCGASGGGGSGGGGS (SEQ ID NO: 12), GCGASGGGGSGGGGS (SEQ ID NO: 13), GGGGSGGGGS (SEQ ID NO: 14), GGGASGGGGSGGGGS (SEQ ID NO: 15), GGGGSGGGGSGGGGS (SEQ ID NO: 16), or GGGASGGGGS (SEQ ID NO: 17), GGGGSGGGGSGGGGS (SEQ ID NO: 18), GGGGSGGGGSGGGGSGGGGS (SEQ ID NO: 19), GCGGS (SEQ ID NO: 20), GCGGSGGGGSGGGGS (SEQ ID NO: 21), GGGGSENLYFQGGGGS (SEQ ID NO: 47), and the like. In some embodiments, exemplary linkers can consist of amino acid sequences including, but not limited to, GGSG (SEQ ID NO: 5), GGSGG (SEQ ID NO: 6), GSGSG (SEQ ID NO: 7), GSGGG (SEQ ID NO: 8), GGGSG (SEQ ID NO: 9), GSSSG (SEQ ID NO: 10), SGGGGG (SEQ ID NO: 11), GCGASGGGGSGGGGS (SEQ ID NO: 12), GCGASGGGGSGGGGS (SEQ ID NO: 13), GGGGSGGGGS (SEQ ID NO: 14), GGGASGGGGSGGGGS (SEQ ID NO: 15), GGGGSGGGGSGGGGS (SEQ ID NO: 16), or GGGASGGGGS (SEQ ID NO: 17), GGGGSGGGGSGGGGS (SEQ ID NO: 18), GGGGSGGGGSGGGGSGGGGS (SEQ ID NO: 19), GCGGS (SEQ ID NO: 20), GCGGSGGGGSGGGGS (SEQ ID NO: 21), GGGGSENLYFQGGGGS (SEQ ID NO: 47), and the like. In an exemplary embodiment, the linker (e.g., the linker linking the MHC ligand peptide to the MHC class II molecule) comprises GCGGSGGGGSGGGGS (SEQ ID NO: 21). In an exemplary embodiment, the linker (e.g., the linker linking the MHC ligand peptide to the MHC class II molecule) consists essentially of GCGGSGGGGSGGGGS (SEQ ID NO: 21). In an exemplary embodiment, the linker (e.g., the linker linking the MHC ligand peptide to the MHC class II molecule) consists of GCGGSGGGGSGGGGS (SEQ ID NO: 21).

In some embodiments of linkers, a linker polypeptide includes a cysteine residue that can form a disulfide bond with a cysteine residue present in the MHC class II molecule. In an exemplary embodiment, the linker can include a cysteine residue that can form a disulfide bond with a cysteine residue present in the MHC class II α chain or portion or fragment or variant thereof or the MHC class II β chain or portion or fragment or variant thereof. As a non-limiting example, the cysteine can be the first, second, third, or fourth amino acid of the linker (e.g., but not limited to, the second amino acid of the linker).

Although the above linkers are described for linking the MHC class II molecule (e.g., but not limited to, an MHC class II β chain or portion or fragment or variant thereof or MHC class II α chain or portion or fragment or variant thereof) and the MHC ligand peptide, these linkers can also be used in any other context described herein where a linker is used.

(2) Disulfide Bridges

In some embodiments of complexes, the MHC ligand peptide or a linker connecting the MHC ligand peptide to the MHC class II molecule is attached via a disulfide bridge (i.e., a disulfide bond that extends between a pair of oxidized cysteines to at least one chain of the MHC class II molecule or a portion or fragment or variant thereof. In an exemplary embodiment, the MHC ligand peptide can comprise a first cysteine or the linker can comprise the first cysteine, and the MHC class II molecule can comprise a second cysteine in a proximal position to the first cysteine in the tertiary structure of the complex such that a disulfide bridge is formed and links the MHC ligand peptide in the peptide-binding groove of the MHC class II molecule. Tertiary structure refers to the three-dimensional structure resulting from folding and covalent cross-linking of the protein. Proximity can be determined, for example, based on available crystal structures. Such a disulfide bond can help position an MHC ligand peptide in the peptide-binding groove of the MHC class II molecule. Optionally, in some embodiments, the MHC ligand peptide can comprise a first cysteine and no other cysteines, or the linker can comprise the first cysteine and no other cysteines. Optionally, in some embodiments, if the MHC ligand peptide comprises the first cysteine, the linker does not comprise any other cysteines. Optionally, in some embodiments, if the linker comprises the first cysteine, the MHC ligand peptide does not comprise any other cysteines. Optionally, in some embodiments, the MHC class II molecule comprises only the second cysteine and no other cysteines or no other unpaired cysteines (for example, no Cys being able to form a disulfide bond within the MHC class II molecule). Optionally, in some embodiments, if the second cysteine is in the MHC class II α chain or portion or fragment or variant thereof, the MHC class II α chain or portion or fragment or variant thereof does not comprise any other cysteines or any other unpaired cysteines (for example, no Cys being able to form a disulfide bond within the MHC class II molecule). Optionally, in some embodiments, if the second cysteine is in the MHC class II β chain or portion or fragment or variant thereof, the MHC class II β chain or portion or fragment or variant thereof does not comprise any other cysteines or any other unpaired cysteines (for example, no Cys being able to form a disulfide bond within the MHC class II molecule). The cysteine in the MHC class II molecule can be a naturally occurring cysteine (i.e., present in an unmodified (i.e., wild type) MHC class II molecule) or it can be a mutation (addition or substitution) relative to the unmodified (i.e., wild type) the MHC class II molecule. Likewise, in some embodiments, the cysteine in the MHC ligand peptide can be a naturally occurring cysteine or it can be a mutation (addition or substitution) in the MHC ligand peptide. If it is a mutation in the MHC ligand peptide, preferably it is facing away from the epitope formed by the peptide-MHC class II complex. The chain (or portion or fragment or variant thereof) of the MHC class II molecule to which the linker is connected (i.e., covalently connected) and the chain (or portion or fragment or variant thereof) of the MHC class II molecule forming a disulfide bridge can be the same chain of the MHC class II molecule or different chains. In an exemplary embodiment, the linker can be connected to the β chain (i.e., the MHC class II β chain or a portion or fragment or variant thereof) of the MHC class II molecule, and the disulfide bridge can form between the cysteine in the MHC ligand peptide or linker and the α chain (i.e., the MHC class II α chain or a portion or fragment or variant thereof) of the MHC class II molecule. In some embodiments, the linker can be connected to the α chain (i.e., the MHC class II α chain or a portion or fragment or variant thereof) of the MHC class II molecule, and the disulfide bridge can form between the cysteine in the MHC ligand peptide or linker and the β chain (i.e., the MHC class II β chain or a portion or fragment or variant thereof) of the MHC class II molecule. In some embodiments, the linker can be connected to the α chain (i.e., the MHC class II α chain or a portion or fragment or variant thereof) of the MHC class II molecule, and the disulfide bridge can form between the cysteine in the MHC ligand peptide or linker and the α chain (i.e., the MHC class II α chain or a portion or fragment or variant thereof) of the MHC class II molecule. In some embodiments, the linker can be connected to the β chain (i.e., the MHC class II β chain or a portion or fragment or variant thereof) of the MHC class II molecule, and the disulfide bridge can form between the cysteine in the MHC ligand peptide or linker and the β chain (i.e., the MHC class II β chain or a portion or fragment or variant thereof) of the MHC class II molecule.

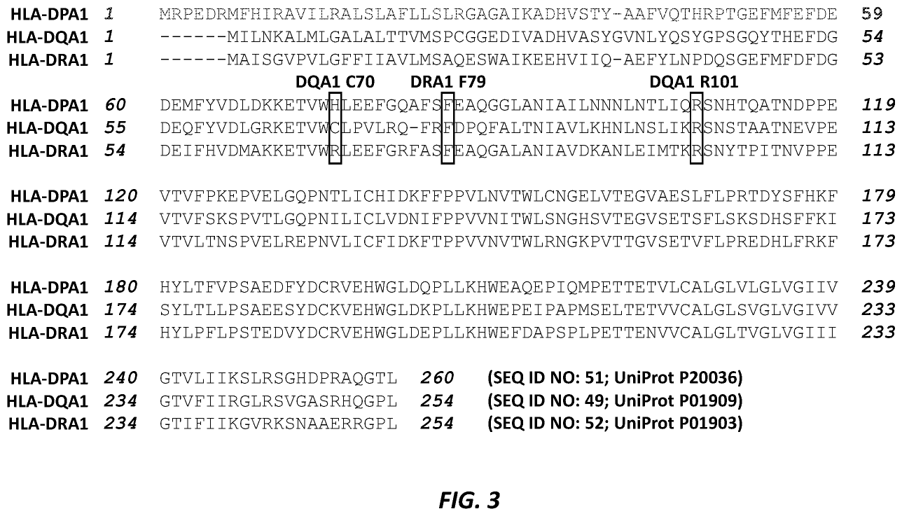

In some embodiments of peptide-MHC class II complexes, a residue that is not a cysteine in an unmodified (i.e., wild type) α chain (i.e., the MHC class II α chain or portion or fragment or variant thereof) of the MHC class II molecule or an unmodified (i.e., wild type) R chain (i.e., the MHC class II β chain or portion or fragment or variant thereof) of the MHC class II molecule can be mutated to cysteine based on proximity in the tertiary structure of the complex to the cysteine in the MHC ligand peptide or the linker connecting the MHC ligand peptide to the MHC class II molecule. In some embodiments, in some embodiments of peptide-MHC class II complexes, a cysteine residue can be inserted into the MHC class II α chain or portion or fragment or variant thereof or the MHC class II β chain or portion or fragment or variant thereof of the MHC class II molecule based on proximity in the tertiary structure of the complex to the cysteine in the MHC ligand peptide or the linker connecting the MHC ligand peptide to the MHC class II molecule. That is, the MHC class II molecule in the complex can be mutated relative to a corresponding wild type MHC class II molecule to a include a second cysteine in a position proximal to the first cysteine (i.e., in the MHC ligand peptide or the linker) in the tertiary structure of the complex. Proximity can be determined, for example, based on available crystal structures. In an exemplary embodiment, position 101 in the MHC class II α chain sequence set forth in SEQ ID NO: 49 or SEQ ID NO: 55 or 56 can be mutated to cysteine (R101C). Non-limiting examples of MHC class II α chain sequences with this position mutated to cysteine are set forth in SEQ ID NO: 53, SEQ ID NO: 54, and SEQ ID NO: 57. In some embodiments, a position in the sequence of the subject MHC class II α chain or portion or fragment or variant thereof corresponding to position 101 in the MHC class II α chain sequence set forth in SEQ ID NO: 49 when the sequence of the subject MHC class II α chain or portion or fragment or variant thereof is optimally aligned with SEQ ID NO: 49 can be mutated to cysteine (e.g., a position corresponding to the position labeled DQA1 R101 in the alignment of HLA-DPA1, HLA-DQA1, and HLA-DRA1 full-length sequences in FIG. 3 can be mutated to cysteine). For example, position 107 in SEQ ID NO: 51 or a position of the subject MHC class II α chain or portion or fragment or variant thereof corresponding to position 107 in SEQ ID NO: 51 when the sequence of the subject MHC class II α chain or portion or fragment or variant thereof is optimally aligned with SEQ ID NO: 51 can be mutated to cysteine. As another example, position 101 in SEQ ID NO: 52 or a position of the subject MHC class II α chain or portion or fragment or variant thereof corresponding to position 101 in SEQ ID NO: 52 when the sequence of the subject MHC class II α chain or portion or fragment or variant thereof is optimally aligned with SEQ ID NO: 52 can be mutated to cysteine. As yet another example, position 78 in SEQ ID NO: 59 or a position of the subject MHC class II α chain or portion or fragment or variant thereof corresponding to position 78 in SEQ ID NO: 59 when the sequence of the subject MHC class II α chain or portion or fragment or variant thereof is optimally aligned with SEQ ID NO: 59 can be mutated to cysteine. As yet another example, position 79 in SEQ ID NO: 61 or a position of the subject MHC class II α chain or portion or fragment or variant thereof corresponding to position 79 in SEQ ID NO: 61 when the sequence of the subject MHC class II α chain or portion or fragment or variant thereof is optimally aligned with SEQ ID NO: 61 can be mutated to cysteine. As yet another example, position 76 in SEQ ID NO: 62 or a position of the subject MHC class II α chain or portion or fragment or variant thereof corresponding to position 76 in SEQ ID NO: 62 when the sequence of the subject MHC class II α chain or portion or fragment or variant thereof is optimally aligned with SEQ ID NO: 62 can be mutated to cysteine. As another embodiment, position 79 in the MHC class II α chain sequence set forth in SEQ ID NO: 52 (HLA class II histocompatibility antigen, DR alpha chain; NCBI Accession No. P01903.1) can be mutated to cysteine (F79C). A non-limiting example of an MHC class II α chain sequence with this position mutated to cysteine is set forth in SEQ ID NO: 58. In some embodiments, a position in the sequence of the subject MHC class II α chain or portion or fragment or variant thereof corresponding to position 79 in the MHC class II α chain sequence set forth in SEQ ID NO: 52 when the sequence of the subject MHC class II α chain or portion or fragment or variant thereof is optimally aligned with SEQ ID NO: 52 can be mutated to cysteine (e.g., a position corresponding to the position labeled DRA1 F79 in the alignment of HLA-DPA1, HLA-DQA1, and HLA-DRA1 full-length sequences in FIG. 3 can be mutated to cysteine). For example, position 79 in SEQ ID NO: 49 or a position of the subject MHC class II α chain or portion or fragment or variant thereof corresponding to position 79 in SEQ ID NO: 49 when the sequence of the subject MHC class II α chain or portion or fragment or variant thereof is optimally aligned with SEQ ID NO: 49 can be mutated to cysteine. As another example, position 85 in SEQ ID NO: 51 or a position of the subject MHC class II α chain or portion or fragment or variant thereof corresponding to position 85 in SEQ ID NO: 51 when the sequence of the subject MHC class II α chain or portion or fragment or variant thereof is optimally aligned with SEQ ID NO: 51 can be mutated to cysteine. As yet another example, position 56 in SEQ ID NO: 59 or a position of the subject MHC class II α chain or portion or fragment or variant thereof corresponding to position 56 in SEQ ID NO: 59 when the sequence of the subject MHC class II α chain or portion or fragment or variant thereof is optimally aligned with SEQ ID NO: 59 can be mutated to cysteine. As yet another example, position 57 in SEQ ID NO: 61 or a position of the subject MHC class II α chain or portion or fragment or variant thereof corresponding to position 57 in SEQ ID NO: 61 when the sequence of the subject MHC class II α chain or portion or fragment or variant thereof is optimally aligned with SEQ ID NO: 61 can be mutated to cysteine. As yet another example, position 54 in SEQ ID NO: 62 or a position of the subject MHC class II α chain or portion or fragment or variant thereof corresponding to position 54 in SEQ ID NO: 62 when the sequence of the subject MHC class II α chain or portion or fragment or variant thereof is optimally aligned with SEQ ID NO: 62 can be mutated to cysteine.

In some embodiments of peptide-MHC class II complexes, a residue that is a cysteine in an unmodified (i.e., wild type) α chain (i.e., the MHC class II α chain or portion or fragment or variant thereof) of the MHC class II molecule or an unmodified (i.e., wild type) β chain (i.e., the MHC class II β chain or portion or fragment or variant thereof) of the MHC class II molecule can be mutated to a non-cysteine residue to minimize disulfide scrambling (i.e., disulfide bonds forming between cysteine residues other than those intended to be used). The amino acid that is substituted for the cysteine can be chosen to allow proper folding of the MHC complex. This can be determined, for example, based on available crystal structures or by inspection of sequence alignments of closely related MHC sequences. In one exemplary embodiment, the cysteine is mutated to alanine because it has a minimal side chain and is therefore least disruptive sterically. In one exemplary embodiment, the cysteine at position 70 in the MHC class II α chain sequence set forth in SEQ ID NO: 49 (HLA class II histocompatibility antigen, DQ alpha 1 chain; NCBI Accession No. P01909.1) can be mutated. As a non-limiting example, it can be mutated to Ala, Trp, Arg, or Gln (unpaired Cys in DQA1*0501 is substituted by Trp, Arg, or Gln in closest MHC sequences from other species based on sequence alignments). In an exemplary embodiment, the cysteine at position 70 in the MHC class II α chain sequence set forth in SEQ ID NO: 49 or 53 can be mutated to alanine (C70A). Non-limiting examples of MHC class II α chain sequences with this position mutated to alanine are set forth in SEQ ID NOS: 56 and 54. In an exemplary embodiment, the cysteine at position 70 in the MHC class II α chain sequence set forth in SEQ ID NO: 49 can be mutated to glutamine (C70Q). Non-limiting examples of MHC class II α chain sequences with this position mutated to glutamine are set forth in SEQ ID NOS: 55 and 57. In some embodiments, a cysteine in the sequence of the subject MHC class II α chain sequence or portion or fragment or variant thereof corresponding to position 70 in the MHC class II α chain sequence set forth in SEQ ID NO: 49 when the sequence of the subject MHC class II α chain or portion or fragment or variant thereof is optimally aligned with SEQ ID NO: 49 can be mutated (e.g., but not limited to, to alanine or glutamine) (e.g., a position corresponding to the position labeled DQA1 C70 in the alignment of HLA-DPA1, HLA-DQA1, and HLA-DRA1 full-length sequences in FIG. 3 can be mutated). For example, a position of the subject MHC class II α chain or portion or fragment or variant thereof corresponding to position 75 in SEQ ID NO: 51 when the sequence of the subject MHC class II α chain or portion or fragment or variant thereof is optimally aligned with SEQ ID NO: 51 can be mutated. As another example, a position of the subject MHC class II α chain or portion or fragment or variant thereof corresponding to position 69 in SEQ ID NO: 52 when the sequence of the subject MHC class II α chain or portion or fragment or variant thereof is optimally aligned with SEQ ID NO: 52 can be mutated. As yet another example, the cysteine at position 47 in the MHC class II α chain sequence set forth in SEQ ID NO: 59 or a position of the subject MHC class II α chain or portion or fragment or variant thereof corresponding to position 47 in SEQ ID NO: 59 when the sequence of the subject MHC class II α chain or portion or fragment or variant thereof is optimally aligned with SEQ ID NO: 59 can be mutated. As yet another example, a position of the subject MHC class II α chain or portion or fragment or variant thereof corresponding to position 47 in SEQ ID NO: 61 when the sequence of the subject MHC class II α chain or portion or fragment or variant thereof is optimally aligned with SEQ ID NO: 61 can be mutated. As yet another example, a position of the subject MHC class II α chain or portion or fragment or variant thereof corresponding to position 44 in SEQ ID NO: 62 when the sequence of the subject MHC class II α chain or portion or fragment or variant thereof is optimally aligned with SEQ ID NO: 62 can be mutated.

In one exemplary embodiment, the linker connecting the MHC ligand peptide to the MHC class II molecule can comprise the first cysteine, and the MHC class II molecule can comprise a second cysteine in a proximal position such that a disulfide bridge is formed and links the MHC ligand peptide in the peptide-binding groove of the MHC class II molecule. Optionally, in some embodiments, position 101 in the MHC class II α chain sequence set forth in SEQ ID NO: 49 can be mutated to cysteine (R101C) or a position in the sequence of the subject MHC class II α chain or portion or fragment or variant thereof corresponding to position 101 in the MHC class II α chain sequence set forth in SEQ ID NO: 49 when the sequence of the subject MHC class II α chain or portion or fragment or variant thereof is optimally aligned with SEQ ID NO: 49 can be mutated to cysteine. Optionally, in some embodiments, the cysteine at position 70 in the MHC class II α chain sequence set forth in SEQ ID NO: 49 can be mutated (e.g., but not limited to, to Ala, Trp, Arg, or Gln) or a cysteine in the sequence of the subject MHC class II α chain or portion or fragment or variant thereof corresponding to position 70 in the MHC class II α chain sequence set forth in SEQ ID NO: 49 when the sequence of the subject MHC class II α chain or portion or fragment or variant thereof is optimally aligned with SEQ ID NO: 49 can be mutated (e.g., but not limited to, to alanine or glutamine). Optionally, in some embodiments, the linker comprises a cysteine in the first 3 or 4 residues, such as the first residue, the second residue, the third residue, or the fourth residue (and optionally, in some embodiments, does not comprise any additional cysteines). In an exemplary embodiment, the linker can comprise a cysteine at position 3. In another exemplary embodiment, the linker can comprise a cysteine at position 2. Optionally, in some embodiments, the linker comprises 15 amino acids (such as GCGGSGGGGSGGGGS (SEQ ID NO: 21)), including a Cys for disulfide linkage to the MHC class II α chain position 101 (or a position in the sequence of the subject MHC class II α chain or portion or fragment or variant thereof corresponding to position 101 in the MHC class II α chain sequence set forth in SEQ ID NO: 49 when the sequence of the subject MHC class II α chain or portion or fragment or variant thereof is optimally aligned with SEQ ID NO: 49). Optionally, in some embodiments, the linker consists essentially of 15 amino acids (such as GCGGSGGGGSGGGGS (SEQ ID NO: 21)), including a Cys for disulfide linkage to the MHC class II α chain position 101 (or a position in the sequence of the subject MHC class II α chain or portion or fragment or variant thereof corresponding to position 101 in the MHC class II α chain sequence set forth in SEQ ID NO: 49 when the sequence of the subject MHC class II α chain or portion or fragment or variant thereof is optimally aligned with SEQ ID NO: 49). Optionally, in some embodiments, the linker consists of 15 amino acids (such as GCGGSGGGGSGGGGS (SEQ ID NO: 21)), including a Cys for disulfide linkage to the MHC class II α chain position 101 (or a position in the sequence of the subject MHC class II α chain or portion or fragment or variant thereof corresponding to position 101 in the MHC class II α chain sequence set forth in SEQ ID NO: 49 when the sequence of the subject MHC class II α chain or portion or fragment or variant thereof is optimally aligned with SEQ ID NO: 49). Optionally, in some embodiments, the MHC class II molecule is an HLA-DQ MHC class II molecule (e.g., but not limited to, HLA-DQ2), an HLA-DR MHC class II molecule (e.g., but not limited to, HLA-DR2), or an HLA-DP MHC class II molecule.

In another embodiment, the MHC ligand peptide can comprise the first cysteine, and the MHC class II molecule can comprise a second cysteine in a proximal position such that a disulfide bridge is formed and links the MHC ligand peptide in the peptide-binding groove of the MHC class II molecule. In one exemplary embodiment, the P1 anchor position in the MHC ligand peptide can be a cysteine. In another embodiment, the P4 anchor position in the MHC ligand peptide can be a cysteine. In another embodiment, the P6 anchor position in the MHC ligand peptide can be a cysteine. In another embodiment, the P9 anchor position in the MHC ligand peptide can be a cysteine. Optionally, in some embodiments, the cysteine at position 70 in the MHC class II α chain sequence set forth in SEQ ID NO: 49 can be mutated (e.g., but not limited to, to Ala, Trp, Arg, or Gln) or a cysteine in the sequence of the subject MHC class II α chain or portion or fragment or variant thereof corresponding to position 70 in the MHC class II α chain sequence set forth in SEQ ID NO: 49 when the sequence of the subject MHC class II α chain or portion or fragment or variant thereof is optimally aligned with SEQ ID NO: 49 can be mutated (e.g., but not limited to, to alanine or glutamine). Optionally, in some embodiments, the MHC ligand peptide comprises a cysteine in the first 3 or 4 residues, such as the first residue, the second residue, the third residue, or the fourth residue (and optionally, in some embodiments, does not comprise any additional cysteines). Optionally, in some embodiments, the linker comprises 15 amino acids (such as GCGGSGGGGSGGGGS (SEQ ID NO: 21)), including a Cys for disulfide linkage to the MHC class II α chain position 101 (or a position in the sequence of the subject MHC class II α chain or portion or fragment or variant thereof corresponding to position 101 in the MHC class II α chain sequence set forth in SEQ ID NO: 49 when the sequence of the subject MHC class II α chain or portion or fragment or variant thereof is optimally aligned with SEQ ID NO: 49). Optionally, in some embodiments, the linker consists essentially of 15 amino acids (such as GCGGSGGGGSGGGGS (SEQ ID NO: 21)), including a Cys for disulfide linkage to the MHC class II α chain position 101 (or a position in the sequence of the subject MHC class II α chain or portion or fragment or variant thereof corresponding to position 101 in the MHC class II α chain sequence set forth in SEQ ID NO: 49 when the sequence of the subject MHC class II α chain or portion or fragment or variant thereof is optimally aligned with SEQ ID NO: 49). Optionally, in some embodiments, the linker consists of 15 amino acids (such as GCGGSGGGGSGGGGS (SEQ ID NO: 21)), including a Cys for disulfide linkage to the MHC class II α chain position 101 (or a position in the sequence of the subject MHC class II α chain or portion or fragment or variant thereof corresponding to position 101 in the MHC class II α chain sequence set forth in SEQ ID NO: 49 when the sequence of the subject MHC class II α chain or portion or fragment or variant thereof is optimally aligned with SEQ ID NO: 49). Optionally, in some embodiments, the MHC class II molecule is an HLA-DQ MHC class II molecule (e.g., but not limited to, HLA-DQ2), an HLA-DR MHC class II molecule (e.g., but not limited to, HLA-DR2), or an HLA-DP MHC class II molecule.

C. Other Components

In some embodiments of the present invention, the compositions comprising peptide-MHC class II complexes can also comprise other components. As non-limiting examples, the compositions may also comprise one or more peptides or one or more other molecules capable of stimulating T helper cells or may also comprise one or more immunostimulatory molecules capable of boosting an immune response. Such T helper cell epitopes or immunostimulatory molecules may be linked (e.g., but not limited to, covalently linked) to the peptide-MHC class II complex, or they can be admixed in a composition with but not physically linked to the peptide-MHC class II complex. In an exemplary embodiment, such T helper cell epitopes or immunostimulatory molecules may be linked (e.g., but not limited to, covalently linked) to the peptide-MHC class II complex (e.g., but not limited to, to the C-terminus of the peptide-MHC class II complex). As non-limiting examples, the T helper cell epitopes or immunostimulatory molecules may be indirectly or directly linked to the C-terminus of the MHC class II molecule (e.g., but not limited to, the MHC class II α chain or portion or fragment or variant thereof and/or the MHC class II β chain or portion or fragment or variant thereof). Covalent linkage can be direct or via a linker such as a peptide linker. In some embodiments, non-limiting examples of linkers suitable for use in the disclosed peptide-MHC class II complexes are described elsewhere herein.

As a non-limiting example of some embodiments, a T helper cell epitope suitable for use in the disclosed peptide-MHC class II complexes is pan-DR-binding epitope (PADRE) peptides or molecules that are designed on the basis of their binding activity to most HLA-DR (human MHC class II) molecules. PADRE is a “pan-DR-binding epitope” that is a mouse MHC-II binding sequence used to boost the immune response based on providing a “universal” MHC-II epitope that is presented by mouse MHC I-A^{b} haplotype as described in Alexander et al. (2000) J. Immunol. 164(3):1625-1633, herein incorporated by reference in its entirety for all purposes. It can be fused the termini of antigen used in the immunization, and its uptake by antigen-presenting cells and presentation by MHC-II improves overall immune response. See, e.g., U.S. Pat. Nos. 6,413,935; 5,736,142; and Alexander et al. (1994)Immunity 1(9):751-761, each of which is incorporated herein by reference in its entirety for all purposes. These peptides have been shown to help in the generation of various immune responses against antigens. In some embodiments, the PADRE peptide can comprise AKFVAAWTLKAAA (SEQ ID NO: 25). In some embodiments, the PADRE peptide can consist essentially of AKFVAAWTLKAAA (SEQ ID NO: 25). In some embodiments, the PADRE peptide can consist of AKFVAAWTLKAAA (SEQ ID NO: 25).

Like PADRE, peptides from lymphocytic choriomeningitis virus (LCMV) (e.g., but not limited to, from LCMV glycoprotein (GP), nucleoprotein (NP), or zinc-binding protein (Z)) are an alternate MHC-II binding small polypeptide that can be used to boost immune response. In some embodiments, such peptides can be used in addition to or as an alternative to PADRE. In some embodiments, the LCMV peptide used can be an LCMV-specific MHC class II-restricted CD4+ T cell epitope. In some embodiments, the LCMV peptide used can comprise one or more of the following sequences: TMFEALPHIIDEVIN (epitope GP₆₋₂₀; SEQ ID NO: 26); GIKAVYNFATCGIFA (epitope GP₃₁₋₄₅; SEQ ID NO: 27); DIYKGVYQFKSVEFD (epitope GP₆₆₋₈₀; SEQ ID NO: 28); TSAFNKKTFDHTLMS (epitope GP₁₂₆₋₁₄₀; SEQ ID NO: 29); DAQSAQSQCRTFRGR (epitope GP₁₇₆₋₁₉₀; SEQ ID NO: 30); TFRGRVLDMFRTAFG (epitope GP₁₈₆₋₂₀₀; SEQ ID NO: 31); CDMLRLIDYNKAALS (epitope GP₃₁₆₋₃₃₀; SEQ ID NO: 32); IEQEADNMITEMLRK (epitope GP₄₀₉₋₄₂₃; SEQ ID NO: 33); EVKSFQWTQALRREL (epitope NP₆₋₂₀; SEQ ID NO: 34); KNVLKVGRLSAEELM (epitope NP₈₆₋₁₀₀; SEQ ID NO: 35); SERPQASGVYMGNLT (epitope NP₁₁₆₋₁₃₀; SEQ ID NO: 36); PSLTMACMAKQSQTP (epitope NP₁₇₆₋₁₉₀; SEQ ID NO: 37); EGWPYIACRTSIVGR (epitope NP₃₁₁₋₃₂₅; (SEQ ID NO: 38); SQNRKDIKLIDVEMT (epitope NP₄₆₆₋₄₈₀; SEQ ID NO: 39); GWLCKMHTGIVRDKK (epitope NP₄₉₆₋₅₁₀; SEQ ID NO: 40); and SCKSCWQKFDSLVRC (epitope Z₃₁₋₄₅; SEQ ID NO: 41). In some embodiments, the LCMV peptide used can consist essentially of one or more of the following sequences: TMFEALPHIIDEVIN (epitope GP₆₋₂₀; SEQ ID NO: 26); GIKAVYNFATCGIFA (epitope GP₃₁₋₄₅; SEQ ID NO: 27); DIYKGVYQFKSVEFD (epitope GP₆₆₋₈₀; SEQ ID NO: 28); TSAFNKKTFDHTLMS (epitope GP₁₂₆₋₁₄₀; SEQ ID NO: 29); DAQSAQSQCRTFRGR (epitope GP₁₇₆₋₁₉₀; SEQ ID NO: 30); TFRGRVLDMFRTAFG (epitope GP₁₈₆₋₂₀₀; SEQ ID NO: 31); CDMLRLIDYNKAALS (epitope GP₃₁₆₋₃₃₀; SEQ ID NO: 32); IEQEADNMITEMLRK (epitope GP₄₀₉₋₄₂₃; SEQ ID NO: 33); EVKSFQWTQALRREL (epitope NP₆₋₂₀; SEQ ID NO: 34); KNVLKVGRLSAEELM (epitope NP₈₆₋₁₀₀; SEQ ID NO: 35); SERPQASGVYMGNLT (epitope NP₁₁₆₋₁₃₀; SEQ ID NO: 36); PSLTMACMAKQSQTP (epitope NP₁₇₆₋₁₉₀; SEQ ID NO: 37); EGWPYIACRTSIVGR (epitope NP₃₁₁₋₃₂₅; (SEQ ID NO: 38); SQNRKDIKLIDVEMT (epitope NP₄₆₆₋₄₈₀; SEQ ID NO: 39); GWLCKMHTGIVRDKK (epitope NP₄₉₆₋₅₁₀; SEQ ID NO: 40); and SCKSCWQKFDSLVRC (epitope Z₃₁₋₄₅; SEQ ID NO: 41). In some embodiments, the LCMV peptide used can consist of one or more of the following sequences: TMFEALPHIIDEVIN (epitope GP₆₋₂₀; SEQ ID NO: 26); GIKAVYNFATCGIFA (epitope GP₃₁₋₄₅; SEQ ID NO: 27); DIYKGVYQFKSVEFD (epitope GP₆₆₋₈₀; SEQ ID NO: 28); TSAFNKKTFDHTLMS (epitope GP₁₂₆₋₁₄₀; SEQ ID NO: 29); DAQSAQSQCRTFRGR (epitope GP₁₇₆₋₁₉₀; SEQ ID NO: 30); TFRGRVLDMFRTAFG (epitope GP₁₈₆₋₂₀₀; SEQ ID NO: 31); CDMLRLIDYNKAALS (epitope GP₃₁₆₋₃₃₀; SEQ ID NO: 32); IEQEADNMITEMLRK (epitope GP₄₀₉₋₄₂₃; SEQ ID NO: 33); EVKSFQWTQALRREL (epitope NP₆₋₂₀; SEQ ID NO: 34); KNVLKVGRLSAEELM (epitope NP₈₆₋₁₀₀; SEQ ID NO: 35); SERPQASGVYMGNLT (epitope NP₁₁₆₋₁₃₀; SEQ ID NO: 36); PSLTMACMAKQSQTP (epitope NP₁₇₆₋₁₉₀; SEQ ID NO: 37); EGWPYIACRTSIVGR (epitope NP₃₁₁₋₃₂₅; (SEQ ID NO: 38); SQNRKDIKLIDVEMT (epitope NP₄₆₆₋₄₈₀; SEQ ID NO: 39); GWLCKMHTGIVRDKK (epitope NP₄₉₆₋₅₁₀; SEQ ID NO: 40); and SCKSCWQKFDSLVRC (epitope Z₃₁₋₄₅; SEQ ID NO: 41). See, e.g., Botten et al. (2010) Microbiol. Mol. Biol. Rev. 74(2):157-170 and Mothe et al. (2007) J. Immunol. 179(2):1058-1067, each of which is herein incorporated by reference in its entirety for all purposes. In some embodiments, the peptide can comprise SERPQASGVYMGNLT (SEQ ID NO: 36). In some embodiments, the peptide can consist essentially of SERPQASGVYMGNLT (SEQ ID NO: 36). In some embodiments, the peptide can consist of SERPQASGVYMGNLT (SEQ ID NO: 36).

In some embodiments, one T cell epitope (e.g., LCMV peptide) is added to the compositions comprising peptide-MHC class II complexes. In some embodiments, a plurality of T cell epitopes (e.g., but not limited to, 2 T cell epitopes, 3 T cell epitopes, 4 T cell epitopes, 5 T cell epitopes, 6 T cell epitopes, 7 T cell epitopes, 8 T cell epitopes, 9 T cell epitopes, or 10 T cell epitopes) is added to the compositions comprising peptide-MHC class II complexes. In some embodiments, about 1 to about 10 T cell epitopes can be added to the compositions comprising peptide-MHC class II complexes. In some embodiments, about 2 to about 10 T cell epitopes can be added to the compositions comprising peptide-MHC class II complexes. In some embodiments, about 3 to about 10 T cell epitopes can be added to the compositions comprising peptide-MHC class II complexes. In some embodiments, about 4 to about 10 T cell epitopes can be added to the compositions comprising peptide-MHC class II complexes. In some embodiments, about 5 to about 10 T cell epitopes can be added to the compositions comprising peptide-MHC class II complexes. In some embodiments, about 6 to about 10 T cell epitopes can be added to the compositions comprising peptide-MHC class II complexes. In some embodiments, about 7 to about 10 T cell epitopes can be added to the compositions comprising peptide-MHC class II complexes. In some embodiments, about 8 to about 10 T cell epitopes can be added to the compositions comprising peptide-MHC class II complexes. In some embodiments, about 9 to about 10 T cell epitopes can be added to the compositions comprising peptide-MHC class II complexes. In some embodiments, about 1 to about 9 T cell epitopes can be added to the compositions comprising peptide-MHC class II complexes. In some embodiments, about 1 to about 8 T cell epitopes can be added to the compositions comprising peptide-MHC class II complexes. In some embodiments, about 1 to about 7 T cell epitopes can be added to the compositions comprising peptide-MHC class II complexes. In some embodiments, about 1 to about 6 T cell epitopes can be added to the compositions comprising peptide-MHC class II complexes. In some embodiments, about 1 to about 5 T cell epitopes can be added to the compositions comprising peptide-MHC class II complexes. In some embodiments, about 1 to about 4 T cell epitopes can be added to the compositions comprising peptide-MHC class II complexes. In some embodiments, about 1 to about 3 T cell epitopes can be added to the compositions comprising peptide-MHC class II complexes. In some embodiments, about 1 to about 2 T cell epitopes can be added to the compositions comprising peptide-MHC class II complexes. In some embodiments, about 2 to about 9 T cell epitopes can be added to the compositions comprising peptide-MHC class II complexes. In some embodiments, about 3 to about 8 T cell epitopes can be added to the compositions comprising peptide-MHC class II complexes. In some embodiments, about 4 to about 7 T cell epitopes can be added to the compositions comprising peptide-MHC class II complexes. In some embodiments, about 4 to about 6 T cell epitopes can be added to the compositions comprising peptide-MHC class II complexes. In some embodiments, about 2 to about 4 T cell epitopes can be added to the compositions comprising peptide-MHC class II complexes. In some embodiments, each of the plurality of T cell epitopes can be, but is not limited to, an LCMV peptide, where each LCMV peptide can comprise any of the sequences listed above. In some embodiments, each of the plurality of T cell epitopes can be, but is not limited to, an LCMV peptide, where each LCMV peptide can consist essentially of any of the sequences listed above. In some embodiments, each of the plurality of T cell epitopes can be, but is not limited to, an LCMV peptide, where each LCMV peptide can consist of any of the sequences listed above.

In some embodiments, one T cell epitope (e.g., LCMV peptide) is added to the compositions comprising peptide-MHC class II complexes. In some embodiments, a plurality of T cell epitopes (e.g., but not limited to, 2 T cell epitopes, 3 T cell epitopes, 4 T cell epitopes, 5 T cell epitopes, 6 T cell epitopes, 7 T cell epitopes, 8 T cell epitopes, 9 T cell epitopes, or 10 T cell epitopes) is added to the compositions comprising peptide-MHC class II complexes. In some embodiments, 1 to 10 T cell epitopes can be added to the compositions comprising peptide-MHC class II complexes. In some embodiments, 2 to 10 T cell epitopes can be added to the compositions comprising peptide-MHC class II complexes. In some embodiments, 3 to 10 T cell epitopes can be added to the compositions comprising peptide-MHC class II complexes. In some embodiments, 4 to 10 T cell epitopes can be added to the compositions comprising peptide-MHC class II complexes. In some embodiments, 5 to 10 T cell epitopes can be added to the compositions comprising peptide-MHC class II complexes. In some embodiments, 6 to 10 T cell epitopes can be added to the compositions comprising peptide-MHC class II complexes. In some embodiments, 7 to 10 T cell epitopes can be added to the compositions comprising peptide-MHC class II complexes. In some embodiments, 8 to 10 T cell epitopes can be added to the compositions comprising peptide-MHC class II complexes. In some embodiments, 9 to 10 T cell epitopes can be added to the compositions comprising peptide-MHC class II complexes. In some embodiments, 1 to 9 T cell epitopes can be added to the compositions comprising peptide-MHC class II complexes. In some embodiments, 1 to 8 T cell epitopes can be added to the compositions comprising peptide-MHC class II complexes. In some embodiments, 1 to 7 T cell epitopes can be added to the compositions comprising peptide-MHC class II complexes. In some embodiments, 1 to 6 T cell epitopes can be added to the compositions comprising peptide-MHC class II complexes. In some embodiments, 1 to 5 T cell epitopes can be added to the compositions comprising peptide-MHC class II complexes. In some embodiments, 1 to 4 T cell epitopes can be added to the compositions comprising peptide-MHC class II complexes. In some embodiments, 1 to 3 T cell epitopes can be added to the compositions comprising peptide-MHC class II complexes. In some embodiments, 1 to 2 T cell epitopes can be added to the compositions comprising peptide-MHC class II complexes. In some embodiments, 2 to 9 T cell epitopes can be added to the compositions comprising peptide-MHC class II complexes. In some embodiments, 3 to 8 T cell epitopes can be added to the compositions comprising peptide-MHC class II complexes. In some embodiments, 4 to 7 T cell epitopes can be added to the compositions comprising peptide-MHC class II complexes. In some embodiments, 4 to 6 T cell epitopes can be added to the compositions comprising peptide-MHC class II complexes. In some embodiments, 2 to 4 T cell epitopes can be added to the compositions comprising peptide-MHC class II complexes. In some embodiments, each of the plurality of T cell epitopes can be, but is not limited to, an LCMV peptide, where each LCMV peptide can comprise any of the sequences listed above. In some embodiments, each of the plurality of T cell epitopes can be, but is not limited to, an LCMV peptide, where each LCMV peptide can consist essentially of any of the sequences listed above. In some embodiments, each of the plurality of T cell epitopes can be, but is not limited to, an LCMV peptide, where each LCMV peptide can consist of any of the sequences listed above.

In some embodiments, other peptides or molecules can also be used in the compositions provided herein to improve immune response. Non-limiting examples include immunostimulatory agents such as keyhole limpet hemocyanin (KLH) or polypeptides that are cross-presented by multiple haplotypes of the immunization host (e.g., but not limited to, mouse or rat). Such peptides are molecules can be, for example, fused to the peptide-MHC class II complex or provided separately (e.g., but not limited to, admixed in the same composition).

In some embodiments, peptides or other tags can also be used in the compositions to facilitate, for example, purification. In some embodiments, non-limiting examples of tags suitable for use in the disclosed peptide-MHC class II complexes include, but are not limited to, E. coli biotin ligase (BirA), myc-myc-histidine (mmH), glutathione-s-transferase (GST), maltose binding protein (MBP), chitin binding protein (CBP), FLAG, and 1D4 (i.e., the 9 amino acid 1D4 epitope derived from the C-terminus of bovine rhodopsin). In some embodiments, the sequence of the BirA tag can comprise GLNDIFEAQKIEWHE (SEQ ID NO: 42). In some embodiments, the sequence of the BirA tag can consist essentially of GLNDIFEAQKIEWHE (SEQ ID NO: 42). In some embodiments, the sequence of the BirA tag can consist of GLNDIFEAQKIEWHE (SEQ ID NO: 42). In some embodiments, the sequence of the mmH tag can comprise EQKLISEEDLEQKLISEEDLHHHHHH (SEQ ID NO: 43) or EQKLISEEDLGGEQKLISEEDLHHHH-IHH (SEQ ID NO: 48). In some embodiments, the sequence of the mmH tag can consist essentially of EQKLISEEDLEQKLISEEDLHHHHHH (SEQ ID NO: 43) or EQKLISEEDLGGEQKLISEEDLH*IFHIH (SEQ ID NO: 48). In some embodiments, the sequence of the mmH tag can consist of EQKLISEEDLEQKLISEEDLHHI*FIHH (SEQ ID NO: 43) or EQKLISEEDLGGEQKLISEEDLHHHH-IHH (SEQ ID NO: 48).

### III. Nucleic Acids Encoding Peptide-MHC Class II Complexes

Also provided are nucleic acids encoding the peptide-MHC class II complexes disclosed in some embodiments of the invention herein. Such nucleic acids can be deoxyribonucleic acid (DNA), ribonucleic acid (RNA), or hybrids or derivatives of either DNA or RNA. Optionally, in some embodiments, the nucleic acid encoding the peptide-MHC class II complex can be codon-optimized for efficient translation into protein in a particular cell or organism. As a non-limiting example, the nucleic acid encoding the peptide-MHC class II complex can be modified to substitute codons having a higher frequency of usage in a human cell, a mammalian cell, a rodent cell, a mouse cell, a rat cell, or any other host cell of interest, as compared to the naturally occurring polynucleotide sequence. Any portion or fragment of a nucleic acid molecule can be produced by: (1) isolating the molecule from its natural milieu; (2) using recombinant DNA technology (e.g., but not limited to, PCR amplification or cloning); or (3) using chemical synthesis methods. Nucleic acids encoding the peptide-MHC class II complex can comprise modifications for improved stability or reduced immunogenicity. Non-limiting examples of modifications include: (1) alteration or replacement of one or both of the non-linking phosphate oxygens and/or of one or more of the linking phosphate oxygens in the phosphodiester backbone linkage; (2) alteration or replacement of a constituent of a ribose sugar such as alteration or replacement of the 2′ hydroxyl on the ribose sugar; (3) replacement of the phosphate moiety with dephospho linkers; (4) modification or replacement of a naturally occurring nucleobase; (5) replacement or modification of a ribose-phosphate backbone; (6) modification of the 3′ end or 5′ end of the oligonucleotide (e.g., but not limited to, removal, modification or replacement of a terminal phosphate group or conjugation of a moiety); and (7) modification of the sugar.

In some embodiments, the nucleic acids can be in the form of an expression construct as defined elsewhere herein. As a non-limiting example, the nucleic acids can include regulatory regions that control expression of the nucleic acid molecule (e.g., but not limited to, transcription or translation control regions), full-length or partial coding regions, and combinations thereof. As a non-limiting example, the nucleic acids can be operably linked to a promoter active in a cell or organism of interest. Promoters that can be used in such expression constructs include promoters active, for example, in one or more of a eukaryotic cell, such as a mammalian cell (e.g., a non-human mammalian cell or a human cell), such as a rodent cell (e.g., but not limited to, a mouse cell, or a rat cell). Such promoters can be, for example, conditional promoters, inducible promoters, constitutive promoters, or tissue-specific promoters.

In some embodiments, the nucleic acids can include functional equivalents of natural nucleic acid molecules encoding an MHC molecule or a peptide, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode a protein capable of forming the compositions comprising the peptide-MHC class II complexes described elsewhere herein (e.g., that can be recognized by T cell receptors).

### IV. Methods of Using Peptide-MHC Class H Complexes

Also provided are methods of eliciting an immune response in a subject, comprising administering to the subject an effective amount of a composition comprising a peptide-MHC class II complex as described elsewhere herein.

In some embodiments of the invention, a subject can include, for example, any type of animal or mammal. Mammals include, for example, humans, non-human mammals, non-human primates, monkeys, apes, cats, dogs, horses, bulls, deer, bison, sheep, rabbits, rodents (e.g., but not limited to, mice, rats, hamsters, and guinea pigs), and livestock (e.g., but not limited to, bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human mammal” excludes humans. Particular non-limiting examples of non-human mammals include rodents, such as mice and rats.

The term administering refers to administration of a composition to a subject or system (e.g., but not limited to, to a cell, organ, tissue, organism, or relevant component or set of components thereof). The route of administration may vary depending, for example, on the subject or system to which the composition is being administered, the nature of the composition, the purpose of the administration, and so forth. The term “administration” or “administering” is intended to include routes of introducing the peptide-MHC class II complexes to a subject to perform their intended function (e.g., but not limited to, induce or modulate an immune response). In some embodiments, non-limiting examples of routes of administration which can be used include injection (subcutaneous, intravenous, parenterally, intraperitoneally, intrathecal), oral, inhalation, rectal and transdermal. As non-limiting examples, administration to a subject (e.g., but not limited to, to a human or a rodent) may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. Peptide-MHC class II complexes can be administered in tablets or capsule form (e.g., but not limited to, by injection, inhalation, eye lotion, ointment, suppository, and so forth), topically by lotion or ointment, or rectally by suppositories. Administration can be in a bolus or can be by continuous infusion. Administration may involve intermittent dosing or continuous dosing (e.g., but not limited to, perfusion) for at least a selected period of time. Depending on the route of administration, the peptide-MHC class II complex can be coated with or disposed in a selected material to protect it from natural conditions which may detrimentally affect its ability to perform its intended function. The peptide-MHC class II complex can be administered alone, or in conjunction with either another agent (e.g., but not limited to, an immunostimulatory agent) or with a pharmaceutically-acceptable carrier, or both. The peptide-MHC class II complex can be administered prior to the administration of the other agent, simultaneously with the agent, or after the administration of the agent. Furthermore, the peptide-MHC class II complex can also be administered in a proform which is converted into its active metabolite, or more active metabolite in vivo.

Also provided are methods of making an antigen-binding protein, comprising immunizing a non-human animal with a peptide-MHC class II complex as described elsewhere herein, allowing the non-human animal to mount an immune response to the peptide MHC class II complex, and isolating a cell (e.g., but not limited to, a lymphocyte) or nucleic acid from the non-human animal, wherein the cell or nucleic acid comprises or encodes an antigen-binding protein that specifically binds the peptide-MHC-class II complex. As a non-limiting example, the antigen-binding domain can specifically bind (e.g., but not limited to, with an equilibrium dissociation constant (KD) in the micromolar, nanomolar, or picomolar range) an epitope of the peptide-MHC class II complex. In some embodiments, the antigen-binding protein can be a therapeutic antigen-binding protein or antibody (e.g. for use in a patient).

In one exemplary embodiment, the cell is a B cell and is isolated from the non-human animal, and the method further comprises identifying the immunoglobulin heavy and light chain variable region nucleic acid sequences that encode the immunoglobulin heavy and light chain variable domains that, when paired, specifically bind the peptide-MHC class II complex. Such methods can further comprise expressing the nucleic acid sequences in an expression system suitable for expressing the antigen-binding protein so as to form an antigen-binding protein comprising a dimer of the heavy and light chain variable domains that bind the peptide-MHC class II complex.

In another embodiment, the method comprises isolating the nucleic acid from the non-human animal and obtaining an immunoglobulin heavy chain variable region sequence and/or an immunoglobulin light chain variable region sequence that encodes an immunoglobulin heavy chain variable domain and/or an immunoglobulin light chain variable domain, respectively, of an antibody that specifically binds the peptide-MHC class II complex. Such methods can further comprise employing the immunoglobulin heavy chain variable region sequence and/or the immunoglobulin light chain variable region sequence to produce an antibody that binds the peptide-MHC class II complex.

In some embodiments of the methods, cells (such as B cells) are recovered from the non-human animal (e.g., but not limited to, from spleen or lymph nodes). The cells may be fused with a myeloma cell line to prepare immortal hybridoma cell lines, and such hybridoma cell lines are screened and selected to identify hybridoma cell lines that produce antibodies containing hybrid heavy chains specific to the antigen used for immunization.

In some embodiments of the methods, immunization comprises priming (e.g., but not limited to, administering to) the non-human animal with the peptide-MHC class II complex, allowing the non-human animal to rest for a period of time, and re-immunizing (e.g., but not limited to, boosting the immune response of) the non-human animal with the peptide-MHC class II complex. In some embodiments of the methods, the methods comprise immunizing and/or boosting the non-human animal concomitantly with a helper T cell epitope, e.g., but not limited to, a pan DR T helper epitope (PADRE). See, e.g., U.S. Pat. No. 6,413,935 and Alexander et al. (1994) Immunity 1:751-61, each of which is incorporated herein by reference in its entirety for all purposes. In some embodiments of the methods, the method comprises priming the non-human animal with the peptide-MHC class II complex and boosting the immunized animal with the peptide-MHC class II complex linked to a helper T cell epitope, e.g., but not limited to, PADRE. In some embodiments of the methods, the method comprises both priming and boosting the non-human animal with the peptide-MHC class II complex linked to a helper T cell epitope. In methods comprising priming and/or boosting with PADRE, the non-human animal can be a mouse that comprises a C57/BL6 genetic background. In some embodiments, the mouse of a C57BL strain can be selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10C, and C57BL/Ola or can be a mix of an aforementioned C57BL/6 strain and another strain (e.g., but not limited to, 129, BALB, etc.). In some embodiments of the methods, the period of time between priming the non-human animal and boosting the non-human animal is a few days, at least a week, at least two weeks, at least three weeks, at least four weeks, or at least one month.

In some embodiments of the methods, the non-human animal can comprise human or humanized immunoglobulin heavy and/or light chain loci, such that the non-human animal is capable of providing human or humanized antigen-binding proteins comprising a human or humanized antigen-binding domain (e.g., but not limited to, human or humanized variable domains). Immunoglobulin loci comprising human variable region gene segments are known in the art and can be found, as non-limiting examples, in U.S. Pat. Nos. 5,633,425; 5,770,429; 5,814,318; 6,075,181; 6,114,598; 6,150,584; 6,998,514; 7,795,494; 7,910,798; 8,232,449; 8,502,018; 8,697,940; 8,703,485; 8,754,287; 8,791,323; 8,809,051; 8,907,157; 9,035,128; 9,145,588; 9,206,263; 9,447,177; 9,551,124; 9,580,491 and 9,475,559, each of which is herein incorporated by reference in its entirety for all purposes, as well as in U.S. Patent Publication Nos. 20100146647, 20110195454, 20130167256, 20130219535, 20130326647, 20130096287, and 20150113668, each of which is herein incorporated by reference in its entirety for all purposes, and in PCT Publication Nos. WO2007117410, WO2008151081, WO2009157771, WO2010039900, WO2011004192, WO2011123708 and WO2014093908, each of which is herein incorporated by reference in its entirety for all purposes. As non-limiting examples, the non-human animal can comprise in its genome an unrearranged or rearranged human or humanized immunoglobulin heavy locus and/or an unrearranged or rearranged human or humanized immunoglobulin light chain locus, such that the non-human animal is capable of providing human or humanized antigen-binding proteins comprising a human or humanized antigen-binding domain (e.g., but not limited to, human or humanized immunoglobulin variable domains), optionally, in some embodiments, wherein at least one of the human or humanized immunoglobulin heavy locus and/or human or humanized immunoglobulin light chain locus is unrearranged.

In some embodiments, such methods can further comprise cloning the nucleotide sequence encoding a human heavy or light chain variable region sequence (which may be encoding a histidine modified human heavy chain variable domain and/or a histidine modified human light chain variable domain, which may also or independently be a universal light chain variable domain) in frame with a gene encoding a human heavy chain constant region (CH) or light chain constant region (CL), to form a human binding protein sequence, and expressing the human binding protein sequence in a suitable cell.

Also provided are methods of identifying a T cell with specificity against an antigenic peptide or peptide-MHC class II complex, comprising immunizing a non-human animal with a peptide-MHC class II complex as described elsewhere herein, allowing the non-human animal to mount an immune response to the peptide MHC class II complex, and isolating a T cell reactive to the peptide or peptide-MHC class II complex.

Also provided are methods of making a nucleic acid sequence encoding a TCR variable domain (e.g., TCR α and/or β variable domains). In some embodiments, such methods can comprise immunizing a non-human animal with a peptide-MHC class II complex as described elsewhere herein, allowing the non-human animal to mount an immune response to the peptide MHC class II complex, and obtaining therefrom a nucleic acid sequence encoding a human TCR variable domain that binds the peptide or peptide-MHC class II complex. In one embodiment, the method can further comprise making a nucleic acid sequence encoding a TCR variable domain that is operably linked to a TCR constant region, comprising isolating a T cell from the non-human animal herein and obtaining therefrom the nucleic acid sequence encoding the TCR variable domain linked to a TCR constant region. In some embodiments, the non-human animal can comprise humanized T cell receptor variable gene loci, and the method can comprise determining a nucleic acid sequence of a human TCR variable region expressed by the T cell and cloning the human TCR variable region into a nucleotide construct comprising a nucleic acid sequence of a human TCR constant region such that the human TCR variable region is operably linked to the human TCR constant region. Optionally, in some embodiments, the method can further comprise expressing (e.g., in a cell) from the construct a human TCR specific for the peptide or peptide-MHC class II complex.

Also provided are methods of making a T cell receptor (TCR) specific for an antigenic peptide or peptide-MHC class II complex, comprising immunizing a non-human animal with a peptide-MHC class II complex as described elsewhere herein, allowing the non-human animal to mount an immune response to the peptide MHC class II complex, and isolating a T cell reactive to the peptide or peptide-MHC class II complex. In some embodiments, such methods can further comprise determining a nucleic acid sequence of a TCR variable region expressed by the T cell, cloning the TCR variable region into a nucleotide construct comprising a nucleic acid sequence of a TCR constant region such that the TCR variable region is operably linked to the TCR constant region, and optionally expressing from the construct (e.g., expressing in a cell) a TCR specific for peptide or peptide-MHC class II complex. In some embodiments, the non-human animal can comprise humanized T cell receptor variable gene loci, and the method can comprise determining a nucleic acid sequence of a human TCR variable region expressed by the T cell and cloning the human TCR variable region into a nucleotide construct comprising a nucleic acid sequence of a human TCR constant region such that the human TCR variable region is operably linked to the human TCR constant region. Optionally, in some embodiments, the method can further comprise expressing from the construct (e.g., in a cell) a human TCR specific for the peptide or peptide-MHC class II complex.

In some embodiments, the identified T cells or TCRs specific for the antigenic peptide or peptide-MHC class II complex can be used for therapy (e.g., adoptive T cell therapy) in a subject. In some embodiments, for example, such methods can comprise immunizing a non-human animal with a peptide-MHC class II complex as described elsewhere herein, allowing the non-human animal to mount an immune response to the peptide MHC class II complex, isolating a T cell (i.e., antigen-specific T cell) reactive to the peptide or peptide-MHC class II complex, determining the nucleic acid sequence of a TCR expressed by the T cell, cloning the nucleic acid sequence of the TCR into an expression vector (e.g., a retroviral vector), introducing the vector into T cells derived from the subject such that the T cells express the antigen-specific T cell receptor, and infusing the T cells into the subject. In some embodiments, an antigen-specific T cell population is expanded prior to infusing into the subject. In some embodiments, the subject's immune cell population is immunodepleted prior to the infusion of antigen-specific T cells.

In some embodiments of the methods, the non-human animal can express humanized T cell receptors. See, e.g., U.S. Pat. No. 9,113,616, herein incorporated by reference in its entirety for all purposes. As a non-limiting example, in some embodiments of the methods, the non-human animal can comprise humanized T cell receptor variable gene loci. As a non-limiting example, the non-human animal can express humanized TCR α and β polypeptides (and/or humanized TCRδ and TCRγ polypeptides). In one embodiment, the non-human animal comprises in its genome an unrearranged human TCR variable gene locus.

In some embodiments of the methods, the non-human animals are tolerized to at least one empty human or humanized MHC class II molecule, or at least the empty human peptide-binding groove thereof, but can generate antigen-binding proteins (e.g., but not limited to, antigen-binding proteins comprising human or humanized variable domains) to the human(ized) MHC molecule when it is complexed with an antigenic (e.g., but not limited to, heterologous) peptide. In some embodiments of the methods, tolerization of a non-human animal to an empty human(ized) MHC class II molecule is achieved by genetically modifying the non-human animal to comprise in its genome a nucleotide sequence encoding the human(ized) MHC molecule, or at least the human peptide-binding groove thereof, such that the non-human animal expresses the human(ized) MHC molecule, or at least the human peptide-binding groove thereof, as an empty human(ized) MHC molecule, or empty human peptide-binding groove thereof. The same animal genetically modified to comprise the nucleotide encoding the human(ized) MHC molecule may be further modified to comprise humanized immunoglobulin heavy and/or light chain loci that express human or humanized antigen-binding proteins (e.g., but not limited to, antigen-binding proteins having human or humanized variable domains) and/or may be further modified to comprise humanized T cell receptor variable gene loci. In some embodiments of the methods, the non-human animal comprises a nucleic acid encoding a human or humanized MHC II alpha polypeptide and/or a human or humanized MHC II beta polypeptide. See, e.g., US 2019/0292263, herein incorporated by reference in its entirety for all purposes. The MHC II nucleotide sequence may encode an MHC II protein that is fully human (e.g., but not limited to, a human HLA class II molecule) or a humanized MHC class II protein that is partially human and partially non-human (e.g., but not limited to, chimeric human/non-human MHC II protein, e.g., comprising chimeric human/non-human MHC II α and β polypeptides). A genetically modified non-human animal comprising in its genome (e.g., but not limited to, at the endogenous locus) a nucleotide sequence encoding humanized (e.g., but not limited to, chimeric human/non-human) MHC II polypeptides is disclosed in U.S. Pat. Nos. 8,847,005 and 9,043,996, each of which publications is incorporated herein by reference in its entirety for all purposes.

Although tolerization to human peptide-binding domains of chimeric human/non-human MHC molecules can be achieved by expression from endogenous MHC loci, in some embodiments such tolerization can also occur in non-human animals expressing a human MHC class II molecule (or the functional peptide-binding domain thereof) from an ectopic locus. Additionally, in some embodiments, non-human animals expressing and tolerized against an empty human MHC class II molecule (or the empty peptide-binding domain thereof) from an ectopic locus are able to generate a specific immune response against the human HLA molecule (or peptide binding-domain thereof, or derivative thereof) from which the expressed human MHC class II molecule is derived when the non-human animal is immunized with the human HLA molecule (or peptide binding-domain thereof and/or derivative thereof) complexed with an antigenic peptide (e.g., but not limited to, a peptide heterologous to the non-human animal). Without wishing to be bound by theory, it is believed that tolerization of the non-human animal occurs upon expression of the human or humanized MHC class II molecules. As such, it is not necessary that the human or humanized MHC class II molecules are expressed from an endogenous locus.

In some embodiments, such methods can further comprise breaking tolerance to endogenous peptides. Immunization of non-human animals (e.g., but not limited to, rodents, such as mice or rats) with an antigenic peptide-MHC class II complex to obtain specific peptide-MHC-class-II-binding proteins and peptide-MHC-class-II-specific T cells is dependent on a divergence in sequence between endogenous proteins in the non-human animal and the heterologous protein being presented to enable the non-human animal's immune system to recognize the peptide-MHC class II complex as non-self (i.e., foreign). The generation of antibodies and T cells/TCRs against peptide-MHC class II complexes having a high degree of homology with self-peptide-MHC class II complexes may be a difficult task in some cases due to immunological tolerance to self-peptide-MHC class II complexes. Methods of breaking tolerance to self-peptides that are homologous to a peptide of interest are well-known. See, e.g., US Publication No. 20170332610, herein incorporated by reference in its entirety for all purposes. In some embodiments of such methods, a method of breaking tolerance to endogenous peptides comprises modifying a non-human animal herein to comprise a deletion (e.g., but not limited to, a knockout mutation) of the self-peptide that has a high degree of homology with the peptide of interest.

A non-human animal in the methods disclosed in some embodiments herein can include, for example, any type of non-human animal, such as a mammal. Mammals include, for example, humans, non-human mammals, non-human primates, monkeys, apes, cats, dogs, horses, bulls, deer, bison, sheep, rabbits, rodents (e.g., but not limited to, mice, rats, hamsters, and guinea pigs), and livestock (e.g., but not limited to, bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human mammal” excludes humans. Particular non-limiting examples of non-human mammals include rodents, such as mice and rats.

All patent filings, websites, other publications, accession numbers and the like cited above or below are incorporated by reference in their entirety for all purposes to the same extent as if each individual item were specifically and individually indicated to be so incorporated by reference. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. When a DNA sequence encoding an amino acid sequence is provided, it is understood that RNA sequences that encode the same amino acid sequence are also provided (by replacing the thymines with uracils). The amino acid sequences follow the standard convention of beginning at the amino terminus of the sequence and proceeding forward (i.e., from left to right in each line) to the carboxy terminus.

TABLE 1.
Description of Sequences.

| SEQ | | |
|---|---|---|
| ID | | |
| NO | Type | Description |
| 1 | Protein | Linker v1 |
| 2 | Protein | Linker v2 |
| 3 | Protein | Linker v3 |
| 4 | Protein | Linker v4 |
| 5 | Protein | Linker v5 |
| 6 | Protein | Linker v6 |
| 7 | Protein | Linker v7 |
| 8 | Protein | Linker v8 |
| 9 | Protein | Linker v9 |
| 10 | Protein | Linker v10 |
| 11 | Protein | Linker v11 |
| 12 | Protein | Linker v12 |
| 13 | Protein | Linker v13 |
| 14 | Protein | Linker v14 |
| 15 | Protein | Linker v15 |
| 16 | Protein | Linker v16 |
| 17 | Protein | Linker v17 |
| 18 | Protein | Linker v18 |
| 19 | Protein | Linker v19 |
| 20 | Protein | Linker v20 |
| 21 | Protein | Linker v21 |
| 22 | Protein | TEV protease cleavage site |
| 23 | Protein | Fos leucine zipper motif |
| 24 | Protein | Jun leucine zipper motif |
| 25 | Protein | PADRE |
| 26 | Protein | LCMV epitope GP6-20 |
| 27 | Protein | LCMV epitope GP31-45 |
| 28 | Protein | LCMV epitope GP66-80 |
| 29 | Protein | LCMV epitope GP126-140 |
| 30 | Protein | LCMV epitope GP176-190 |
| 31 | Protein | LCMV epitope GP186-200 |
| 32 | Protein | LCMV epitope GP316-330 |
| 33 | Protein | LCMV epitope GP409-423 |
| 34 | Protein | LCMV epitope NP6-20 |
| 35 | Protein | LCMV epitope NP86-100 |
| 36 | Protein | LCMV epitope NP116-130 |
| 37 | Protein | LCMV epitope NP176-190 |
| 38 | Protein | LCMV epitope NP311-325 |
| 39 | Protein | LCMV epitope NP466-480 |
| 40 | Protein | LCMV epitope NP496-510 |
| 41 | Protein | LCMV epitope Z31-45 |
| 42 | Protein | BirA tag |
| 43 | Protein | mmH tag |
| 44 | Protein | QLQ peptide |
| 45 | Protein | FPQ peptide |
| 46 | Protein | PQP peptide |
| 47 | Protein | Linker v22 |
| 48 | Protein | mmH tag v2 |
| 49 | Protein | HLA class II histocompatibility antigen, DQ |
| | | alpha 1 chain (UniProt Accession No. P01909-1) |
| 50 | Protein | HLA class II histocompatibility antigen, DQ beta |
| | | 1 chain (NCBI Accession No. NP 001230891.1) |
| 51 | Protein | HLA class II histocompatibility antigen, DP alpha |
| | | 1 chain (UniProt Accession No. P20036-1) |
| 52 | Protein | HLA class II histocompatibility antigen, DR alpha |
| | | chain (UniProt Accession No. P01903-1) |
| 53 | Protein | HLA class II histocompatibility antigen, |
| | | DQ alpha 1 chain—R101C |
| 54 | Protein | HLA class II histocompatibility antigen, |
| | | DQ alpha 1 chain—C70A_R101C |
| 55 | Protein | HLA class II histocompatibility antigen, |
| | | DQ alpha 1 chain—C70Q |
| 56 | Protein | HLA class II histocompatibility antigen, |
| | | DQ alpha 1 chain—C70A |
| 57 | Protein | HLA class II histocompatibility antigen, |
| | | DQ alpha 1 chain—C70Q_R101C |
| 58 | Protein | HLA class II histocompatibility antigen, |
| | | DR alpha chain—F79C |
| 59 | Protein | HLA class II histocompatibility antigen, |
| | | DQ alpha 1 chain extracellular domain |
| 60 | Protein | HLA class II histocompatibility antigen, |
| | | DQ beta 1 chain extracellular domain |
| 61 | Protein | HLA class II histocompatibility antigen, |
| | | DP alpha 1 chain extracellular domain |
| 62 | Protein | HLA class II histocompatibility antigen, |
| | | DR alpha chain extracellular domain |
| 63 | Protein | HLA class II histocompatibility antigen, |
| | | DQ alpha 1 chain—R101C extracellular domain |
| 64 | Protein | HLA class II histocompatibility antigen, DQ |
| | | alpha 1 chain—C70A_R101C extracellular domain |
| 65 | Protein | HLA class II histocompatibility antigen, DQ alpha |
| | | 1 chain—C70Q extracellular domain |
| 66 | Protein | HLA class II histocompatibility antigen, DQ alpha |
| | | 1 chain—C70A extracellular domain |
| 67 | Protein | HLA class II histocompatibility antigen, DQ alpha |
| | | 1 chain—C70Q_R101C extracellular domain |
| 68 | Protein | HLA class II histocompatibility antigen, DR alpha |
| | | chain—F79C extracellular domain |
| 69 | Protein | Gliadin αII epitope |
| 70 | Protein | Gliadin ω2 epitope |
| 71 | Protein | Gliadin αI epitope P-1 to P9 |
| 72 | Protein | Gliadin αII epitope P-1 to P9 |
| 73 | Protein | Gliadin ω2 epitope P-1 to P9 |

### EXAMPLES

### Example 1. Design of Peptide-MHC II Protein Constructs

Examples of soluble peptide-MHC I protein constructs have been previously described. Such constructs can be used for various applications, including immunizing VELOCIMMUNE© rodents to generate anti-peptide-in-groove antibodies. This example describes the design of peptide-MHC II protein constructs in which the alpha and beta chains of the MHC II molecule are anchored together and to a peptide in its groove. These can be used for various applications, such as generation of soluble MHC II constructs to act as an immunogen as well as membrane-anchored MHC II proteins for other applications that include recruitment of T cells expressing MHC class II-peptide specific T-cell receptors (TCRs). Soluble or membrane-anchored MHC II proteins could also be used for specific targeting of T cells expressing MHC class II-peptide specific T-cell receptors (TCRs) to modulate T cell activity or viability in different disease settings.

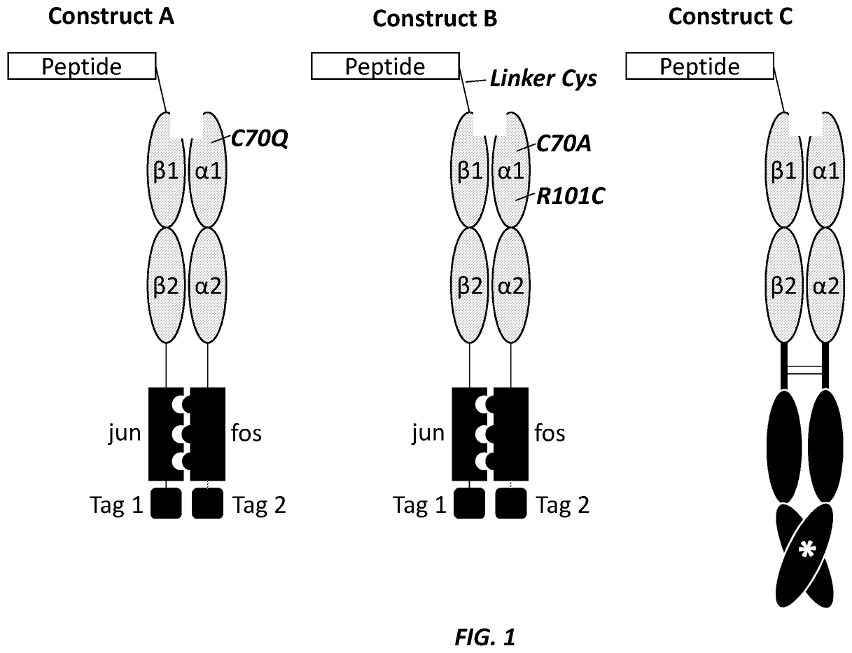

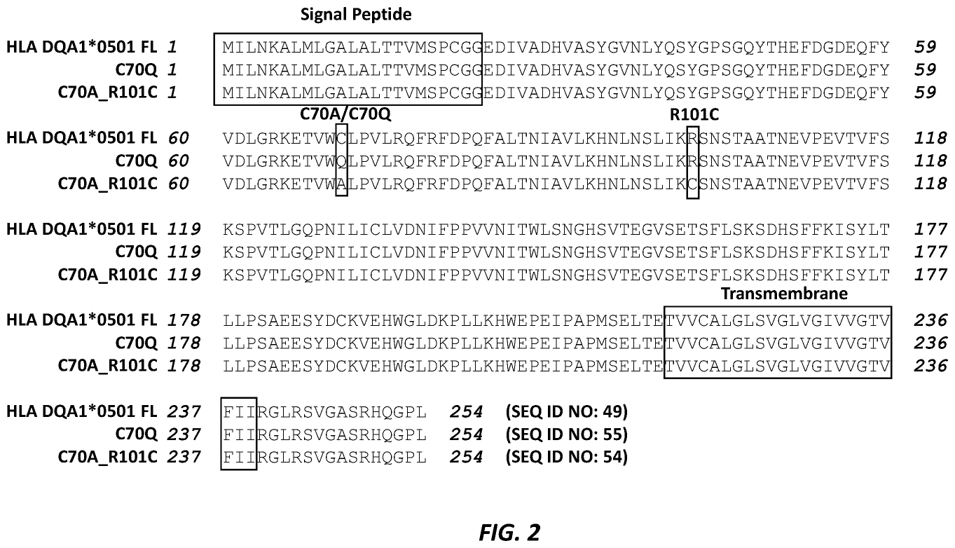

A variety of soluble peptide-MHC II constructs were designed as shown in FIG. 1. Descriptions of the soluble peptide-MHC II constructs are provided in Table 2. Some of the constructs include E. coli biotin ligase (BirA) and myc-myc-histidine (mmH) tags, but other tags (e.g., but not limited, to glutathione-s-transferase (GST), maltose binding protein (MBP), chitin binding protein (CBP), FLAG, or 1D4) can also be used. An alignment of the full-length DQ2 alpha chain segments used in the constructs (comprising either a C70Q mutation or both R101C and C70A mutations) is shown in FIG. 2. With respect to the C70 mutations, numbering may vary based on reference sequence or the chosen signal sequence for a given construct. C70 is the position within the full-length HLA-II DQ alpha 1 chain sequence designated as UniProt Accession No. P01909-1 (SEQ ID NO: 49). A version of the full-length HLA-II DQ alpha 1 chain sequence with the C70Q mutation is set forth in SEQ ID NO: 55, and a version of the full-length HLA-II DQ alpha 1 chain sequence with both the R101C and the C70A mutations is set forth in SEQ ID NO: 54. The portions of the full-length HLA-II DQ alpha 1 chain included in the soluble HLA-DQ2 constructs tested below included residues 24-216 of SEQ ID NO: 49 (no R101 or C70 mutation), SEQ ID NO: 55 (C70Q mutation), or SEQ ID NO: 54 (R101C and C70A mutations). A version of the full-length HLA-II DQ beta 1 chain sequence is designated as NCBI Accession No. NP_001230891.1 (SEQ ID NO: 50). The portions of the full-length HLA-II DQ beta 1 chain included in the soluble HLA-DQ2 constructs tested below included residues 33-230 of SEQ ID NO: 50. An alignment of full-length alpha chain segments from different HLA class II alleles is shown in FIG. 3.

TABLE 2
Soluble HLA-DQ2 Constructs.

| Construct | Description |
|---|---|
| A | MHC class II α and β extracellular domains linked to a |
| | Jun/Fos zipper |
| | MHC class II α extracellular domain with unpaired |
| | Cys mutation (C70Q) linked by an SGGGGG |
| | (SEQ ID NO: 1) linker to Fos zipper |
| | MHC class II β extracellular domains linked by an |
| | SGGGGG (SEQ ID NO: 1) linker to a Jun zipper |
| | Peptide is not disulfide-stapled (no Cys in the linker) |
| | Produced with QLQ peptide (purified yield of 14 mg/L) |
| B | MHC class II α and β extracellular domains linked to a |
| | Jun/Fos zipper |
| | MHC class II α extracellular domain with mutation |
| | in α (C70A, R101C) linked to Fos zipper |
| | MHC class II β extracellular domains linked by an |
| | SGGGGG (SEQ ID NO: 1) linker to a Jun zipper |
| | Disulfide staple between Cys in peptide-β linker and |
| | R101C mutation in α |
| | Produced with QLQ, FPQ, and PQP peptides (purified |
| | yields of 204, 36, and 0.9 mg/L, respectively) |
| C | MHC class II α and β extracellular domains linked to an |
| | immunoglobulin Fc domain comprising a Davis-body |
| | modification (*)—a CH3 modification that allows for |
| | differential binding of Fc to Protein A (see, e.g., U.S. |
| | Pat. No. 8,586,713, incorporated herein by reference |
| | in its entirely for all purposes) |
| | Unpaired Cys (C70) in MHC class II α intact |
| | Peptide is not disulfide stapled (no Cys in the linker) |
| | Produced with QLQ peptide (purified yield of 2.4 mg/L) |

Positive yields were observed with constructs A-C. Proteins were purified using standard procedures including affinity and size exclusion chromatography. Final protein amount obtained after purification was determined by UV absorbance and a calculated extinction coefficient based on amino acid composition of the protein. Production yields were calculated by dividing the mass of purified protein by volume of culture medium. Construct A produced a purified yield of 14 mg/L when covalently linked to the QLQPFPQPELPY (SEQ ID NO: 44, “QLQ” peptide) peptide. Construct C produced a purified yield of 2.4 mg/L when covalently linked to the QLQ peptide (SEQ ID NO: 44). Construct B was covalently linked to the QLQ peptide (SEQ ID NO: 44), FPQPEQPFPWQP (SEQ ID NO: 45; “FPQ” peptide), and PQPELPYPQPQL (SEQ ID NO: 46; “PQP” peptide) separately and produced purified yields of 204 mg/L, 36 mg/L, and 0.9 mg/L, respectively. A summary of the results is shown in Table 3.

Measurably consistent yields of soluble protein were produced by the peptide-MHC II construct containing:
(1) a jun/fos zipper at the C-terminus connected to a and β chains of MHC II with a SGGGGG (SEQ ID NO: 1) linker;
(2) an introduced R101C mutation in the α chain of MHC II;
(3) a linker at the N-terminus of the β chain connected to a peptide, where the linker includes an additional Cys mutation to allow the formation of a disulfide bond between the linker Cys and the introduced R101C mutation in the α chain of MHC II; and
(4) elimination of an unpaired Cys in the α chain (C70A mutation).

The unpaired Cys in DQA1*0501 is substituted by Trp, Arg, or Gln in the closest MHC sequences from other species based on sequence alignments, so mutations to these residues could instead be used.

TABLE 3
Soluble HLA-DQ2 Constructs-Purified Yields.

| | | | | | Ecto- | | Production | Purified |
|---|---|---|---|---|---|---|---|---|
| | Description | αCys | | Peptide | Jun/Fos | | Est Yield | Yield |
| Construct | (Peptide) | Mutation | Stapled | Linker | Linker | Produced | (mg/L) | (mg/L) |
| C | HLA-DQ2- | None | No | GGGGSENL | N/A | Yes | 12 | 2.4 |
| | Fc/Fc* | | | YFQGGGGS | | | | |
| | (QLQ) | | | (SEQ ID | | | | |
| | | | | NO: 47) | | | | |
| A | HLA-DQ2- | C70Q | No | GGGGSENL | SGGGGG | Yes | 30 | 14 |
| | jun/fos | | | YFQGGGGS | (SEQ ID | | | |
| | (QLQ) | | | (SEQ ID | NO: 1) | | | |
| | | | | NO: 47) | | | | |
| B | HLA-DQ2- | C70A, | Yes | GCGGSGG | SGGGGG | Yes | 200 (QLQ) | 204 (QLQ) |
| | jun/fos | R101C | | GGSGGGGS | (SEQ ID | | 100 (FPQ) | 36 (FPQ) |
| | (QLQ-, | | | (SEQ ID | NO: 1) | | 10 (PQP) | 0.9 (PQP) |
| | FPQ-, or | | | NO: 21) | | | | |
| | PQP- | | | | | | | |
| | stapled) | | | | | | | |

MHC constructs were tested for their ability to bind antibodies directed against MHC class II proteins via two different Biacore assays. In both assay formats, the instrument used was an Octet HTX, the chip type was either an anti-mouse or anti-human Fc coated Octet Biosensor, the assays were run at a temperature of 25° C., the running buffer was HBS-ET+1 mg/mL BSA, the capture mixing rate and time were 1000 rpm and 1 minute, and the sample injection mixing rate and time were 1000 rpm and 2 minutes.

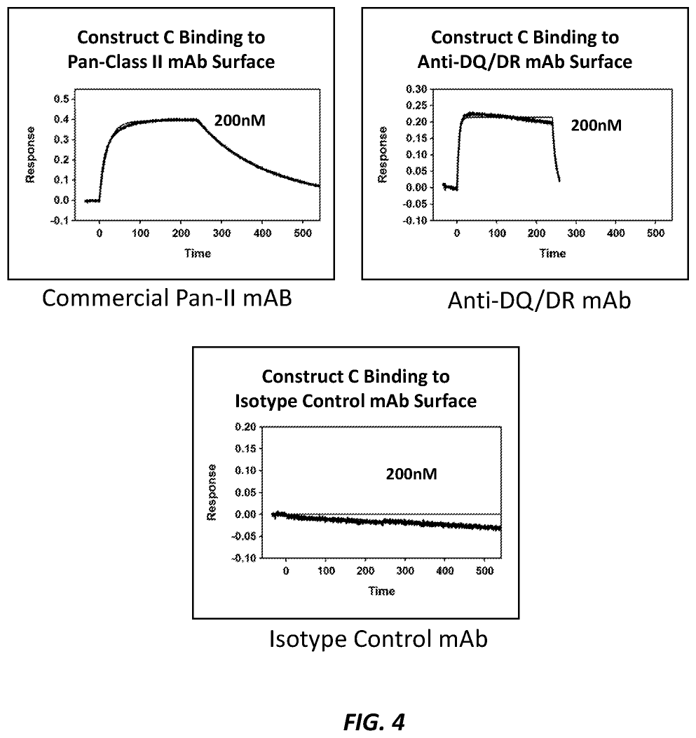

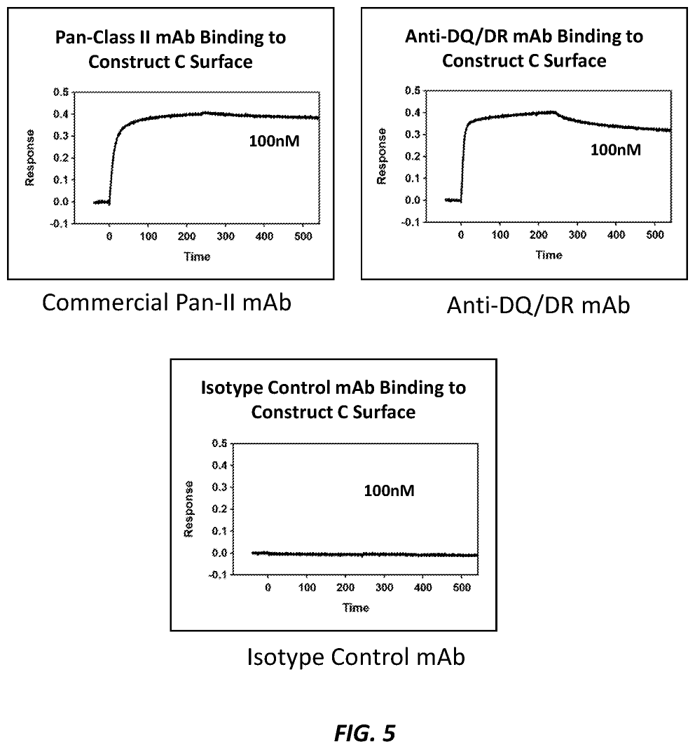

Construct C was analyzed to validate binding to monoclonal antibodies. In a first experiment, ˜0.8 nm of pan-class II anti-HLA mAb or anti-DR/DQ mAb was captured by dipping anti-mFc coated Octet biosensor in wells containing 100 nM of mAb for 1 minute. mAb-captured sensors were then submerged in wells containing 200 nM of construct C. As shown in FIG. 4 and Table 4, soluble construct C bound to both anti-class II monoclonal antibodies captured on an anti-mFc sensor surface but not to the isotype control mAb. In a second experiment, ˜1 nm of construct C was captured by dipping anti-hFc coated Octet biosensor in wells containing 200 nM of construct C for 1 minute. Construct-C-captured sensors were then submerged in wells containing 100 nM of pan-class II anti-HLA mAb or anti-DR/DQ mAb. As shown in FIG. 5 and Table 5, soluble construct C captured on an anti-hFc sensor surface bound to both anti-class II monoclonal antibodies but not to isotype control mAb. The pan-class II anti-HLA antibodies bind only to HLA protein that is properly folded, which enabled us to validate the conformational integrity of the protein that was produced and purified.

TABLE 4
Construct C Binds to Anti-Class II mAbs Captured on Anti-mFc Sensor Surface.

| | mAb | 200 nM | | | | | |
|---|---|---|---|---|---|---|---|
| | Capture | Construct | | | | | |
| | Level | C Bound | ka | kd | KD | t½ | Rm x |
| mAb Captured | (nm) | (nm) | (1/Ms) | (1/s) | (M) | (min) | (RU) |
| Pan-class II | 0.80 | 0.39 | 2.34E+05 | 5.74E−03 | 2.45E−08 | 2.0 | 0.44 |
| anti-HLA mAb | | | | | | | |
| anti-DR/DQ | 0.99 | 0.19 | 5.09E+05 | 1.11E−01 | 2.19E−07 | 0.1 | 0.45 |
| mAb | | | | | | | |
| Isotype Control | 1.07 | −0.02 | NB (no | NB | NB | NB | NB |
| mAb | | | binding) | | | | |

TABLE 5
Construct C Captured on Anti-hFc Sensor Surface
Binds to Anti-Class II mAbs.

| | Construct C | 100 nM mAb |
|---|---|---|
| mAb Captured | Capture Level (nm) | Bound (nm) |
| Pan-class II anti-HLA mAb | 1.02 | 0.40 |
| anti-DR/DQ mAb | 0.94 | 0.40 |
| Isotype Control mAb | 0.98 | −0.01 |

Construct B is analyzed to validate binding to monoclonal antibodies. In a first experiment, ˜0.8 nm of pan-class II anti-HLA mAb or anti-DR/DQ mAb is captured by dipping anti-mFc coated Octet biosensor in wells containing 100 nM of mAb for 1 minute. mAb-captured sensors are then submerged in wells containing 200 nM of construct B. Soluble construct B binds to both anti-class II monoclonal antibodies captured on an anti-mFc sensor surface but not to the isotype control mAb. In a second experiment, ˜1 nm of construct B is captured by dipping anti-hFc coated Octet biosensor in wells containing 200 nM of construct B for 1 minute. Construct-B-captured sensors are then submerged in wells containing 100 nM of pan-class II anti-HLA mAb or anti-DR/DQ mAb. Soluble construct B captured on an anti-hFc sensor surface binds to both anti-class II monoclonal antibodies but not to isotype control mAb. The pan-class II anti-HLA antibodies bind only to HLA protein that is properly folded, which enables us to validate the conformational integrity of the protein that is produced and purified.

### Example 2. Tolerization of Mice

Mice are generated or provided that are tolerized to an empty MHC class II molecule, where the MHC class II molecule is not derived from a mouse (e.g., but not limited to, human). For example, first mice expressing an MHC class II molecule from the corresponding endogenous locus or a locus other than the corresponding endogenous locus (e.g., but not limited to, the ROSA26 locus) are tolerized to an empty MHC class II molecule. Then, these tolerized mice are injected with an immunogen (e.g., an MHC class II molecule that includes an immunogenic peptide in the groove such as Construct A, Construct B, or Construct C from Example 1). These immunized mice produce specific antibody titers to this specific immunogen when compared with mice that are not tolerized to an empty MHC class II molecule. Instead, mice that are not tolerized and immunized with the subject MHC class II molecule generate antibodies which recognize not only the immunogenic peptide, but also recognize the MHC class II molecule. Accordingly, tolerized mice which are immunized with the MHC class II molecule described herein are able to generate immune responses specific to the antigen without generating antibodies to the MHC class II molecule alone.

### Example 3. Immunization of a Tolerized Mouse with MHC Protein Constructs

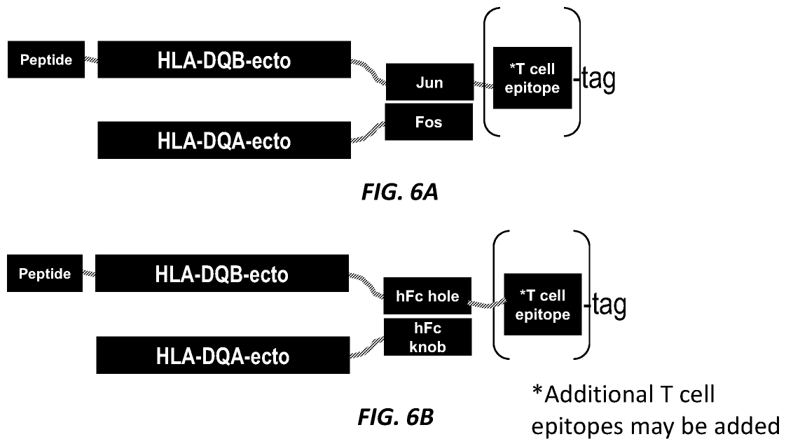

Peptides for tethering to the HLA-DQB chain as DNA and soluble dimer proteins are chosen for immunization and screening. Schematics of examples of constructs with peptides tethered to the HLA-DQB chain for immunization and screening are shown in FIGS. 6A and 6B.

Mice (e.g., mice comprising humanized immunoglobulin heavy and/or light chain variable region loci) are immunized with a peptide-MHC (pMIC) complex of interest that comprises a peptide that is antigenic to the mice and the human or humanized MHC class II molecule against which the mouse is tolerized. Mice are additionally and optionally boosted with the pMHC complex of interest, which booster is also optionally linked to a helper T cell epitope. Antibodies (e.g., human or humanized antibodies expressed from humanized immunoglobulin heavy and/or light chain loci) are isolated from immunized mice and are tested for specificity of binding to the pMHC complex.

Test mice that are tolerized to a human MHC II molecule and comprise nucleotide sequences encoding a humanized immunoglobulin heavy chain locus (see, e.g., Macdonald (2014) Proc. Natl. Acad. Sci. U.S.A. 111:5147-5152, herein incorporated by reference in its entirety for all purposes) and a humanized common light chain locus (see, e.g., U.S. Pat. Nos. 10,143,186; 10,130,081 and 9,969,814; U.S. Patent Pub. Nos. 20120021409, 20120192300, 20130045492, 20130185821, 20130302836, and 20150313193, each of which is incorporated by reference in its entirety for all purposes) are provided. These test mice, and non-tolerized control mice comprising a functional (e.g., murine) ADAM6 gene (see, e.g., U.S. Pat. Nos. 8,642,835 and 8,697,940; each of which is incorporated by reference in its entirety for all purposes) and humanized immunoglobulin heavy and light chain loci are immunized with a pMHC complex comprising a heterologous peptide-in-groove presented in the context of an HLA-DQ molecule, where the immunogen is administered as a protein immunogen or as DNA encoding the pMHC complex. The mice are boosted via different routes at varying time intervals using the pMHC complex immunogen with standard adjuvants or using the pMHC complex immunogen linked to a T helper Pan-DR epitope (PADRE) peptide. Pre-immune serum is collected from the mice prior to the initiation of immunization. The mice are bled periodically and anti-serum titers are assayed on respective antigens.

Antibody titers in serum against irrelevant antigens (i.e., antigens that the mice have not seen and thus are not expected to elicit a significant response upon titering) and relevant antigens presented in the context of HLA-DQ (peptide-in-groove) are determined using ELISA. Ninety-six well microtiter plates (Thermo Scientific) are coated with tagged pMHC complexes comprising relevant peptide-in-groove or irrelevant antigens presented in the context of the HLA-DQ in phosphate-buffered saline (PBS, Irvine Scientific) overnight. Plates are washed with phosphate-buffered saline containing 0.05% Tween 20 (PBS-T, Sigma-Aldrich) and are blocked with bovine serum albumin (BSA, Sigma-Aldrich) in PBS.

Pre-immune and immune anti-sera are serially diluted in BSA-PBS and added to the plates. The plates are washed, and anti-mouse IgG-Fc-Horse Radish Peroxidase (HRP) conjugated secondary antibody is added to the plates. Plates are washed and developed using 3,3′,5,5′-Tetramethylbenzidine (TMB)/H2O2 as substrate according to manufacturer's recommended procedure, and absorbance at 450 nm is recorded using a spectrophotometer (Victor, Perkin Elmer). Antibody titers are computed using Graphpad PRISM software. Antibody titers are calculated as the interpolated serum dilution factor of which the binding signal is 2-fold over background.

Tolerizing a mouse to human HLA class II molecules or portions thereof enhances the ability of the mouse to generate specific antibody responses to a pMHC of interest compared to control mice that are not tolerized to the human HLA class II molecule.

### Example 4. Testing Different Peptides in Peptide-MHC II Protein Constructs

A variety of soluble peptide-MHC II constructs with different variations of gliadin immunogens were designed to test varying the parameters for the MHC ligand peptide and to confirm expression of the constructs with different ligand peptides. Specifically, variations of αI gliadin, αII gliadin, and ω2 gliadin (Table 6) were tested.

TABLE 6
Gliadin Epitopes.

| Epitope | P-3 | P-2 | P-1 | P1 | P2 | P3 | P4 | P5 | P6 | P7 | P8 | P9 |
|---|---|---|---|---|---|---|---|---|---|---|---|---|
| αI (SEQ ID | Q | L | Q | P | F | P | Q | P | E | L | P | Y |
| NO: 44) | | | | | | | | | | | | |
| αII (SEQ ID | Q | P | F | P | Q | P | E | L | P | Y | P | Q |
| NO: 69) | | | | | | | | | | | | |
| ω2 (SEQ ID | Q | P | F | P | Q | P | E | Q | P | F | P | W |
| NO: 70) | | | | | | | | | | | | |

The portions of the full-length HLA-II DQ alpha 1 chain included in the soluble HLA-DQ2 constructs tested below included residues 24-216 of SEQ ID NO: 54 (R101C and C70A mutations; SEQ ID NO: 64). The portions of the full-length HLA-II DQ beta 1 chain included in the soluble HLA-DQ2 constructs tested below included residues 33-230 of SEQ ID NO: 50 (SEQ ID NO: 60). Descriptions of the soluble peptide-MHC II constructs are provided in Table 7. Some of the constructs included PADRE, but other T cell epitopes can also be used. As shown in Table 7, positive yields were observed with all constructs.

TABLE 7
Soluble HLA-DQ2 Constructs.

| | | Yield |
|---|---|---|
| Gliadin Peptide | Construct Description | (mg) |
| αI-gliadin, | MHC class II α and β extracellular domains linked to a Jun/Fos zipper | 102 |
| P-3 to P9: | MHC class II α extracellular domain with mutation in α (C70A, R101C; | |
| QLQPFPQPELPY | SEQ ID NO: 64) linked to Fos zipper (SEQ ID NO: 23) | |
| (SEQ ID NO: 44) | MHC class II β extracellular domain (SEQ ID NO: 60) linked by an | |
| | SGGGGG (SEQ ID NO: 1) linker to a Jun zipper (SEQ ID NO: 24) | |
| | Disulfide staple between Cys in peptide-β linker | |
| | (GCGGSGGGGSGGGGS; SEQ ID NO: 21) and R101C mutation in α | |
| αI-gliadin, | MHC class II α and β extracellular domains linked to a Jun/Fos zipper | 1.35 |
| P-1 to P9: | MHC class II α extracellular domain with mutation in α (C70A, R101C; | |
| QPFPQPELPY | SEQ ID NO: 64) linked to Fos zipper (SEQ ID NO: 23) | |
| (SEQ ID NO: 71) | MHC class II β extracellular domain (SEQ ID NO: 60) linked by an | |
| | SGGGGG (SEQ ID NO: 1) linker to a Jun zipper (SEQ ID NO: 24) which is | |
| | further linked by a GGGGSGGGGS (SEQ ID NO: 14) linker to PADRE | |
| | (SEQ ID NO: 25) | |
| | Disulfide staple between Cys in peptide-β linker | |
| | (GCGGSGGGGSGGGGS; SEQ ID NO: 21) and R101C mutation in α | |
| αII-gliadin, | MHC class II α and β extracellular domains linked to a Jun/Fos zipper | 0.4 |
| P1 to P12: | MHC class II α extracellular domain with mutation in α (C70A, R101C; | |
| PQPELPYPQPQL | SEQ ID NO: 64) linked to Fos zipper (SEQ ID NO: 23) | |
| (SEQ ID NO: 46) | MHC class II P extracellular domain (SEQ ID NO: 60) linked by an | |
| | SGGGGG (SEQ ID NO: 1) linker to a Jun zipper (SEQ ID NO: 24) | |
| | Disulfide staple between Cys in peptide-β linker | |
| | (GCGGSGGGGSGGGGS; SEQ ID NO: 21) and R101C mutation in α | |
| αII-gliadin, | MHC class II α and β extracellular domains linked to a Jun/Fos zipper | 2.1 |
| P-1 to P9: | MHC class II α extracellular domain with mutation in α (C70A, R101C; | |
| FPQPELPYPQ | SEQ ID NO: 64) linked to Fos zipper (SEQ ID NO: 23) | |
| (SEQ ID NO: 72) | MHC class II β extracellular domain (SEQ ID NO: 60) linked by an | |
| | SGGGGG (SEQ ID NO: 1) linker to a Jun zipper (SEQ ID NO: 24) which is | |
| | further linked by a GGGGSGGGGS (SEQ ID NO: 14) linker to PADRE | |
| | (SEQ ID NO: 25) | |
| | Disulfide staple between Cys in peptide-β linker | |
| | (GCGGSGGGGSGGGGS; SEQ ID NO: 21) and R101C mutation in α | |
| ωII-gliadin, | MHC class II α and β extracellular domains linked to a Jun/Fos zipper | 18 |
| P-1 to P11: | MHC class II α extracellular domain with mutation in α (C70A, R101C; | |
| FPQPEQPFPWQP | SEQ ID NO: 64) linked to Fos zipper (SEQ ID NO: 23) | |
| (SEQ ID NO: 45) | MHC class II β extracellular domain (SEQ ID NO: 60) linked by an | |
| | SGGGGG (SEQ ID NO: 1) linker to a Jun zipper (SEQ ID NO: 24) | |
| | Disulfide staple between Cys in peptide-β linker | |
| | (GCGGSGGGGSGGGGS; SEQ ID NO: 21) and R101C mutation in α | |
| ωII-gliadin, | MHC class II α and β extracellular domains linked to a Jun/Fos zipper | 2 |
| P-1 to P9: | MHC class II β extracellular domain with mutation in α (C70A, R101C; | |
| FPQPEQPFPW | SEQ ID NO: 64) linked to Fos zipper (SEQ ID NO: 23) | |
| (SEQ ID NO: 73) | MHC class II β extracellular domain (SEQ ID NO: 60) linked by an | |
| | SGGGGG (SEQ ID NO: 1) linker to a Jun zipper (SEQ ID NO: 24) which is | |
| | further linked by a GGGGSGGGGS (SEQ ID NO: 14) linker to PADRE | |
| | (SEQ ID NO: 25) | |
| | Disulfide staple between Cys in peptide-β linker | |
| | (GCGGSGGGGSGGGGS; SEQ ID NO: 21) and R101C mutation in α | |

Proteins are purified using standard procedures including affinity and size exclusion chromatography. Final protein amount obtained after purification is determined by UV absorbance and a calculated extinction coefficient based on amino acid composition of the protein. Production yields are calculated by dividing the mass of purified protein by volume of culture medium.

Peptide-MHC constructs are tested for their ability to bind antibodies directed against MHC class II proteins via two different Biacore assays. In both assay formats, the instrument used is an Octet HTX, the chip type is either an anti-mouse or anti-human Fc coated Octet Biosensor, the assays are run at a temperature of 25° C., the running buffer is HBS-ET+1 mg/mL BSA, the capture mixing rate and time are 1000 rpm and 1 minute, and the sample injection mixing rate and time are 1000 rpm and 2 minutes.

Each construct is analyzed to validate binding to monoclonal antibodies. In a first experiment, ˜0.8 nm of pan-class II anti-HLA mAb or anti-DR/DQ mAb is captured by dipping anti-mFc coated Octet biosensor in wells containing 100 nM of mAb for 1 minute. mAb-captured sensors are then submerged in wells containing 200 nM of the peptide-MHC construct. The soluble peptide-MHC construct binds to both anti-class II monoclonal antibodies captured on an anti-mFc sensor surface but not to the isotype control mAb. In a second experiment, ˜1 nm of peptide-MHC construct is captured by dipping anti-hFc coated Octet biosensor in wells containing 200 nM of the peptide-MHC construct for 1 minute. Peptide-MHC-construct-captured sensors are then submerged in wells containing 100 nM of pan-class II anti-HLA mAb or anti-DR/DQ mAb. Soluble peptide-MHC construct captured on an anti-hFc sensor surface binds to both anti-class II monoclonal antibodies but not to isotype control mAb. The pan-class II anti-HLA antibodies bind only to HLA protein that is properly folded, which enables us to validate the conformational integrity of the protein that is produced and purified.

Mice are then generated or provided that are tolerized to an empty MHC class II molecule, where the MHC class II molecule is not derived from a mouse (e.g., but not limited to, human). For example, first mice expressing an MHC class II molecule from the corresponding endogenous locus or a locus other than the corresponding endogenous locus (e.g., but not limited to, the ROSA26 locus) are tolerized to an empty MHC class II molecule. Then, these tolerized mice are injected with an immunogen (e.g., an MHC class II molecule that includes an immunogenic peptide in the groove such as any of the peptide-MHC constructs in Example 4). These immunized mice produce specific antibody titers to this specific immunogen when compared with mice that are not tolerized to an empty MHC class II molecule. Instead, mice that are not tolerized and immunized with the subject MHC class II molecule generate antibodies which recognize not only the immunogenic peptide, but also recognize the MHC class II molecule. Accordingly, tolerized mice which are immunized with the MHC class II molecule described herein are able to generate immune responses specific to the antigen without generating antibodies to the MHC class II molecule alone.

Peptides, such as those described in Example 4, for tethering to the HLA-DQB chain as DNA and soluble dimer proteins are chosen for immunization and screening.

Mice (e.g., mice comprising humanized immunoglobulin heavy and/or light chain variable region loci) are immunized with a peptide-MHC (pMHC) complex of interest that comprises a peptide that is antigenic to the mice and the human or humanized MHC class II molecule against which the mouse is tolerized. Mice are additionally and optionally boosted with the pMHC complex of interest, which booster is also optionally linked to a helper T cell epitope. Antibodies (e.g., human or humanized antibodies expressed from humanized immunoglobulin heavy and/or light chain loci) are isolated from immunized mice and are tested for specificity of binding to the pMHC complex.

Test mice that are tolerized to a human MHC II molecule and comprise nucleotide sequences encoding a humanized immunoglobulin heavy chain locus (see, e.g., Macdonald (2014) Proc. Natl. Acad. Sci. U.S.A. 111:5147-5152, herein incorporated by reference in its entirety for all purposes) and a humanized common light chain locus (see, e.g., U.S. Pat. Nos. 10,143,186; 10,130,081 and 9,969,814; U.S. Patent Pub. Nos. 20120021409, 20120192300, 20130045492, 20130185821, 20130302836, and 20150313193, each of which is incorporated by reference in its entirety for all purposes) are provided. These test mice, and non-tolerized control mice comprising a functional (e.g., murine) ADAM6 gene (see, e.g., U.S. Pat. Nos. 8,642,835 and 8,697,940; each of which is incorporated by reference in its entirety for all purposes) and humanized immunoglobulin heavy and light chain loci are immunized with a pMHC complex comprising a heterologous peptide-in-groove presented in the context of an HLA-DQ molecule, where the immunogen is administered as a protein immunogen or as DNA encoding the pMHC complex. The mice are boosted via different routes at varying time intervals using the pMHC complex immunogen with standard adjuvants or using the pMHC complex immunogen linked to a T helper Pan-DR epitope (PADRE) peptide. Pre-immune serum is collected from the mice prior to the initiation of immunization. The mice are bled periodically and anti-serum titers are assayed on respective antigens.

Antibody titers in serum against irrelevant antigens (i.e., antigens that the mice have not seen and thus are not expected to elicit a significant response upon titering) and relevant antigens presented in the context of HLA-DQ (peptide-in-groove) are determined using ELISA. Ninety-six well microtiter plates (Thermo Scientific) are coated with tagged pMHC complexes comprising relevant peptide-in-groove or irrelevant antigens presented in the context of the HLA-DQ in phosphate-buffered saline (PBS, Irvine Scientific) overnight. Plates are washed with phosphate-buffered saline containing 0.05% Tween 20 (PBS-T, Sigma-Aldrich) and are blocked with bovine serum albumin (BSA, Sigma-Aldrich) in PBS.

Pre-immune and immune anti-sera are serially diluted in BSA-PBS and added to the plates. The plates are washed, and anti-mouse IgG-Fc-Horse Radish Peroxidase (HRP) conjugated secondary antibody is added to the plates. Plates are washed and developed using 3,3′,5,5′-Tetramethylbenzidine (TMB)/H₂O₂ as substrate according to manufacturer's recommended procedure, and absorbance at 450 nm is recorded using a spectrophotometer (Victor, Perkin Elmer). Antibody titers are computed using Graphpad PRISM software. Antibody titers are calculated as the interpolated serum dilution factor of which the binding signal is 2-fold over background.

Tolerizing a mouse to human HLA class II molecules or portions thereof enhances the ability of the mouse to generate specific antibody responses to a pMHC of interest compared to control mice that are not tolerized to the human HLA class II molecule.

We claim:

1. A composition comprising an MHC ligand peptide covalently attached to an MHC class II molecule comprising an MHC class II α chain or a portion thereof and an MHC class II β chain or a portion thereof,
wherein the MHC ligand peptide is covalently attached to the MHC class II molecule by a peptide linker,
wherein the MHC ligand peptide or the peptide linker comprises a first cysteine and the MHC class II molecule comprises a second cysteine, and
wherein the first cysteine and the second cysteine form a disulfide bond such that the MHC ligand peptide is bound in a peptide-binding groove formed by the MHC class II α chain or the portion thereof and the MHC class II β chain or the portion thereof.

2. The composition of claim 1, wherein the MHC class II α chain or the portion thereof comprises an α1 domain, and the MHC class II β chain or the portion thereof comprises a β1 domain.

3. The composition of claim 2, wherein the MHC class II α chain or the portion thereof comprises an MHC class II α chain extracellular domain, and the MHC class IIR chain or the portion thereof comprises an MHC class II β chain extracellular domain.

4. The composition of claim 2 or 3, wherein:
(1) the MHC class II α chain or the portion thereof comprises the α1 domain, an α2 domain, a transmembrane domain, and a cytoplasmic domain; and
(2) the MHC class II β chain or the portion thereof comprises the β1 domain, a β2 domain, a transmembrane domain, and a cytoplasmic domain.

5. The composition of any preceding claim, wherein the composition is membrane-anchored.

6. The composition of any one of claims 1-3, wherein the composition is soluble.

7. The composition of claim 6, wherein:
(1) the MHC class II α chain or the portion thereof comprises the α1 domain and an α2 domain but does not comprise a transmembrane domain or a cytoplasmic domain; and
(2) the MHC class II β chain or the portion thereof comprises the β1 domain and a β2 domain but does not comprise a transmembrane domain or a cytoplasmic domain.

8. The composition of claim 6 or 7, wherein the MHC class II α chain or the portion thereof and the MHC class II β chain or the portion thereof are linked by a Jun-Fos zipper, electrostatic engineering, knobs-into-holes, an immunoglobulin scaffold, an immunoglobulin Fc region, or a linker.

9. The composition of claim 8, wherein the MHC class II α chain or the portion thereof and the MHC class II β chain or the portion thereof are linked by a Jun-Fos zipper comprising a Jun leucine zipper dimerization motif and a Fos leucine zipper dimerization motif, and wherein
the MHC class II α chain or the portion thereof is linked to the Jun leucine zipper dimerization motif and the MHC class II β chain or the portion thereof is linked to the Fos leucine zipper dimerization motif, or the MHC class II α chain or the portion thereof is linked to the Fos leucine zipper dimerization motif and the MHC class II β chain or the portion thereof is linked to the Jun leucine zipper dimerization motif.

10. The composition of claim 9, wherein the C-terminal end of the MHC class II α chain or the portion thereof is linked to the Jun leucine zipper dimerization motif and the C-terminal end of the MHC class II β chain or the portion thereof is linked to the Fos leucine zipper dimerization motif, or
wherein the C-terminal end of the MHC class II α chain or the portion thereof is linked to the Fos leucine zipper dimerization motif and the C-terminal end of the MHC class II R chain or the portion thereof is linked to the Jun leucine zipper dimerization motif.

11. The composition of claim 9 or 10, wherein the MHC class II α chain or the portion thereof is linked to the Jun leucine zipper dimerization motif by an MHC-Jun linker, and the MHC class II β chain or the portion thereof is linked to the Fos leucine zipper dimerization motif by an MHC-Fos linker, or
wherein the MHC class II α chain or the portion thereof is linked to the Fos leucine zipper dimerization motif by the MHC-Fos linker, and the MHC class II β chain or the portion thereof is linked to the Jun leucine zipper dimerization motif by the MHC-Jun linker.

12. The composition of claim 11, wherein the MHC-Jun linker and the MHC-Fos linker each comprise the sequence set forth in SEQ ID NO: 1.

13. The composition of any preceding claim, wherein the MHC ligand peptide is about 10 to about 18 amino acids in length, is about 10 to about 15 amino acids in length, or is about 10 to about 12 amino acids in length, or
wherein the MHC ligand peptide comprises residues P-1 to P9 or residues P-3 to P9.

14. The composition of any preceding claim, wherein the MHC ligand peptide is an antigenic MHC ligand peptide.

15. The composition of any preceding claim, wherein the MHC ligand peptide is associated with a T-cell-mediated disease.

16. The composition of any preceding claim, wherein the peptide linker linking the MHC ligand peptide to the MHC class II molecule is a flexible linker.

17. The composition of any preceding claim, wherein the peptide linker linking the MHC ligand peptide to the MHC class II molecule comprises one or more flexible amino acids and one or more polar amino acids.

18. The composition of any preceding claim, wherein the peptide linker linking the MHC ligand peptide to the MHC class II molecule does not comprise any charged amino acids.

19. The composition of any preceding claim, wherein the peptide linker linking the MHC ligand peptide to the MHC class II molecule comprises a cleavage site.

20. The composition of claim 19, wherein the cleavage site is a tobacco etch virus (TEV) protease cleavage site.

21. The composition of any preceding claim, wherein the peptide linker linking the MHC ligand peptide to the MHC class II molecule is non-immunogenic.

22. The composition of any preceding claim, wherein the peptide linker linking the MHC ligand peptide to the MHC class II molecule is connected to the N-terminal end of the MHC class II β chain or the portion thereof.

23. The composition of any preceding claim, wherein the peptide linker linking the MHC ligand peptide to the MHC class II molecule is connected to the N-terminal end of the MHC class II α chain or the portion thereof.

24. The composition of any preceding claim, wherein the peptide linker linking the MHC ligand peptide to the MHC class II molecule is at least about 9 amino acids in length.

25. The composition of any preceding claim, wherein the peptide linker linking the MHC ligand peptide to the MHC class II molecule is between about 9 and about 50 amino acids in length.

26. The composition of any preceding claim, wherein the peptide linker linking the MHC ligand peptide to the MHC class II molecule comprises 2-4 repeats of the sequence set forth in SEQ ID NO: 4.

27. The composition of any preceding claim, wherein the peptide linker linking the MHC ligand peptide to the MHC class II molecule comprises the first cysteine.

28. The composition of claim 27, wherein the first cysteine is the only cysteine in the peptide linker linking the MHC ligand peptide to the MHC class II molecule.

29. The composition of claim 27 or 28, wherein the first cysteine is in the first four amino acids of the peptide linker linking the MHC ligand peptide to the MHC class II molecule.

30. The composition of any preceding claim, wherein the peptide linker linking the MHC ligand peptide to the MHC class II molecule comprises 2-4 repeats of the sequence set forth in SEQ ID NO: 4, wherein one amino acid in one of the repeats is mutated to cysteine.

31. The composition of claim 30, wherein the peptide linker linking the MHC ligand peptide to the MHC class II molecule comprises the sequence set forth in SEQ ID NO: 21.

32. The composition of any one of claims 1-26, wherein the MHC ligand peptide comprises the first cysteine.

33. The composition of claim 32, wherein the first cysteine faces away from an epitope formed by the composition.

34. The composition of any preceding claim, wherein the second cysteine is in the MHC class II α chain or the portion thereof.

35. The composition of claim 34, wherein the peptide linker linking the MHC ligand peptide to the MHC class II molecule is connected to the N-terminal end of the MHC class II β chain or the portion thereof.

36. The composition of any preceding claim, wherein the second cysteine is not present in a wild type MHC class II molecule corresponding to the MHC class II molecule in the composition.

37. The composition of claim 36, wherein the second cysteine is in place of a non-cysteine amino acid in the corresponding wild type MHC class II molecule.

38. The composition of claim 37, wherein the second cysteine is in the MHC class II α chain or the portion thereof, and
wherein the second cysteine is at a position corresponding to position 101 in the sequence set forth in SEQ ID NO: 49 when the MHC class II α chain or the portion thereof is optimally aligned with SEQ ID NO: 49.

39. The composition of any preceding claim, wherein the MHC class II molecule lacks a cysteine present in a corresponding wild type MHC class II molecule.

40. The composition of claim 39, wherein the cysteine present in the corresponding wild type MHC class II molecule has been replaced with an alanine or a glutamine in the MHC class II molecule in the composition.

41. The composition of any one of claims 1-38, wherein the MHC class II α chain or the portion thereof lacks a cysteine present in a corresponding wild type MHC class II α chain.

42. The composition of claim 41, wherein the cysteine present in the corresponding wild type MHC class II α chain has been replaced with an alanine or a glutamine in the MHC class II α chain or the portion thereof in the composition.

43. The composition of claim 41 or 42, wherein the cysteine in the corresponding wild type MHC class II α chain is at a position corresponding to position 70 in the sequence set forth in SEQ ID NO: 49 when the MHC class II α chain or the portion thereof is optimally aligned with SEQ ID NO: 49.

44. The composition of any preceding claim, wherein the composition further comprises one or more immunostimulatory molecules.

45. The composition of claim 44, wherein the one or more immunostimulatory molecules comprise a pan-DR-binding epitope (PADRE) and/or a peptide from lymphocytic choriomeningitis virus (LCMV).

46. The composition of claim 44 or 45, wherein the one or more immunostimulatory molecules are directly or indirectly covalently linked to the MHC class II molecule.

47. The composition of any one of claims 44-46, wherein the one or more immunostimulatory molecules are directly or indirectly covalently linked to the MHC class II α chain or the portion thereof and/or the MHC class II β chain or the portion thereof.

48. The composition of any preceding claim, wherein the MHC class II molecule is a human MHC class II molecule.

49. The composition of claim 48, wherein the human MHC class II molecule is selected from the group consisting of HLA-DQ, HLA-DR, and HLA-DP.

50. The composition of claim 49, wherein the human MHC class II molecule is an HLA-DQ2 molecule.

51. The composition of claim 49, wherein the human MHC class II molecule is an HLA-DR2 molecule.

52. The composition of any preceding claim, wherein the MHC class II α chain or the portion thereof comprises an MHC class II α chain extracellular domain, and the MHC class II β chain or the portion thereof comprises an MHC class II β chain extracellular domain,
wherein the peptide linker linking the MHC ligand peptide to the MHC class II molecule is a flexible linker between about 9 and about 50 amino acids in length that comprises the first cysteine and is connected to the N-terminal end of the MHC class II β chain or the portion thereof,
wherein the second cysteine is in the MHC class II α chain or the portion thereof and is not present in a wild type MHC class II molecule corresponding to the MHC class II molecule in the composition, and
wherein the MHC class II molecule lacks a cysteine present in a corresponding wild type MHC class II molecule.

53. The composition of claim 52, wherein the composition is soluble,
wherein the MHC class II α chain or the portion thereof comprises the α1 domain and an α2 domain but does not comprise a transmembrane domain or a cytoplasmic domain,
wherein the MHC class II β chain or the portion thereof comprises the β1 domain and a β2 domain but does not comprise a transmembrane domain or a cytoplasmic domain, and
wherein the MHC class II α chain or the portion thereof and the MHC class II β chain or the portion thereof are linked by a Jun-Fos zipper comprising a Jun leucine zipper dimerization motif and a Fos leucine zipper dimerization motif.

54. The composition of claim 52 or 53, wherein the second cysteine is at a position corresponding to position 101 in the sequence set forth in SEQ ID NO: 49 when the MHC class II α chain or the portion thereof is optimally aligned with SEQ ID NO: 49, and
wherein the cysteine in the corresponding wild type MHC class II molecule is at a position corresponding to position 70 in the sequence set forth in SEQ ID NO: 49 when the MHC class II α chain or the portion thereof is optimally aligned with SEQ ID NO: 49.

55. The composition of any one of claims 52-54, wherein the MHC class II molecule is a human MHC class II molecule is selected from the group consisting of HLA-DQ, HLA-DP, and HLA-DR.

56. The composition of claim 55, wherein the human MHC class II molecule is HLA-DQ.

57. A nucleic acid encoding the composition of any preceding claim.

58. A method of eliciting an immune response in a subject, comprising administering to the subject an effective amount of the composition of any one of claims 1-56 or a nucleic acid encoding the composition.

59. A method of generating an antigen-binding protein that specifically binds an antigenic composition comprising an MHC ligand peptide covalently attached to an MHC class II molecule, comprising:
(a) immunizing a non-human animal with the composition of any one of claims 1-56 or a nucleic acid encoding the composition; and
(b) maintaining the non-human animal in conditions sufficient for the non-human animal to mount an immune response to the composition.

60. A method of generating an antigen-binding protein, comprising:
(a) immunizing a non-human animal with the composition of any one of claims 1-56 or a nucleic acid encoding the composition; and
(b) maintaining the non-human animal in conditions sufficient for the non-human animal to mount an immune response to the composition.

61. The method of claim 60, wherein the antigen-binding protein specifically binds an antigenic composition comprising an MHC ligand peptide covalently attached to an MHC class II molecule.


SEQUENCE LISTING (73 sequences)

SEQ ID NO: 1 - protein, 6 aa, Artificial Sequence
  Synthetic
SGGGGG

SEQ ID NO: 2 - protein, 5 aa, Artificial Sequence
  Synthetic
GSGGS

SEQ ID NO: 3 - protein, 4 aa, Artificial Sequence
  Synthetic
GGGS

SEQ ID NO: 4 - protein, 5 aa, Artificial Sequence
  Synthetic
GGGGS

SEQ ID NO: 5 - protein, 4 aa, Artificial Sequence
  Synthetic
GGSG

SEQ ID NO: 6 - protein, 5 aa, Artificial Sequence
  Synthetic
GGSGG

SEQ ID NO: 7 - protein, 5 aa, Artificial Sequence
  Synthetic
GSGSG

SEQ ID NO: 8 - protein, 5 aa, Artificial Sequence
  Synthetic
GSGGG

SEQ ID NO: 9 - protein, 5 aa, Artificial Sequence
  Synthetic
GGGSG

SEQ ID NO: 10 - protein, 5 aa, Artificial Sequence
  Synthetic
GSSSG

SEQ ID NO: 11 - protein, 6 aa, Artificial Sequence
  Synthetic
SGGGGG

SEQ ID NO: 12 - protein, 15 aa, Artificial Sequence
  Synthetic
GCGASGGGGSGGGGS

SEQ ID NO: 13 - protein, 15 aa, Artificial Sequence
  Synthetic
GCGASGGGGSGGGGS

SEQ ID NO: 14 - protein, 10 aa, Artificial Sequence
  Synthetic
GGGGSGGGGS

SEQ ID NO: 15 - protein, 15 aa, Artificial Sequence
  Synthetic
GGGASGGGGSGGGGS

SEQ ID NO: 16 - protein, 15 aa, Artificial Sequence
  Synthetic
GGGGSGGGGSGGGGS

SEQ ID NO: 17 - protein, 10 aa, Artificial Sequence
  Synthetic
GGGASGGGGS

SEQ ID NO: 18 - protein, 15 aa, Artificial Sequence
  Synthetic
GGGGSGGGGSGGGGS

SEQ ID NO: 19 - protein, 20 aa, Artificial Sequence
  Synthetic
GGGGSGGGGSGGGGSGGGGS

SEQ ID NO: 20 - protein, 5 aa, Artificial Sequence
  Synthetic
GCGGS

SEQ ID NO: 21 - protein, 15 aa, Artificial Sequence
  Synthetic
GCGGSGGGGSGGGGS

SEQ ID NO: 22 - protein, 6 aa, Artificial Sequence
  Synthetic
ENLYFQ

SEQ ID NO: 23 - protein, 39 aa, Artificial Sequence
  Synthetic
LTDTLQAETDQLEDEKSALQTEIANLLKEKEKLEFILAA

SEQ ID NO: 24 - protein, 40 aa, Artificial Sequence
  Synthetic
RIARLEEKVKTLKAQNSELASTANMLREQVAQLKQKVMNH

SEQ ID NO: 25 - protein, 13 aa, Artificial Sequence
  Synthetic
AKFVAAWTLKAAA

SEQ ID NO: 26 - protein, 15 aa, Artificial Sequence
  Synthetic
TMFEALPHIIDEVIN

SEQ ID NO: 27 - protein, 15 aa, Artificial Sequence
  Synthetic
GIKAVYNFATCGIFA

SEQ ID NO: 28 - protein, 15 aa, Artificial Sequence
  Synthetic
DIYKGVYQFKSVEFD

SEQ ID NO: 29 - protein, 15 aa, Artificial Sequence
  Synthetic
TSAFNKKTFDHTLMS

SEQ ID NO: 30 - protein, 15 aa, Artificial Sequence
  Synthetic
DAQSAQSQCRTFRGR

SEQ ID NO: 31 - protein, 15 aa, Artificial Sequence
  Synthetic
TFRGRVLDMFRTAFG

SEQ ID NO: 32 - protein, 15 aa, Artificial Sequence
  Synthetic
CDMLRLIDYNKAALS

SEQ ID NO: 33 - protein, 15 aa, Artificial Sequence
  Synthetic
IEQEADNMITEMLRK

SEQ ID NO: 34 - protein, 15 aa, Artificial Sequence
  Synthetic
EVKSFQWTQALRREL

SEQ ID NO: 35 - protein, 15 aa, Artificial Sequence
  Synthetic
KNVLKVGRLSAEELM

SEQ ID NO: 36 - protein, 15 aa, Artificial Sequence
  Synthetic
SERPQASGVYMGNLT

SEQ ID NO: 37 - protein, 15 aa, Artificial Sequence
  Synthetic
PSLTMACMAKQSQTP

SEQ ID NO: 38 - protein, 15 aa, Artificial Sequence
  Synthetic
EGWPYIACRTSIVGR

SEQ ID NO: 39 - protein, 15 aa, Artificial Sequence
  Synthetic
SQNRKDIKLIDVEMT

SEQ ID NO: 40 - protein, 15 aa, Artificial Sequence
  Synthetic
GWLCKMHTGIVRDKK

SEQ ID NO: 41 - protein, 15 aa, Artificial Sequence
  Synthetic
SCKSCWQKFDSLVRC

SEQ ID NO: 42 - protein, 15 aa, Artificial Sequence
  Synthetic
GLNDIFEAQKIEWHE

SEQ ID NO: 43 - protein, 26 aa, Artificial Sequence
  Synthetic
EQKLISEEDLEQKLISEEDLHHHHHH

SEQ ID NO: 44 - protein, 12 aa, Artificial Sequence
  Synthetic
QLQPFPQPELPY

SEQ ID NO: 45 - protein, 12 aa, Artificial Sequence
  Synthetic
FPQPEQPFPWQP

SEQ ID NO: 46 - protein, 12 aa, Artificial Sequence
  Synthetic
PQPELPYPQPQL

SEQ ID NO: 47 - protein, 16 aa, Artificial Sequence
  Synthetic
GGGGSENLYFQGGGGS

SEQ ID NO: 48 - protein, 28 aa, Artificial Sequence
  Synthetic
EQKLISEEDLGGEQKLISEEDLHHHHHH

SEQ ID NO: 49 - protein, 254 aa, Artificial Sequence
  Synthetic
MILNKALMLGALALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 50 - protein, 261 aa, Artificial Sequence
  Synthetic
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 51 - protein, 260 aa, Artificial Sequence
  Synthetic
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 52 - protein, 254 aa, Artificial Sequence
  Synthetic
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 53 - protein, 254 aa, Artificial Sequence [sequence omitted]

SEQ ID NO: 54 - protein, 254 aa, Artificial Sequence
  Synthetic
MILNKALMLGALALTTVMSPCGGEDIVADHVASYGVNLYQSYGPSGQYTHEFDGDEQFYVDLGRKETVWALPVLRQFRFDPQFALTNIAVLKHNLNSLIKCSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 55 - protein, 254 aa, Artificial Sequence
  Synthetic
MILNKALMLGALALTTVMSPCGGEDIVADHVASYGVNLYQSYGPSGQYTHEFDGDEQFYVDLGRKETVWQLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 56 - protein, 254 aa, Artificial Sequence
  Synthetic
MILNKALMLGALALTTVMSPCGGEDIVADHVASYGVNLYQSYGPSGQYTHEFDGDEQFYVDLGRKETVWALPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 57 - protein, 254 aa, Artificial Sequence
  Synthetic
MILNKALMLGALALTTVMSPCGGEDIVADHVASYGVNLYQSYGPSGQYTHEFDGDEQFYVDLGRKETVWQLPVLRQFRFDPQFALTNIAVLKHNLNSLIKCSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 58 - protein, 254 aa, Artificial Sequence
  Synthetic
MAISGVPVLGFFIIAVLMSAQESWAIKEEHVIIQAEFYLNPDQSGEFMFDFDGDEIFHVDMAKKETVWRLEEFGRFASCEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 59 - protein, 193 aa, Artificial Sequence
  Synthetic
EDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTE

SEQ ID NO: 60 - protein, 198 aa, Artificial Sequence
  Synthetic
RDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSK

SEQ ID NO: 61 - protein, 194 aa, Artificial Sequence
  Synthetic
AGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTE

SEQ ID NO: 62 - protein, 191 aa, Artificial Sequence
  Synthetic
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTE

SEQ ID NO: 63 - protein, 193 aa, Artificial Sequence
  Synthetic
EDIVADHVASYGVNLYQSYGPSGQYTHEFDGDEQFYVDLGRKETVWCLPVLRQFRFDPQFALTNIAVLKHNLNSLIKCSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTE

SEQ ID NO: 64 - protein, 193 aa, Artificial Sequence
  Synthetic
EDIVADHVASYGVNLYQSYGPSGQYTHEFDGDEQFYVDLGRKETVWALPVLRQFRFDPQFALTNIAVLKHNLNSLIKCSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTE

SEQ ID NO: 65 - protein, 193 aa, Artificial Sequence
  Synthetic
EDIVADHVASYGVNLYQSYGPSGQYTHEFDGDEQFYVDLGRKETVWQLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTE

SEQ ID NO: 66 - protein, 193 aa, Artificial Sequence
  Synthetic
EDIVADHVASYGVNLYQSYGPSGQYTHEFDGDEQFYVDLGRKETVWALPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTE

SEQ ID NO: 67 - protein, 193 aa, Artificial Sequence
  Synthetic
EDIVADHVASYGVNLYQSYGPSGQYTHEFDGDEQFYVDLGRKETVWQLPVLRQFRFDPQFALTNIAVLKHNLNSLIKCSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTE

SEQ ID NO: 68 - protein, 191 aa, Artificial Sequence
  Synthetic
IKEEHVIIQAEFYLNPDQSGEFMFDFDGDEIFHVDMAKKETVWRLEEFGRFASCEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTE

SEQ ID NO: 69 - protein, 12 aa, Artificial Sequence
  Synthetic
QPFPQPELPYPQ

SEQ ID NO: 70 - protein, 12 aa, Artificial Sequence
  Synthetic
QPFPQPEQPFPW

SEQ ID NO: 71 - protein, 10 aa, Artificial Sequence
  Synthetic
QPFPQPELPY

SEQ ID NO: 72 - protein, 10 aa, Artificial Sequence
  Synthetic
FPQPELPYPQ

SEQ ID NO: 73 - protein, 10 aa, Artificial Sequence
  Synthetic
FPQPEQPFPW